(19) United States
(12) Patent Application Publication
(10) Pub. No.: US 2024/0109961 A1
(43) Pub. Date: Apr. 4, 2024

(54) TREATMENT OF CHRONIC PRURIGO

(71) Applicant: Celldex Therapeutics, Inc., Hampton, NJ (US)

(72) Inventors: Bret Alan Holley, Pelham, NY (US); Joel Goldstein, Hopewell, NJ (US)

(73) Assignee: Celldex Therapeutics, Inc., Hampton, NJ (US)

(21) Appl. No.: 18/273,683

(22) PCT Filed: Jan. 21, 2022
(86) PCT No.: PCT/US2022/013380

§ 371 Date: Jul. 21, 2023

Related U.S. Application Data
(60) Provisional application No. 63/238,688, filed on Aug. 30, 2021.
(60) Provisional application No. 63/140,621, filed on Jan. 22, 2021.

(51) Int. Cl. C07K 16/28; A61P 17/04

(52) U.S. Cl. CPC C07K 16/2803; A61P 17/04; A61K 2039/505; C07K 2317/52

(57) ABSTRACT

Provided herein are methods and uses of inhibitors of KIT, a receptor tyrosine kinase, for example, antibodies and antigen binding fragments that immunospecifically bind to KIT, for managing, treating, or preventing chronic prurigo, including prurigo nodularis (PN). Also provided are improved antibodies for use in such methods and uses.

### CROSS-REFERENCE TO RELATED APPLICATIONS

This application claims the benefit of U.S. Provisional Patent Application Nos. 63/140,621, filed Jan. 22, 2021, and 63/238,688, filed Aug. 30, 2021, which are incorporated by reference herein in their entireties.

### REFERENCE TO SEQUENCE LISTING SUBMITTED ELECTRONICALLY

This application incorporates by reference a Sequence Listing submitted with this application as text file entitled “Seqlisting_12638-171-228.txt” created on Jan. 6, 2022 and having a size of 44,163 bytes.

### 1. FIELD

Provided herein are methods and uses of inhibitors of KIT, a receptor tyrosine kinase, for example, antibodies and antigen binding fragments that immunospecifically bind to KIT, for managing, treating, or preventing chronic prurigo, including prurigo nodularis (PN). Also provided are improved antibodies for use in such methods and uses.

### 2. BACKGROUND

KIT (or c-Kit) is a type III receptor tyrosine kinase encoded by the c-kit gene. KIT comprises five extracellular immunoglobulin (Ig)-like domains, a single transmembrane region, an inhibitory cytoplasmic juxtamembrane domain, and a split cytoplasmic kinase domain separated by a kinase insert segment (see, e.g., Yarden et al., Nature, 1986, 323:226-232; Ullrich and Schlessinger, Cell, 1990, 61:203-212; Clifford et al., J. Biol. Chem., 2003, 278:31461-31464). The human c-kit gene encoding the KIT receptor has been cloned as described by Yarden et al., EMBO J., 1987, 6:3341-3351. KIT is also known as CD117 or stem cell factor receptor (“SCFR”), because it is the receptor for the stem cell factor (“SCF”) ligand (also known as Steel Factor or Kit Ligand). SCF ligand binding to the first three extracellular Ig-like domains of KIT induces receptor dimerization, and thereby activates intrinsic tyrosine kinase activity through the phosphorylation of specific tyrosine residues in the juxtamembrane and kinase domains (see, e.g., Weiss and Schlessinger, Cell, 1998, 94:277-280; Clifford et al., J. Biol. Chem., 2003, 278:31461-31464). Members of the Stat, Src, ERK, and AKT signaling pathways have been shown to be downstream signal transducers of KIT signaling.

The fourth (D4) and fifth (D5) extracellular Ig-like domains of KIT are believed to mediate receptor dimerization (see, e.g., International Patent Application Publication No. WO 2008/153926; Yuzawa et al., Cell, 2007, 130:323-334).

Expression of KIT has been detected in various cell types, such as mast cells, stem cells, brain cells, melanoblasts, ovary cells, and cancer cells (e.g., leukemia cells) (see, e.g., Besmer, P. Curr. Opin. Cell Biol, 1991, 3:939-946; Lyman et al., Blood, 1998, 91: 1101-1134; Ashman, L. K., Int. J. Biochem. Cell Biol, 1999, 31: 1037-1051; Kitamura et al., Mutat. Res., 2001, 477: 165-171; Mol et al., J. Biol. Chem., 2003, 278:31461-31464). Moreover, KIT plays an important role in hematopoiesis, melanogenesis, and gametogenesis (see Ueda et al., Blood, 2002, 99:3342-3349).

Antibodies against human KIT are known, for example from International Patent Publication No WO2014018625A1, which is herein incorporated by reference in its entirety.

Chronic prurigo (CPG) is a disease characterised by the presence of both chronic pruritus (itching) and multiple localized or generalized pruriginous skin lesions.

Prurigo nodularis (PN; also known as nodular prurigo) is a distinct clinical disease defined by the presence of both chronic pruritus and multiple localized or generalized, elevated, firm, and nodular skin lesions. Although the underlying cause of PN is unknown, both neural and immunologic processes appear to play a role in its development (Elmariah et al., Practical approaches for diagnosis and management of prurigo nodularis: US expert panel consensus, Journal of the American Academy of Dermatology, 2020, 50190-9622(20):32189-32187). Reference is made also to Periera et al., Journal of the European Academy of Dermatology and Venereology (JEADV), 2018, 32:1059-1065 and Zeidler et al., Acta Derm Venereol, 2018, 98:173-179.

There is a need for therapies to manage or treat chronic prurigo including prurigo nodularis effectively and also to provide improved antibodies for use in such treatment.

### 3. SUMMARY

In one aspect, provided herein is a method of protecting against, treating, or managing chronic prurigo in a subject, comprising administering to a subject in need thereof a therapeutically effective amount of an antibody which immunospecifically binds to human KIT, or an antigen binding fragment thereof.

In another aspect, provided herein is use of an antibody which immunospecifically binds to human KIT, or an antigen binding fragment thereof, for the manufacture of a medicament for protecting against, treating, or managing chronic prurigo in a subject.

In another aspect, provided herein is an antibody which immunospecifically binds to human KIT, or an antigen binding fragment thereof, for use in a method of protecting against, treating, or managing chronic prurigo in a subject.

In specific embodiments, the human KIT comprises the amino acid sequence of SEQ ID NO: 1.

In specific embodiments, the chronic prurigo is prurigo nodularis.

In specific embodiments, the antibody is a bivalent monospecific antibody. In specific embodiments, the antibody is a bispecific antibody.

In specific embodiments, the antibody is a humanized antibody.

In specific embodiments, the antibody comprises a modified (e.g., mutated) Fc region or domain.

In specific embodiments, the antibody has reduced Fc receptor binding activity (particularly reduced FcγR binding activity).

In specific embodiments, the antibody does not induce significant degranulation of FcgRI-expressing human mast cells.

In specific embodiments, the antibody does not show significant Fc receptor-dependent KIT agonist activity.

In specific embodiments, the antibody specifically binds to a D4 or D5 region of human KIT.

In specific embodiments, the antibody comprises (A) (i) a light chain variable region (“VL”) comprising VL CDR1, VL CDR2, and VL CDR3 comprising the amino acid sequences of SEQ ID NO: 2, SEQ ID NO: 3, and SEQ ID NO: 4, respectively; and (ii) a heavy chain variable region (“VH”) comprising VH CDR1, VH CDR2, and VH CDR3 comprising the amino acid sequences of SEQ ID NO: 5, SEQ ID NO: 6, and SEQ ID NO: 7, respectively; (B) (i) a VL comprising VL CDR1, VL CDR2, and VL CDR3 comprising the amino acid sequences of SEQ ID NO: 2, SEQ ID NO: 3, and SEQ ID NO: 4, respectively; and (ii) a VH comprising VH CDR1, VH CDR2, and VH CDR3 comprising the amino acid sequences of SEQ ID NO: 25, SEQ ID NO: 26, and SEQ ID NO: 27, respectively; (C) (i) a VL comprising VL CDR1, VL CDR2, and VL CDR3 comprising the amino acid sequences of SEQ ID NO: 28, SEQ ID NO: 29, and SEQ ID NO: 30, respectively; and (ii) a VH comprising VH CDR1, VH CDR2, and VH CDR3 comprising the amino acid sequences of SEQ ID NO: 25, SEQ ID NO: 31, and SEQ ID NO: 32, respectively; (D) (i) a VL comprising VL CDR1, VL CDR2, and VL CDR3 comprising the amino acid sequences of SEQ ID NO: 2, SEQ ID NO: 3, and SEQ ID NO: 4, respectively; and (ii) a VH comprising VH CDR1, VH CDR2, and VH CDR3 comprising the amino acid sequences of SEQ ID NO: 33, SEQ ID NO: 34, and SEQ ID NO: 27, respectively; or (E) (i) a VL comprising VL CDR1, VL CDR2, and VL CDR3 comprising the amino acid sequences of SEQ ID NO: 35, SEQ ID NO: 36, and SEQ ID NO: 37, respectively; and (ii) a VH comprising VH CDR1, VH CDR2, and VH CDR3 comprising the amino acid sequences of SEQ ID NO: 38, SEQ ID NO: 39, and SEQ ID NO: 40, respectively.

In specific embodiments, the antibody comprises a light chain variable region (“VL”) comprising VL CDRs 1-3 comprising the amino acid sequences of SEQ ID NOs: 2-4, respectively, and a heavy chain variable region (“VH”) comprising VH CDRs 1-3 comprising the amino acid sequences of SEQ ID NOs: 5-7, respectively.

In specific embodiments, the antibody comprises (i) a VL comprising the amino acid sequence: DIVMTQSPSX_{K1}SASVGDRVTITCKASQNVRTNVAWYQQKPGKAPKX_{K2}LIYSASYRYS GVPDRFX_{K3}GSGSGTDFTLTISSLQX_{K4}EDFAX_{K5}YX_{K6}CQQYNSYPRTFGGGTKVEIK (SEQ ID NO: 17), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and (ii) a VH comprising the amino acid sequence: QVQLVQSGAEX_{H1}KKPGASVKX_{H2}SCKASGYTFTDYYINWVX_{H3}QAPGKGLEWIARIYPG SGNTYYNEKFKGRX_{H4}TX_{H5}TAX_{H6}K STSTAYMX_{H7}LSSLRSEDX_{H8}AVYFCARGVYYFDY WGQGTTVTVSS (SEQ ID NO: 18), wherein Xxi is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain. In specific embodiments, X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

In specific embodiments, the antibody comprises a VL comprising an amino acid sequence selected from the group consisting of SEQ ID NOs: 13, 14, 15, and 16; and a VH comprising an amino acid sequence selected from the group consisting of SEQ ID NOs: 8, 9, 10, 11, and 12.

In specific embodiments, the antibody comprises a human light chain constant region. In specific embodiments, the antibody comprises a human heavy chain constant region. In specific embodiments, the human heavy chain constant region is a human IgG constant region. In specific embodiments, the human heavy chain constant region is a human IgG1 constant region.

In specific embodiments, the antibody comprises a modified (e.g., mutated) human Fc region or domain. In specific embodiments, the antibody comprises a modified (e.g., mutated) human IgG1 Fc region or domain. In specific embodiments, the modified (e.g., mutated) human IgG1 Fc region or domain comprises non-naturally occurring amino acids 234A, 235Q and 322Q as numbered by the EU index as set forth in Kabat. In specific embodiments, the modified (e.g., mutated) human IgG1 Fc region or domain further comprises non-naturally occurring amino acids 252Y, 254T and 256E as numbered by the EU index as set forth in Kabat.

In specific embodiments, the antibody comprises: (i) a VL comprising an amino acid sequence of SEQ ID NO: 14; (ii) a VH comprising an amino acid sequence of SEQ ID NO: 10; and (iii) a modified (e.g., mutated) human IgG1 Fc region or domain comprising non-naturally occurring amino acids 234A, 235Q and 322Q as numbered by the EU index as set forth in Kabat.

In specific embodiments, the antibody comprises: (i) a VL comprising an amino acid sequence of SEQ ID NO: 14; (ii) a VH comprising an amino acid sequence of SEQ ID NO: 10; and (iii) a modified (e.g., mutated) human IgG1 Fc region or domain comprising non-naturally occurring amino acids 234A, 235Q, 322Q, 252Y, 254T and 256E as numbered by the EU index as set forth in Kabat.

In specific embodiments, the antibody comprises a heavy chain comprising the amino acid sequence:

(SEQ ID NO: 21)
QVQLVQSGAEVKKPGASVKLSCKASGYTFTDYYINWVRQAPGKGLEWIAR
IYPGSGNTYYNEKFKGRATLTADKSTSTAYMQLSSLRSEDTAVYFCARGV
YYFDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAQGGPSVFLFPPKPKDTL
YITREPEVTCVVVDVSHEDPEVKFNWYVDGVEVHNAKTKPREEQYNSTYR
VVSVLTVLHQDWLNGKEYKCQVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.

In specific embodiments, the antibody comprises a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In specific embodiments, the antibody comprises a heavy chain comprising the amino acid sequence: QVQLVQSGAEVKKPGASVKLSCKASGYTFTDYYINWVRQAPGKGLEWIARIYPGSGNT YYNEKFKGRATLTADKSTSTAYMQLSSLRSEDTAVYFCARGVYYFDYWGQGTTVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAQG GPSVFLFPPKPKDTLYITREPEVTCVVVDVSHEDPEVKFNWYVDGVEVHNAKTKPREEQ YNSTYRVVSVLTVLHQDWLNGKEYKCQVSNKALPAPIEKTISKAKGQPREPQVYTLPPS RDELTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 21); and a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In specific embodiments, the subject is a human adult. In specific embodiments, the subject is a human child.

### 3.1 Illustrative Embodiments

### 3.1.1 Set 1

1. A method of protecting against, treating, or managing chronic prurigo in a subject, comprising administering to a subject in need thereof a therapeutically effective amount of an antibody which immunospecifically binds to human KIT, or an antigen binding fragment thereof.
2. The method of embodiment 1, wherein the human KIT comprises the amino acid sequence of SEQ ID NO: 1.
3. The method of embodiment 1 or 2, wherein the chronic prurigo is prurigo nodularis.
4. The method of any one of embodiments 1 to 3, wherein the antibody is a bivalent monospecific antibody.
5. The method of any one of embodiments 1 to 3, wherein the antibody is a bispecific antibody.
6. The method of any one of embodiments 1 to 5, wherein the antibody is a humanized antibody.
7. The method of any one of embodiments 1 to 6, wherein the antibody comprises a modified (e.g., mutated) Fc region or domain.
8. The method of any one of embodiments 1 to 7, wherein the antibody has reduced Fc receptor binding activity.
9. The method of any one of embodiments 1 to 8, wherein the antibody has reduced Fc yR binding activity.
10. The method of any one of embodiments 1 to 9, wherein the antibody does not induce significant degranulation of FcgRI-expressing human mast cells.
11. The method of any one of embodiments 1 to 10, wherein the antibody does not show significant Fc receptor-dependent KIT agonist activity.
12. The method of any one of embodiments 1 to 11, wherein the antibody specifically binds to a D4 or D5 region of human KIT.
13. The method of any one of embodiments 1 to 12, wherein the antibody comprises:
(A) (i) a light chain variable region (“VL”) comprising VL CDR1, VL CDR2, and VL CDR3 comprising the amino acid sequences of SEQ ID NO: 2, SEQ ID NO: 3, and SEQ ID NO: 4, respectively; and
   (ii) a heavy chain variable region (“VH”) comprising VH CDR1, VH CDR2, and VH CDR3 comprising the amino acid sequences of SEQ ID NO: 5, SEQ ID NO: 6, and SEQ ID NO: 7, respectively;
(B) (i) a VL comprising VL CDR1, VL CDR2, and VL CDR3 comprising the amino acid sequences of SEQ ID NO: 2, SEQ ID NO: 3, and SEQ ID NO: 4, respectively; and
   (ii) a VH comprising VH CDR1, VH CDR2, and VH CDR3 comprising the amino acid sequences of SEQ ID NO: 25, SEQ ID NO: 26, and SEQ ID NO: 27, respectively;
(C) (i) a VL comprising VL CDR1, VL CDR2, and VL CDR3 comprising the amino acid sequences of SEQ ID NO: 28, SEQ ID NO: 29, and SEQ ID NO: 30, respectively; and
   (ii) a VH comprising VH CDR1, VH CDR2, and VH CDR3 comprising the amino acid sequences of SEQ ID NO: 25, SEQ ID NO: 31, and SEQ ID NO: 32, respectively;
(D) (i) a VL comprising VL CDR1, VL CDR2, and VL CDR3 comprising the amino acid sequences of SEQ ID NO: 2, SEQ ID NO: 3, and SEQ ID NO: 4, respectively; and
   (ii) a VH comprising VH CDR1, VH CDR2, and VH CDR3 comprising the amino acid sequences of SEQ ID NO: 33, SEQ ID NO: 34, and SEQ ID NO: 27, respectively; or
(E) (i) a VL comprising VL CDR1, VL CDR2, and VL CDR3 comprising the amino acid sequences of SEQ ID NO: 35, SEQ ID NO: 36, and SEQ ID NO: 37, respectively; and
   (ii) a VH comprising VH CDR1, VH CDR2, and VH CDR3 comprising the amino acid sequences of SEQ ID NO: 38, SEQ ID NO: 39, and SEQ ID NO: 40, respectively.
      14. The method of embodiment 13, wherein the antibody comprises a VL comprising VL CDRs 1-3 comprising the amino acid sequences of SEQ ID NOs: 2-4, respectively, and a VH comprising VH CDRs 1-3 comprising the amino acid sequences of SEQ ID NOs: 5-7, respectively.
      15. The method of any one of embodiments 1 to 14, wherein the antibody comprises
(i) a VL comprising the amino acid sequence:
DIVMTQSPSX_{K1}LSASVGDRVTITCKASQNVRTNVAWYQQKPGKAPKX_{K2}LIYSASYRYS GVPDRFX_{K3}GSGSGTDFTLTISSLQX_{K4}EDFAX_{K5}YX_{X6}CQQYNSYPRTFGGGTKVEIK (SEQ ID NO: 17), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and
(ii) a VH comprising the amino acid sequence:
QVQLVQSGAEX_{H1}KKPGASVKX_{H2}SCKASGYTFTDYYINWVX_{H3}QAPGKGLEWIARIYPG SGNTYYNEKFKGRX_{H4}TX_{H5}TAX_{H6}KSTSTAYMX_{H7}LSSLRSEDX_{H8}AVYFCARGVYYFDY WGQGTTVTVSS (SEQ ID NO: 18), wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain.
   16. The method of embodiment 15, wherein X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.
   17. The method of any one of embodiments 1 to 16, wherein the antibody comprises a VL comprising an amino acid sequence selected from the group consisting of SEQ ID NOs: 13, 14, 15, and 16; and a VH comprising an amino acid sequence selected from the group consisting of SEQ ID NOs: 8, 9, 10, 11, and 12.
   18. The method of any one of embodiments 1 to 17, wherein the antibody comprises a human light chain constant region.
   19. The method of any one of embodiments 1 to 18, wherein the antibody comprises a human heavy chain constant region.
   20. The method of embodiment 19, wherein the human heavy chain constant region is a human IgG constant region.
   21. The method of embodiment 20, wherein the human heavy chain constant region is a human IgG1 constant region.
   22. The method of any one of embodiments 1 to 21, wherein the antibody comprises a modified (e.g., mutated) human Fc region or domain.
   23. The method of any one of embodiments 1 to 21, wherein the antibody comprises a modified (e.g., mutated) human IgG1 Fc region or domain.
   24. The method of embodiment 23, wherein the modified (e.g., mutated) human IgG1 Fc region or domain comprises non-naturally occurring amino acids 234A, 235Q and 322Q as numbered by the EU index as set forth in Kabat.
   25. The method of embodiment 24, wherein the modified (e.g., mutated) human IgG1 Fc region or domain further comprises non-naturally occurring amino acids 252Y, 254T and 256E as numbered by the EU index as set forth in Kabat.
   26. The method of any one of embodiments 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q and 322Q as numbered by the EU index as set forth in Kabat.
   27. The method of any one of embodiments 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q, 322Q, 252Y, 254T and 256E as numbered by the EU index as set forth in Kabat.
   28. The method of any one of embodiments 1 to 25, wherein the antibody comprises a heavy chain comprising the amino acid sequence:

(SEQ ID NO: 21)
QVQLVQSGAEVKKPGASVKLSCKASGYTFTDYYINWVRQAPGKGLEWIAR
IYPGSGNTYYNEKFKGRATLTADKSTSTAYMQLSSLRSEDTAVYFCARGV
YYFDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAQGGPSVFLFPPKPKDTL
YITREPEVTCVVVDVSHEDPEVKFNWYVDGVEVHNAKTKPREEQYNSTYR
VVSVLTVLHQDWLNGKEYKCQVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.

29. The method of any one of embodiments 1 to 25, wherein the antibody comprises a light chain comprising the amino acid sequence:

(SEQ ID NO: 22)
DIVMTQSPSSLSASVGDRVTITCKASQNVRINVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

30. The method of any one of embodiments 1 to 25, wherein the antibody comprises a heavy chain comprising the amino acid sequence:
QVQLVQSGAEVKKPGASVKLSCKASGYTFTDYYINWVRQAPGKGLEWIARIYPGSGNT YYNEKFKGRATLTADKSTSTAYMQLSSLRSEDTAVYFCARGVYYFDYWGQGTTVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAQG GPSVFLFPPKPKDTLYITREPEVTCVVVDVSHEDPEVKFNWYVDGVEVHNAKTKPREEQ YNSTYRVVSVLTVLHQDWLNGKEYKCQVSNKALPAPIEKTISKAKGQPREPQVYTLPPS RDELTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 21); and a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

31. The method of any one of embodiments 1 to 30, wherein the subject is a human adult.
32. The method of any one of embodiments 1 to 30, wherein the subject is a human child.

### 3.1.2 Set 2

1. Use of an antibody which immunospecifically binds to human KIT, or an antigen binding fragment thereof, for the manufacture of a medicament for protecting against, treating, or managing chronic prurigo in a subject.
2. The use of embodiment 1, wherein the human KIT comprises the amino acid sequence of SEQ ID NO: 1.
3. The use of embodiment 1 or 2, wherein the chronic prurigo is prurigo nodularis.
4. The use of any one of embodiments 1 to 3, wherein the antibody is a bivalent monospecific antibody.
5. The use of any one of embodiments 1 to 3, wherein the antibody is a bispecific antibody.
6. The use of any one of embodiments 1 to 5, wherein the antibody is a humanized antibody.
7. The use of any one of embodiments 1 to 6, wherein the antibody comprises a modified (e.g., mutated) Fc region or domain.
8. The use of any one of embodiments 1 to 7, wherein the antibody has reduced Fc receptor binding activity.
9. The use of any one of embodiments 1 to 8, wherein the antibody has reduced FcγR binding activity.
10. The use of any one of embodiments 1 to 9, wherein the antibody does not induce significant degranulation of FcgRI-expressing human mast cells.
11. The use of any one of embodiments 1 to 10, wherein the antibody does not show significant Fc receptor-dependent KIT agonist activity.
12. The use of any one of embodiments 1 to 11, wherein the antibody specifically binds to a D4 or D5 region of human KIT.
13. The use of any one of embodiments 1 to 12, wherein the antibody comprises:
(A) (i) a light chain variable region (“VL”) comprising VL CDR1, VL CDR2, and VL CDR3 comprising the amino acid sequences of SEQ ID NO: 2, SEQ ID NO: 3, and SEQ ID NO: 4, respectively; and
   (ii) a heavy chain variable region (“VH”) comprising VH CDR1, VH CDR2, and VH CDR3 comprising the amino acid sequences of SEQ ID NO: 5, SEQ ID NO: 6, and SEQ ID NO: 7, respectively;
(B) (i) a VL comprising VL CDR1, VL CDR2, and VL CDR3 comprising the amino acid sequences of SEQ ID NO: 2, SEQ ID NO: 3, and SEQ ID NO: 4, respectively; and
   (ii) a VH comprising VH CDR1, VH CDR2, and VH CDR3 comprising the amino acid sequences of SEQ ID NO: 25, SEQ ID NO: 26, and SEQ ID NO: 27, respectively;
(C) (i) a VL comprising VL CDR1, VL CDR2, and VL CDR3 comprising the amino acid sequences of SEQ ID NO: 28, SEQ ID NO: 29, and SEQ ID NO: 30, respectively; and
   (ii) a VH comprising VH CDR1, VH CDR2, and VH CDR3 comprising the amino acid sequences of SEQ ID NO: 25, SEQ ID NO: 31, and SEQ ID NO: 32, respectively;
(D) (i) a VL comprising VL CDR1, VL CDR2, and VL CDR3 comprising the amino acid sequences of SEQ ID NO: 2, SEQ ID NO: 3, and SEQ ID NO: 4, respectively; and
   (ii) a VH comprising VH CDR1, VH CDR2, and VH CDR3 comprising the amino acid sequences of SEQ ID NO: 33, SEQ ID NO: 34, and SEQ ID NO: 27, respectively; or
(E) (i) a VL comprising VL CDR1, VL CDR2, and VL CDR3 comprising the amino acid sequences of SEQ ID NO: 35, SEQ ID NO: 36, and SEQ ID NO: 37, respectively; and
   (ii) a VH comprising VH CDR1, VH CDR2, and VH CDR3 comprising the amino acid sequences of SEQ ID NO: 38, SEQ ID NO: 39, and SEQ ID NO: 40, respectively.
      14. The use of any one of embodiments 1 to 13 wherein the antibody comprises a VL comprising VL CDRs 1-3 comprising the amino acid sequences of SEQ ID NOs: 2-4, respectively, and a VH comprising VH CDRs 1-3 comprising the amino acid sequences of SEQ ID NOs: 5-7, respectively.
      15. The use of any one of embodiments 1 to 13, wherein the antibody comprises
(i) a VL comprising the amino acid sequence:
DIVMTQSPSX_{K1}LSASVGDRVTITCKASQNVRTNVAWYQQKPGKAPKX_{K2}LIYSASYRYS GVPDRFX_{K3}GSGSGTDFTLTISSLQX_{K4}EDFAX_{K5}YX_{K6}CQQYNSYPRTFGGGTKVEIK (SEQ ID NO: 17), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and
(ii) a VH comprising the amino acid sequence:
QVQLVQSGAEX_{H1}KKPGASVKX_{H2}SCKASGYTFTDYYINWVX_{H3}QAPGKGLEWIARIYPG SGNTYYNEKFKGRX_{H4}TX_{H5}TAX_{H6}KSTSTAYMX_{H7}LSSLRSEDX_{H8}AVYFCARGVYYFDY WGQGTTVTVSS (SEQ ID NO: 18), wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain.
   16. The use of embodiment 15, wherein X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.
   17. The use of any one of embodiments 1 to 16, wherein the antibody comprises a VL comprising an amino acid sequence selected from the group consisting of SEQ ID NOs: 13, 14, 15, and 16; and a VH comprising an amino acid sequence selected from the group consisting of SEQ ID NOs: 8, 9, 10, 11, and 12.
   18. The use of any one of embodiments 1 to 17, wherein the antibody comprises a human light chain constant region.
   19. The use of any one of embodiments 1 to 18, wherein the antibody comprises a human heavy chain constant region.
   20. The use of embodiment 19, wherein the human heavy chain constant region is a human IgG constant region.
   21. The use of embodiment 20, wherein the human heavy chain constant region is a human IgG1 constant region.
   22. The use of any one of embodiments 1 to 21, wherein the antibody comprises a modified (e.g., mutated) human Fc region or domain.
   23. The use of any one of embodiments 1 to 21, wherein the antibody comprises a modified (e.g., mutated) human IgG1 Fc region or domain.
   24. The use of embodiment 23, wherein the modified (e.g., mutated) human IgG1 Fc region or domain comprises non-naturally occurring amino acids 234A, 235Q and 322Q as numbered by the EU index as set forth in Kabat.
   25. The use of embodiment 24, wherein the modified (e.g., mutated) human IgG1 Fc region or domain further comprises non-naturally occurring amino acids 252Y, 254T and 256E as numbered by the EU index as set forth in Kabat.
   26. The use of any one of embodiments 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q and 322Q as numbered by the EU index as set forth in Kabat.
   27. The use of any one of embodiments 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q, 322Q, 252Y, 254T and 256E as numbered by the EU index as set forth in Kabat.
   28. The use of any one of embodiments 1 to 25, wherein the antibody comprises a heavy chain comprising the amino acid sequence:

(SEQ ID NO: 21)
QVQLVQSGAEVKKPGASVKLSCKASGYTFTDYYINWVRQAPGKGLEWIAR
IYPGSGNTYYNEKFKGRATLTADKSTSTAYMQLSSLRSEDTAVYFCARGV
YYFDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAQGGPSVFLFPPKPKDTL
YITREPEVTCVVVDVSHEDPEVKFNWYVDGVEVHNAKTKPREEQYNSTYR
VVSVLTVLHQDWLNGKEYKCQVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.

29. The use of any one of embodiments 1 to 25, wherein the antibody comprises a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

30. The use of any one of embodiments 1 to 25, wherein the antibody comprises a heavy chain comprising the amino acid sequence:
QVQLVQSGAEVKKPGASVKLSCKASGYTFTDYYINWVRQAPGKGLEWIARIYPGSGNT YYNEKFKGRATLTADKSTSTAYMQLSSLRSEDTAVYFCARGVYYFDYWGQGTTVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAQG GPSVFLFPPKPKDTLYITREPEVTCVVVDVSHEDPEVKFNWYVDGVEVHNAKTKPREEQ YNSTYRVVSVLTVLHQDWLNGKEYKCQVSNKALPAPIEKTISKAKGQPREPQVYTLPPS RDELTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 21); and a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

31. The use of any one of embodiments 1 to 30, wherein the subject is a human adult.
32. The use of any one of embodiments 1 to 30, wherein the subject is a human child.

### 3.1.3 Set 3

1. An antibody which immunospecifically binds to human KIT, or an antigen binding fragment thereof, for use in a method of protecting against, treating, or managing chronic prurigo in a subject.
2. The antibody or antigen binding fragment thereof for use of embodiment 1, wherein the human KIT comprises the amino acid sequence of SEQ ID NO: 1.
3. The antibody or antigen binding fragment thereof for use of embodiment 1 or 2, wherein the chronic prurigo is prurigo nodularis.
4. The antibody or antigen binding fragment thereof for use of any one of embodiments 1 to 3, wherein the antibody is a bivalent monospecific antibody.
5. The antibody or antigen binding fragment thereof for use of any one of embodiments 1 to 3, wherein the antibody is a bispecific antibody.
6. The antibody or antigen binding fragment thereof for use of any one of embodiments 1 to 5, wherein the antibody is a humanized antibody.
7. The antibody or antigen binding fragment thereof for use of any one of embodiments 1 to 6, wherein the antibody comprises a modified (e.g., mutated) Fc region or domain.
8. The antibody or antigen binding fragment thereof for use of any one of embodiments 1 to 7, wherein the antibody has reduced Fc receptor binding activity.
9. The antibody or antigen binding fragment thereof for use of any one of embodiments 1 to 8, wherein the antibody has reduced FcγR binding activity.
10. The antibody or antigen binding fragment thereof for use of any one of embodiments 1 to 9, wherein the antibody does not induce significant degranulation of FcgRI-expressing human mast cells.
11. The antibody or antigen binding fragment thereof for use of any one of embodiments 1 to 10, wherein the antibody does not show significant Fc receptor-dependent KIT agonist activity.
12. The antibody or antigen binding fragment thereof for use of any one of embodiments 1 to 11, wherein the antibody specifically binds to a D4 or D5 region of human KIT.
13. The antibody or antigen binding fragment thereof for use of any one of embodiments 1 to 12, wherein the antibody comprises:
(A) (i) a light chain variable region (“VL”) comprising VL CDR1, VL CDR2, and VL CDR3 comprising the amino acid sequences of SEQ ID NO: 2, SEQ ID NO: 3, and SEQ ID NO: 4, respectively; and
   (ii) a heavy chain variable region (“VH”) comprising VH CDR1, VH CDR2, and VH CDR3 comprising the amino acid sequences of SEQ ID NO: 5, SEQ ID NO: 6, and SEQ ID NO: 7, respectively;
(B) (i) a VL comprising VL CDR1, VL CDR2, and VL CDR3 comprising the amino acid sequences of SEQ ID NO: 2, SEQ ID NO: 3, and SEQ ID NO: 4, respectively; and
   (ii) a VH comprising VH CDR1, VH CDR2, and VH CDR3 comprising the amino acid sequences of SEQ ID NO: 25, SEQ ID NO: 26, and SEQ ID NO: 27, respectively;
(C) (i) a VL comprising VL CDR1, VL CDR2, and VL CDR3 comprising the amino acid sequences of SEQ ID NO: 28, SEQ ID NO: 29, and SEQ ID NO: 30, respectively; and
   (ii) a VH comprising VH CDR1, VH CDR2, and VH CDR3 comprising the amino acid sequences of SEQ ID NO: 25, SEQ ID NO: 31, and SEQ ID NO: 32, respectively;
(D) (i) a VL comprising VL CDR1, VL CDR2, and VL CDR3 comprising the amino acid sequences of SEQ ID NO: 2, SEQ ID NO: 3, and SEQ ID NO: 4, respectively; and
   (ii) a VH comprising VH CDR1, VH CDR2, and VH CDR3 comprising the amino acid sequences of SEQ ID NO: 33, SEQ ID NO: 34, and SEQ ID NO: 27, respectively; or
(E) (i) a VL comprising VL CDR1, VL CDR2, and VL CDR3 comprising the amino acid sequences of SEQ ID NO: 35, SEQ ID NO: 36, and SEQ ID NO: 37, respectively; and
   (ii) a VH comprising VH CDR1, VH CDR2, and VH CDR3 comprising the amino acid sequences of SEQ ID NO: 38, SEQ ID NO: 39, and SEQ ID NO: 40, respectively.
      14. The antibody or antigen binding fragment thereof for use of any one of embodiments 1 to 13, wherein the antibody comprises a VL comprising VL CDRs 1-3 comprising the amino acid sequences of SEQ ID NOs: 2-4, respectively, and a VH comprising VH CDRs 1-3 comprising the amino acid sequences of SEQ ID NOs: 5-7, respectively.
      15. The antibody or antigen binding fragment thereof for use of any one of embodiments 1 to 14, wherein the antibody comprises
(i) a VL comprising the amino acid sequence:
DIVMTQSPSX_{K1}LSASVGDRVTITCKASQNVRTNVAWYQQKPGKAPKX_{K2}LIYSASYRYS GVPDRFX_{K3}GSGSGTDFTLTISSLQX_{K4}EDFAX_{K5}YX_{K6}CQQYNSYPRTFGGGTKVEIK (SEQ ID NO: 17), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and
(ii) a VH comprising the amino acid sequence:
QVQLVQSGAEX_{H1}KKPGASVKX_{H2}SCKASGYTFTDYYINWVX_{H3}QAPGKGLEWIARIYPG SGNTYYNEKFKGRX_{H4}TX_{H5}TAX_{H6}KSTSTAYMX_{H7}LSSLRSEDX_{H8}AVYFCARGVYYFDY WGQGTTVTVSS (SEQ ID NO: 18), wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain.
   16. The antibody or antigen binding fragment thereof for use of embodiment 15, wherein X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.
   17. The antibody or antigen binding fragment thereof for use of any one of embodiments 1 to 16, wherein the antibody comprises a VL comprising an amino acid sequence selected from the group consisting of SEQ ID NOs: 13, 14, 15, and 16; and a VH comprising an amino acid sequence selected from the group consisting of SEQ ID NOs: 8, 9, 10, 11, and 12.
   18. The antibody or antigen binding fragment thereof for use of any one of embodiments 1 to 17, wherein the antibody comprises a human light chain constant region.
   19. The antibody or antigen binding fragment thereof for use of any one of embodiments 1 to 18, wherein the antibody comprises a human heavy chain constant region.
   20. The antibody or antigen binding fragment thereof for use of embodiment 19, wherein the human heavy chain constant region is a human IgG constant region.
   21. The antibody or antigen binding fragment thereof for use of embodiment 20, wherein the human heavy chain constant region is a human IgG1 constant region.
   22. The antibody or antigen binding fragment thereof for use of any one of embodiments 1 to 21, wherein the antibody comprises a modified (e.g., mutated) human Fc region or domain.
   23. The antibody or antigen binding fragment thereof for use of any one of embodiments 1 to 21, wherein the antibody comprises a modified (e.g., mutated) human IgG1 Fc region or domain.
   24. The antibody or antigen binding fragment thereof for use of embodiment 23, wherein the modified (e.g., mutated) human IgG1 Fc region or domain comprises non-naturally occurring amino acids 234A, 235Q and 322Q as numbered by the EU index as set forth in Kabat.
   25. The antibody or antigen binding fragment thereof for use of embodiment 24, wherein the modified (e.g., mutated) human IgG1 Fc region or domain further comprises non-naturally occurring amino acids 252Y, 254T and 256E as numbered by the EU index as set forth in Kabat.
   26. The antibody or antigen binding fragment thereof for use of any one of embodiments 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q and 322Q as numbered by the EU index as set forth in Kabat.
   27. The antibody or antigen binding fragment thereof for use of any one of embodiments 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q, 322Q, 252Y, 254T and 256E as numbered by the EU index as set forth in Kabat.
   28. The antibody or antigen binding fragment thereof for use of any one of embodiments 1 to 25, wherein the antibody comprises a heavy chain comprising the amino acid sequence:

(SEQ ID NO: 21)
QVQLVQSGAEVKKPGASVKLSCKASGYTFTDYYINWVRQAPGKGLEWIAR
IYPGSGNTYYNEKFKGRATLTADKSTSTAYMQLSSLRSEDTAVYFCARGV
YYFDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAQGGPSVFLFPPKPKDTL
YITREPEVTCVVVDVSHEDPEVKFNWYVDGVEVHNAKTKPREEQYNSTYR
VVSVLTVLHQDWLNGKEYKCQVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG.

29. The antibody or antigen binding fragment thereof for use of any one of embodiments 1 to 25, wherein the antibody comprises a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC.

30. The antibody or antigen binding fragment thereof for use of any one of embodiments 1 to 25, wherein the antibody comprises a heavy chain comprising the amino acid sequence:
QVQLVQSGAEVKKPGASVKLSCKASGYTFTDYYINWVRQAPGKGLEWIARIYPGSGNT YYNEKFKGRATLTADKSTSTAYMQLSSLRSEDTAVYFCARGVYYFDYWGQGTTVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAQG GPSVFLFPPKPKDTLYITREPEVTCVVVDVSHEDPEVKFNWYVDGVEVHNAKTKPREEQ YNSTYRVVSVLTVLHQDWLNGKEYKCQVSNKALPAPIEKTISKAKGQPREPQVYTLPPS RDELTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 21); and a light chain comprising the amino acid sequence:

(SEQ ID NO: 22)
DIVMTQSPSSLSASVGDRVTITCKASQNVRTNVAWYQQKPGKAPKALIYS
ASYRYSGVPDRFTGSGSGTDFTLTISSLQPEDFADYFCQQYNSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

31. The antibody or antigen binding fragment thereof for use of any one of embodiments 1 to 30, wherein the subject is a human adult.
32. The antibody or antigen binding fragment thereof for use of any one of embodiments 1 to 30, wherein the subject is a human child.

### 4. BRIEF DESCRIPTION OF THE FIGURES

FIG. 1 depicts the amino acid sequence of full length human KIT (SEQ ID NO: 1), GenBank™ accession number AAC50969. The first through fifth extracellular Ig-like domains (i.e., D1, D2, D3, D4, and D5) are indicated; “{” depicts the amino-terminal residue of each domain and “)” depicts the carboxyl-terminal residue of each domain. The D1 domain is depicted at P34 to R112, the D2 domain is depicted at D113 to P206, the D3 domain is depicted at A207 to D309, the D4 domain is depicted at K310 to N410, the hinge region between D4 and D5 is located at V409 to N410, and the D5 domain is depicted at T411 to K509. Also, the D1/D2 hinge region is located at D113 to L117; the D2/D3 hinge region is located at P206 to A210; and the D3/D4 hinge region is located at D309 to G311. The D4/D5 region comprises K310 to K509. The transmembrane domain comprises residues F525 to Q545, and the kinase domain comprises residues K589 to S933.

FIGS. 2A-2E depict effects of a particular anti-KIT antibody according to the present invention, mAb1, on plasma tryptase levels.

FIGS. 3A and 3B depict further effects of a particular anti-KIT antibody according to the present invention, mAb1, on plasma tryptase levels.

FIG. 4 depicts effects of a particular anti-KIT antibody according to the present invention, mAb1, on plasma Stem Cell Factor (SCF) levels.

FIG. 5 depicts effects of a particular anti-KIT antibody according to the present invention, mAb1, and a corresponding antibody with the same variable region sequences but an unmutated (wild type) human IgG1 sequence, mAbc, on SCF-induced activation of wild-type KIT and downstream intracellular signaling pathways.

FIG. 6 depicts effects of a particular anti-KIT antibody according to the present invention, mAb1, and a corresponding antibody with the same variable region sequences but an unmutated (wild type) human IgG1 sequence, mAbc, on SCF-dependent cell proliferation.

FIG. 7 shows the binding affinities of a particular anti-KIT antibody according to the present invention, mAb1, and a corresponding antibody with the same variable region sequences but an unmutated (wild type) human IgG1 sequence, mAbc, for recombinant human Fc-gamma receptors (FcγRs) and human neonatal Fc Receptor (FcRn).

FIGS. 8A-8N depict the binding curves of a particular anti-KIT antibody according to the present invention, mAb1, and a corresponding antibody with the same variable region sequences but an unmutated (wild type) human IgG1 sequence, mAbc, for recombinant human Fc-gamma receptors (FcγRs) and human neonatal Fc Receptor (FcRn). FIG. 8A depicts the binding curves of mAb1 for FcγRI. FIG. 8B depicts the binding curves of mAb1 for FcγRIIa. FIG. 8C depicts the binding curves of mAb1 for FcγRIIb. FIG. 8D depicts the binding curves of mAb1 for FcγRIIIa. FIG. 8E depicts the binding curves of mAb1 for FcγRIIIb. FIG. 8F depicts the binding curves of mAb1 for FcRn (pH 6.0). FIG. 8G depicts the binding curves of mAb1 for FcRn (pH 7.2). FIG. 8H depicts the binding curves of mAbc for FcγRI. FIG. 8I depicts the binding curves of mAbc for FcγRIIa. FIG. 8J depicts the binding curves of mAbc for FcγRIIb. FIG. 8K depicts the binding curves of mAbc for FcγRIIIa. FIG. 8L depicts the binding curves of mAbc for FcγRIIIb. FIG. 8M depicts the binding curves of mAbc for FcRn (pH 6.0). FIG. 8N depicts the binding curves of mAbc for FcRn (pH 7.2).

FIG. 9 depicts effects of a particular anti-KIT antibody according to the present invention, mAb1, and a corresponding antibody with the same variable region sequences but an unmutated (wild type) human IgG1 sequence, mAbc, on antibody-dependent cellular cytotoxicity (ADCC) activity.

FIG. 10 depicts effects of a particular anti-KIT antibody according to the present invention, mAb1, on specific cytokine production. The conditions shown for each bar graph are, from left to right: PHA, LPS, huIgG1 (soluble), mAb1 0.02 nM (soluble), mAb1 0.2 nM (soluble), mAb1 40 nM (soluble), mAb1 0.02 nM (dry coated), mAb1 0.2 nM (dry coated), and mAb1 40 nM (dry coated).

FIG. 11 depicts a schematic illustrating the roles of KIT signaling in mast cells and the action of mAb1 on the KIT receptor.

FIGS. 12A-12D show that a single dose of mAb1 resulted in a rapid and durable response with a 95% complete response (CR) rate in patients with chronic inducible urticaria (CIndU). 10/10 cold urticaria (ColdU) patients achieved CR (FIG. 12A). 8/9 symptomatic dermographism (SD) patients achieved CR and 1/9 SD patients achieved partial response (PR) (FIG. 12B). CR=negative provocation test at ≤4° C. or 0 pins; PR=improvement by 4° C. or ≥2 pins; maximum response for each patient is shown. TempTest® results over time in ColdU patients are shown in FIG. 12C. Among completed ColdU patients (n=8), CR was sustained for a median duration of 77 days (FIG. 12C). FricTest® results over time in SD patients are shown in FIG. 12D. Among completed SD patients (n=6), CR was sustained for a median duration of 57 days (FIG. 12D).

FIGS. 13A-13B show an overall disease improvement as evidenced by physician's global assessment (Phys-GA) and patient's global assessment (Pat-GA). Phys-GA and Pat-GA assess disease severity using a Likert scale of 0-3, where 0 is none and 3 is severe.

FIGS. 14A-14D show that mAb1 treatment markedly depleted skin mast cells and serum tryptase. FIG. 14A shows that mAb1 reduced skin mast cell number (n=14, * means p<0.05, ** means p<0.01, *** means p<0.001, and **** means p<0.0001). FIG. 14B shows that mAb1 reduced serum tryptase below detection in all patients (tryptase values below assay limit of quantitation (LLoQ=1 ng/mL) was normalized to 0). FIG. 14C shows the mast cell and tryptase kinetics. FIG. 14D shows that skin mast cell numbers correlated with serum tryptase levels (p<0.0001; R²=0.45)).

FIGS. 15A-15D show that the kinetics for skin mast cell and tryptase depletion mirrored decreases in provocation thresholds. FIG. 15A shows the mast cell kinetics and the TempTest® results over time in ColdU patients. FIG. 15B shows the mast cell kinetics and the FricTest® results over time in SD patients. FIG. 15C shows the tryptase kinetics and the TempTest® results over time in ColdU patients (tryptase values below LLoQ were normalized to 0; critical temperature threshold values below 4° C. (negative test) was assigned a value of 3° C.). FIG. 15D shows the tryptase kinetics and the FricTest® results over time in SD patients.

FIGS. 16A-16D show that hematology parameters generally remained within the normal ranges and that mild, transient, and asymptomatic decreases in hemoglobin and white blood cell (WBC) parameters were noted. FIG. 16A shows the level of hemoglobin (HgB) over time. FIG. 16B shows the WBC count over time. FIG. 16C shows the platelet count over time. FIG. 16D shows the absolute neutrophil count (ANC) over time. In each graph, shaded area represents the corresponding normal range.

### 5. DETAILED DESCRIPTION

Provided herein are methods of protecting against, treating, or managing chronic prurigo, including prurigo nodularis, in a subject in need thereof, comprising administering to a subject in need thereof a therapeutically effective amount of a human KIT inhibitor. The human KIT inhibitor can be any inhibitor that inhibits the activity or expression level of a human KIT protein (e.g., a human KIT protein comprising the amino acid sequence of SEQ ID NO: 1). In specific embodiments, the human KIT inhibitor is a biologic. In specific embodiments, the human KIT inhibitor is an antibody which immunospecifically binds to human KIT, or an antigen-binding fragment thereof. In specific embodiments, the human KIT inhibitor is a small molecule inhibitor. In specific embodiments, the human KIT inhibitor is an oligonucleotide such as an aptamer, an shRNA, miRNA, siRNA, or antisense DNA.

In one aspect, provided herein is a method of protecting against, treating, or managing chronic prurigo in a subject, comprising administering to a subject in need thereof a therapeutically effective amount of an antibody which immunospecifically binds to human KIT (e.g., a KIT polypeptide containing a human KIT D4 or D5 domain, such as a human KIT protein comprising the amino acid sequence of SEQ ID NO: 1), or an antigen binding fragment thereof.

In another aspect, provided herein is a method of protecting against chronic prurigo in a subject, comprising administering to a subject in need thereof a therapeutically effective amount of an antibody which immunospecifically binds to human KIT (e.g., a KIT polypeptide containing a human KIT D4 or D5 domain, such as a human KIT protein comprising the amino acid sequence of SEQ ID NO: 1), or an antigen binding fragment thereof.

In another aspect, provided herein is a method of treating chronic prurigo in a subject, comprising administering to a subject in need thereof a therapeutically effective amount of an antibody which immunospecifically binds to human KIT (e.g., a KIT polypeptide containing a human KIT D4 or D5 domain, such as a human KIT protein comprising the amino acid sequence of SEQ ID NO: 1), or an antigen binding fragment thereof.

In another aspect, provided herein is a method of managing chronic prurigo in a subject, comprising administering to a subject in need thereof a therapeutically effective amount of an antibody which immunospecifically binds to human KIT (e.g., a KIT polypeptide containing a human KIT D4 or D5 domain, such as a human KIT protein comprising the amino acid sequence of SEQ ID NO: 1), or an antigen binding fragment thereof.

In specific embodiments, the chronic prurigo is prurigo nodularis. In specific embodiments, the chronic prurigo is popular prurigo. In specific embodiments, the chronic prurigo is nodular prurigo. In specific embodiments, the chronic prurigo is plaque prurigo. In specific embodiments, the chronic prurigo is umbilicated prurigo. In specific embodiments, the chronic prurigo is linear prurigo. In some embodiments, the subject shows a monomorphic phenotype of lesions. In other embodiments, the subject shows a polymorphic phenotype of lesions.

In certain embodiments, an anti-KIT antibody, antigen binding fragment thereof, or conjugate described herein is administered to a subject to treat, manage, prevent, or protect against both chronic prurigo (e.g., prurigo nodularis) and urticaria (e.g., chronic urticaria, including chronic spontaneous urticaria, chronic idiopathic urticaria and chronic induced urticaria (i.e., chronic inducible urticaria (CIndU)), such as cold urticaria (ColdU), symptomatic dermographism (SD), cholinergic urticaria, heat urticaria, delayed pressure urticaria, solar urticaria, vibratory urticaria, contact urticaria, or aquagenic urticaria). In a specific embodiment, an anti-KIT antibody, antigen binding fragment thereof, or conjugate described herein is administered to a subject to treat, manage, prevent, or protect against both chronic prurigo (e.g., prurigo nodularis) and chronic induced urticaria (i.e., CIndU), such as ColdU, SD, heat urticaria, delayed pressure urticaria, solar urticaria, vibratory urticaria, contact urticaria, or aquagenic urticaria). In a specific embodiment, an anti-KIT antibody, antigen binding fragment thereof, or conjugate described herein is administered to a subject to treat, manage, prevent, or protect against both chronic prurigo (e.g., prurigo nodularis) and SD. In a specific embodiment, an anti-KIT antibody, antigen binding fragment thereof, or conjugate described herein is administered to a subject to treat, manage, prevent, or protect against both prurigo nodularis and SD.

In various embodiments, the subject having chronic prurigo has failed one or more prior treatments for the chronic prurigo. In certain embodiments, the one or more prior treatments comprise at least one standard of care therapy for the chronic prurigo. In certain embodiments, the one or more prior treatments are all standard of care therapies for the chronic prurigo. In certain embodiments, the subject having chronic prurigo has failed a treatment with an IL-4R inhibitor such as an anti-IL-4R antibody, e.g., dupilumab (Dupixent®). In certain embodiments, the subject having chronic prurigo has failed a treatment with an IL-31 receptor alpha inhibitor such as an anti-IL-31 receptor alpha antibody, e.g., nemolizumab. In certain embodiments, the subject having chronic prurigo has failed a treatment with a Janus kinase 1 inhibitor, e.g., INCB054707 or abrocitinib. In certain embodiments, the subject having chronic prurigo has failed a treatment with a Neurokinin-1 (NK1) receptor antagonist, e.g., serlopitant. In certain embodiments, the subject having chronic prurigo has failed a treatment with a PDE4 and/or TNF-α, inhibitor, e.g., apremilast. In certain embodiments, the subject having chronic prurigo has failed a treatment with an OSMR 3 inhibitor such as anti-OSMRJ antibody, e.g., vixarelimab. In certain embodiments, the subject having chronic prurigo has failed one, two, three, or more of the treatments described above for the chronic prurigo.

A subject is considered to have failed a treatment for chronic prurigo if the chronic prurigo is refractory to the treatment, resistant to the treatment, relapses after the treatment, and/or if the subject has discontinued the treatment due to intolerance of the treatment.

In various embodiments, the chronic prurigo is refractory to one or more prior treatments for the chronic prurigo. In certain embodiments, the one or more prior treatments comprise at least one standard of care therapy for the chronic prurigo. In certain embodiments, the one or more prior treatments are all standard of care therapies for the chronic prurigo. In certain embodiments, the chronic prurigo is refractory to a treatment with an IL-4R inhibitor such as an anti-IL-4R antibody, e.g., dupilumab (Dupixent®). In certain embodiments, the chronic prurigo is refractory to a treatment with an IL-31 receptor alpha inhibitor such as an anti-IL-31 receptor alpha antibody, e.g., nemolizumab. In certain embodiments, the chronic prurigo is refractory to a treatment with a Janus kinase 1 inhibitor, e.g., INCB054707 or abrocitinib. In certain embodiments, the chronic prurigo is refractory to a treatment with a Neurokinin-1 (NK1) receptor antagonist, e.g., serlopitant. In certain embodiments, the chronic prurigo is refractory to a treatment with a PDE4 and/or TNF-α inhibitor, e.g., apremilast. In certain embodiments, the chronic prurigo is refractory to a treatment with an OSMR 3 inhibitor such as anti-OSMRJ antibody, e.g., vixarelimab. In certain embodiments, the chronic prurigo is refractory to one, two, three, or more of the treatments described above for the chronic prurigo.

In various embodiments, the chronic prurigo is resistant to one or more prior treatments for the chronic prurigo. In certain embodiments, the one or more prior treatments comprise at least one standard of care therapy for the chronic prurigo. In certain embodiments, the one or more prior treatments are all standard of care therapies for the chronic prurigo. In certain embodiments, the chronic prurigo is resistant to a treatment with an IL-4R inhibitor such as an anti-IL-4R antibody, e.g., dupilumab (Dupixent®). In certain embodiments, the chronic prurigo is resistant to a treatment with an IL-31 receptor alpha inhibitor such as an anti-IL-31 receptor alpha antibody, e.g., nemolizumab. In certain embodiments, the chronic prurigo is resistant to a treatment with a Janus kinase 1 inhibitor, e.g., INCB054707 or abrocitinib. In certain embodiments, the chronic prurigo is resistant to a treatment with a Neurokinin-1 (NK1) receptor antagonist, e.g., serlopitant. In certain embodiments, the chronic prurigo is resistant to a treatment with a PDE4 and/or TNF-α inhibitor, e.g., apremilast. In certain embodiments, the chronic prurigo is resistant to a treatment with an OSMRJ inhibitor such as anti-OSMR 3 antibody, e.g., vixarelimab. In certain embodiments, the chronic prurigo is resistant to one, two, three, or more of the treatments described above for the chronic prurigo.

In various embodiments, the chronic prurigo is both refractory and resistant to one or more prior treatments for the chronic prurigo. In certain embodiments, the one or more prior treatments comprise at least one standard of care therapy for the chronic prurigo. In certain embodiments, the one or more prior treatments are all standard of care therapies for the chronic prurigo. In certain embodiments, the chronic prurigo is both refractory and resistant to a treatment with an IL-4R inhibitor such as an anti-IL-4R antibody, e.g., dupilumab (Dupixent®). In certain embodiments, the chronic prurigo is both refractory and resistant to a treatment with an IL-31 receptor alpha inhibitor such as an anti-IL-31 receptor alpha antibody, e.g., nemolizumab. In certain embodiments, the chronic prurigo is both refractory and resistant to a treatment with a Janus kinase 1 inhibitor, e.g., INCB054707 or abrocitinib. In certain embodiments, the chronic prurigo is both refractory and resistant to a treatment with a Neurokinin-1 (NK1) receptor antagonist, e.g., serlopitant. In certain embodiments, the chronic prurigo is both refractory and resistant to a treatment with a PDE4 and/or TNF-α inhibitor, e.g., apremilast. In certain embodiments, the chronic prurigo is both refractory and resistant to a treatment with an OSMRJ inhibitor such as anti-OSMRJ antibody, e.g., vixarelimab. In certain embodiments, the chronic prurigo is both refractory and resistant to one, two, three, or more of the treatments described above for the chronic prurigo.

In various embodiments, the chronic prurigo has relapsed after one or more prior treatments for the chronic prurigo. In certain embodiments, the one or more prior treatments comprise at least one standard of care therapy for the chronic prurigo. In certain embodiments, the one or more prior treatments are all standard of care therapies for the chronic prurigo. In certain embodiments, the chronic prurigo has relapsed after a treatment with an IL-4R inhibitor such as an anti-IL-4R antibody, e.g., dupilumab (Dupixent®). In certain embodiments, the chronic prurigo has relapsed after a treatment with an IL-31 receptor alpha inhibitor such as an anti-IL-31 receptor alpha antibody, e.g., nemolizumab. In certain embodiments, the chronic prurigo has relapsed after a treatment with a Janus kinase 1 inhibitor, e.g., INCB054707 or abrocitinib. In certain embodiments, the chronic prurigo has relapsed after a treatment with a Neurokinin-1 (NK1) receptor antagonist, e.g., serlopitant. In certain embodiments, the chronic prurigo has relapsed after a treatment with a PDE4 and/or TNF-α inhibitor, e.g., apremilast. In certain embodiments, the chronic prurigo has relapsed after a treatment with an OSMR 3 inhibitor such as anti-OSMR 3 antibody, e.g., vixarelimab. In certain embodiments, the chronic prurigo has relapsed after one, two, three, or more of the treatments described above for the chronic prurigo.

In various embodiments, the subject having chronic prurigo has discontinued one or more prior treatments for the chronic prurigo due to intolerance of the treatment(s). In certain embodiments, the one or more prior treatments comprise at least one standard of care therapy for the chronic prurigo. In certain embodiments, the one or more prior treatments are all standard of care therapies for the chronic prurigo. In certain embodiments, the subject having chronic prurigo has discontinued a treatment with an IL-4R inhibitor such as an anti-IL-4R antibody, e.g., dupilumab (Dupixent®), due to intolerance of the treatment. In certain embodiments, the subject having chronic prurigo has discontinued a treatment with an IL-31 receptor alpha inhibitor such as an anti-IL-31 receptor alpha antibody, e.g., nemolizumab, due to intolerance of the treatment. In certain embodiments, the subject having chronic prurigo has discontinued a treatment with a Janus kinase 1 inhibitor, e.g., INCB054707 or abrocitinib, due to intolerance of the treatment. In certain embodiments, the subject having chronic prurigo has discontinued a treatment with a Neurokinin-1 (NK1) receptor antagonist, e.g., serlopitant, due to intolerance of the treatment. In certain embodiments, the subject having chronic prurigo has discontinued a treatment with a PDE4 and/or TNF-α inhibitor, e.g., apremilast, due to intolerance of the treatment. In certain embodiments, the subject having chronic prurigo has discontinued a treatment with an OSMRJ inhibitor such as anti-OSMRJ antibody, e.g., vixarelimab, due to intolerance of the treatment. In certain embodiments, the subject having chronic prurigo has discontinued one, two, three, or more of the treatments described above for the chronic prurigo due to intolerance of the treatment(s).

In various embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed one or more prior treatments for the urticaria. In certain embodiments, the one or more prior treatments comprise at least one standard of care therapy for the urticaria. In certain embodiments, the one or more prior treatments are all standard of care therapies for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed an antihistamine treatment(s) for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed an H1-antihistamine treatment(s) for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed an H2-antihistamine treatment(s) for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed both H1- and H2-antihistamine treatments for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment(s) with one or more leukotriene receptor antagonists for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment(s) with one or more immunomodulators or anti-inflammatory agents for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with an IgE inhibitor such as an anti-IgE antibody, e.g., omalizumab (Xolair®) or ligelizumab, for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with an IL-4R inhibitor such as an anti-IL-4R antibody, e.g., dupilumab (Dupixent®), for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with an IL-5R inhibitor such as an anti-IL-5R antibody, e.g., benralizumab (Fasenra®), for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with an IL-5 inhibitor such as an anti-IL-S antibody, e.g., mepolizumab, for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with a Siglec 8 inhibitor such as an anti-Siglec 8 antibody, e.g., lirentelimab, for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with a TSLP or TSLPR inhibitor such as an anti-TSLP or anti-TSLPR antibody, e.g., tezepelumab (Terspire), for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with a CSaR inhibitor such as an anti-CSaR antibody, e.g., avdoralimab, for the urticaria. In specific embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment with a CD200R inhibitor such as an anti-CD200R antibody, e.g., LY3454738, for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed a treatment(s) with one or more Bruton's Tyrosine Kinase (BTK) inhibitors, e.g., remibrutinib and/or rilzabrutinib, for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed: (1) an antihistamine treatment(s) (e.g., H1- and/or H2-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Terspire™), a C5aR inhibitor such as an anti-05aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib, for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Terspire™), a C5aR inhibitor such as an anti-C5aR antibody, e.g., avdoralimab, and/or a CD200R inhibitor such as an anti-CD200R antibody, e.g., LY3454738), for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed: (1) an antihistamine treatment(s) (e.g., H1- and/or H2-antihistamine treatment(s)), (2) a treatment with an IgE inhibitor such as an anti-IgE antibody, e.g., omalizumab (Xolair®) or ligelizumab, and (3) a treatment with an IL-4R inhibitor such as an anti-IL-4R antibody, e.g., dupilumab (Dupixent®), for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the subject having the urticaria (e.g., chronic inducible urticaria) has failed: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab, for the urticaria. In certain embodiments wherein an anti-KIT antibody, antigen binding fragment thereof, or conjugate described herein is administered to a subject to treat or manage both chronic prurigo and urticaria (e.g., chronic inducible urticaria), the suejct having the urticaria (e.g., chronic inducible urticaria) has failed: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®), for the urticaria.

A subject is considered to have failed a treatment for urticaria (e.g., chronic inducible urticaria) if the urticaria (e.g., chronic inducible urticaria) is refractory to the treatment, resistant to the treatment, relapses after the treatment, and/or if the subject has discontinued the treatment due to intolerance of the treatment. In a specific embodiment, a subject having both chronic prurigo and urticaria (e.g., chronic inducible urticaria) has an urticaria (e.g., chronic inducible urticaria) that is refractory to one or more prior treatments for the urticaria, which can be one or more treatments for urticaria that are described above. In a specific embodiment, a subject having both chronic prurigo and urticaria (e.g., chronic inducible urticaria) has an urticaria (e.g., chronic inducible urticaria) that is resistant to one or more prior treatments for the urticaria, which can be one or more treatments for urticaria that are described above. In a specific embodiment, a subject having both chronic prurigo and urticaria (e.g., chronic inducible urticaria) has an urticaria (e.g., chronic inducible urticaria) that is both refractory and resistant to one or more prior treatments for the urticaria, which can be one or more treatments for urticaria that are described above. In a specific embodiment, a subject having both chronic prurigo and urticaria (e.g., chronic inducible urticaria) has an urticaria (e.g., chronic inducible urticaria) that has relapsed after one or more prior treatments for the urticaria, which can be one or more treatments for urticaria that are described above. In a specific embodiment, a subject having both chronic prurigo and urticaria (e.g., chronic inducible urticaria) has discontinued one or more prior treatments for the urticaria, which can be one or more treatments for urticaria that are described above.

In various embodiments wherein an anti-KIT antibody, antigen binding fragment thereof, or conjugate described herein is administered to a subject to treat or manage both chronic prurigo and urticaria (e.g., chronic inducible urticaria), the subject has failed one or more prior treatments for the chronic prurigo, which can be one or more treatments for chronic prurigo that are described above, and the subject also has failed one or more prior treatments for the urticaria (e.g., chronic inducible urticaria), which can be one or more treatments for urticaria that are described above.

In various embodiments, an anti-KIT antibody, antigen binding fragment thereof, or conjugate described herein is administered to a subject having chronic prurigo whose chronic prurigo remains symptomatic despite one or more prior treatments for the chronic prurigo. In one embodiment, an anti-KIT antibody, antigen binding fragment thereof, or conjugate described herein is administered to a subject having chronic prurigo whose chronic prurigo remains symptomatic despite treatment with an IL-4R inhibitor such as an anti-IL-4R antibody, e.g., dupilumab (Dupixent®). In one embodiment, an anti-KIT antibody, antigen binding fragment thereof, or conjugate described herein is administered to a subject having chronic prurigo whose chronic prurigo remains symptomatic despite treatment with an IL-31 receptor alpha inhibitor such as an anti-IL-31 receptor alpha antibody, e.g., nemolizumab. In one embodiment, an anti-KIT antibody, antigen binding fragment thereof, or conjugate described herein is administered to a subject having chronic prurigo whose chronic prurigo remains symptomatic despite treatment with a Janus kinase 1 inhibitor, e.g., INCB054707 or abrocitinib. In one embodiment, an anti-KIT antibody, antigen binding fragment thereof, or conjugate described herein is administered to a subject having chronic prurigo whose chronic prurigo remains symptomatic despite treatment with a Neurokinin-1 (NK1) receptor antagonist, e.g., serlopitant. In one embodiment, an anti-KIT antibody, antigen binding fragment thereof, or conjugate described herein is administered to a subject having chronic prurigo whose chronic prurigo remains symptomatic despite treatment with a PDE4 and/or TNF-α inhibitor, e.g., apremilast. In one embodiment, an anti-KIT antibody, antigen binding fragment thereof, or conjugate described herein is administered to a subject having chronic prurigo whose chronic prurigo remains symptomatic despite treatment with an OSMRβ inhibitor such as anti-OSMRβ antibody, e.g., vixarelimab.

In various embodiments, an anti-KIT antibody, antigen binding fragment thereof, or conjugate described herein is administered to a subject having both chronic prurigo and urticaria (e.g., chronic inducible urticaria) whose urticaria (e.g., chronic inducible urticaria) remains symptomatic despite treatment with one or more prior treatments for the urticaria, which can be one or more treatments for urticaria that are described above.

In various embodiments, an anti-KIT antibody, antigen binding fragment thereof, or conjugate described herein is administered to a subject having both chronic prurigo and urticaria (e.g., chronic inducible urticaria) whose chronic prurigo remains symptomatic despite treatment with one or more prior treatments for the chronic prurigo, which can be one or more treatments for chronic prurigo that are described above, and whose urticaria (e.g., chronic inducible urticaria) remains symptomatic despite treatment with one or more prior treatments for the urticaria, which can be one or more treatments for urticaria that are described above.

In various embodiments, an anti-KIT antibody, antigen binding fragment thereof, or conjugate described herein is administered to a subject to treat or manage chronic prurigo following one or more prior treatments for the chronic prurigo, wherein the subject has failed said one or more prior treatments for the chronic prurigo. In one embodiment, an anti-KIT antibody, antigen binding fragment thereof, or conjugate described herein is administered to a subject to treat or manage chronic prurigo following treatment with an IL-4R inhibitor such as an anti-IL-4R antibody, e.g., dupilumab (Dupixent®), for the chronic prurigo, wherein the subject has failed said treatment with an IL-4R inhibitor. In one embodiment, an anti-KIT antibody, antigen binding fragment thereof, or conjugate described herein is administered to a subject to treat or manage chronic prurigo following treatment with an IL-31 receptor alpha inhibitor such as an anti-IL-31 receptor alpha antibody, e.g., nemolizumab, for the chronic prurigo, wherein the subject has failed said treatment with an IL-31 receptor alpha inhibitor. In one embodiment, an anti-KIT antibody, antigen binding fragment thereof, or conjugate described herein is administered to a subject to treat or manage chronic prurigo following treatment with a Janus kinase 1 inhibitor, e.g., INCB054707 or abrocitinib, for the chronic prurigo, wherein the subject has failed said treatment with a Janus kinase 1 inhibitor. In one embodiment, an anti-KIT antibody, antigen binding fragment thereof, or conjugate described herein is administered to a subject to treat or manage chronic prurigo following treatment with a Neurokinin-1 (NK1) receptor antagonist, e.g., serlopitant, for the chronic prurigo, wherein the subject has failed said treatment with a NK1 receptor antagonist. In one embodiment, an anti-KIT antibody, antigen binding fragment thereof, or conjugate described herein is administered to a subject to treat or manage chronic prurigo following treatment with a PDE4 and/or TNF-α inhibitor, e.g., apremilast, for the chronic prurigo, wherein the subject has failed said treatment with a PDE4 and/or TNF-α inhibitor. In one embodiment, an anti-KIT antibody, antigen binding fragment thereof, or conjugate described herein is administered to a subject to treat or manage chronic prurigo following treatment with an OSMRβ inhibitor such as anti-OSMRβ antibody, e.g., vixarelimab, for the chronic prurigo, wherein the subject has failed said treatment with an OSMRβ inhibitor. In one embodiment, an anti-KIT antibody, antigen binding fragment thereof, or conjugate described herein is administered to a subject to treat or manage chronic prurigo following one, two, three, or more of the treatments described above for the chronic prurigo, wherein the subject has failed said one, two, three, or more treatments.

In various embodiments, an anti-KIT antibody, antigen binding fragment thereof, or conjugate described herein is administered to a subject to treat or manage chronic prurigo following one or more prior treatments for the chronic prurigo, wherein the chronic prurigo is refractory to said one or more prior treatments for the chronic prurigo. In one embodiment, an anti-KIT antibody, antigen binding fragment thereof, or conjugate described herein is administered to a subject to treat or manage chronic prurigo following treatment with an IL-4R inhibitor such as an anti-IL-4R antibody, e.g., dupilumab (Dupixent®), for the chronic prurigo, wherein the chronic prurigo is refractory to said treatment with an IL-4R inhibitor. In one embodiment, an anti-KIT antibody, antigen binding fragment thereof, or conjugate described herein is administered to a subject to treat or manage chronic prurigo following treatment with an IL-31 receptor alpha inhibitor such as an anti-IL-31 receptor alpha antibody, e.g., nemolizumab, for the chronic prurigo, wherein the chronic prurigo is refractory to said treatment with an IL-31 receptor alpha inhibitor. In one embodiment, an anti-KIT antibody, antigen binding fragment thereof, or conjugate described herein is administered to a subject to treat or manage chronic prurigo following treatment with a Janus kinase 1 inhibitor, e.g., INCB054707 or abrocitinib, for the chronic prurigo, wherein the chronic prurigo is refractory to said treatment with a Janus kinase 1 inhibitor. In one embodiment, an anti-KIT antibody, antigen binding fragment thereof, or conjugate described herein is administered to a subject to treat or manage chronic prurigo following treatment with a Neurokinin-1 (NK1) receptor antagonist, e.g., serlopitant, for the chronic prurigo, wherein the chronic prurigo is refractory to said treatment with a NK1 receptor antagonist. In one embodiment, an anti-KIT antibody, antigen binding fragment thereof, or conjugate described herein is administered to a subject to treat or manage chronic prurigo following treatment with a PDE4 and/or TNF-α inhibitor, e.g., apremilast, for the chronic prurigo, wherein the chronic prurigo is refractory to said treatment with a PDE4 and/or TNF-α inhibitor. In one embodiment, an anti-KIT antibody, antigen binding fragment thereof, or conjugate described herein is administered to a subject to treat or manage chronic prurigo following treatment with an OSMRβ inhibitor such as anti-OSMRβ antibody, e.g., vixarelimab, for the chronic prurigo, wherein the chronic prurigo is refractory to said treatment with an OSMRβ inhibitor. In one embodiment, an anti-KIT antibody, antigen binding fragment thereof, or conjugate described herein is administered to a subject to treat or manage chronic prurigo following one, two, three, or more of the treatments described above for the chronic prurigo, wherein the chronic prurigo is refractory to said one, two, three, or more treatments.

In various embodiments, an anti-KIT antibody, antigen binding fragment thereof, or conjugate described herein is administered to a subject to treat or manage chronic prurigo following one or more prior treatments for the chronic prurigo, wherein the chronic prurigo is resistant to said one or more prior treatments for the chronic prurigo. In one embodiment, an anti-KIT antibody, antigen binding fragment thereof, or conjugate described herein is administered to a subject to treat or manage chronic prurigo following treatment with an IL-4R inhibitor such as an anti-IL-4R antibody, e.g., dupilumab (Dupixent®), for the chronic prurigo, wherein the chronic prurigo is resistant to said treatment with an IL-4R inhibitor. In one embodiment, an anti-KIT antibody, antigen binding fragment thereof, or conjugate described herein is administered to a subject to treat or manage chronic prurigo following treatment with an IL-31 receptor alpha inhibitor such as an anti-IL-31 receptor alpha antibody, e.g., nemolizumab, for the chronic prurigo, wherein the chronic prurigo is resistant to said treatment with an IL-31 receptor alpha inhibitor. In one embodiment, an anti-KIT antibody, antigen binding fragment thereof, or conjugate described herein is administered to a subject to treat or manage chronic prurigo following treatment with a Janus kinase 1 inhibitor, e.g., INCB054707 or abrocitinib, for the chronic prurigo, wherein the chronic prurigo is resistant to said treatment with a Janus kinase 1 inhibitor. In one embodiment, an anti-KIT antibody, antigen binding fragment thereof, or conjugate described herein is administered to a subject to treat or manage chronic prurigo following treatment with a Neurokinin-1 (NK1) receptor antagonist, e.g., serlopitant, for the chronic prurigo, wherein the chronic prurigo is resistant to said treatment with a NK1 receptor antagonist. In one embodiment, an anti-KIT antibody, antigen binding fragment thereof, or conjugate described herein is administered to a subject to treat or manage chronic prurigo following treatment with a PDE4 and/or TNF-α inhibitor, e.g., apremilast, for the chronic prurigo, wherein the chronic prurigo is resistant to said treatment with a PDE4 and/or TNF-α inhibitor. In one embodiment, an anti-KIT antibody, antigen binding fragment thereof, or conjugate described herein is administered to a subject to treat or manage chronic prurigo following treatment with an OSMRβ inhibitor such as anti-OSMRβ antibody, e.g., vixarelimab, for the chronic prurigo, wherein the chronic prurigo is resistant to said treatment with an OSMRβ inhibitor. In one embodiment, an anti-KIT antibody, antigen binding fragment thereof, or conjugate described herein is administered to a subject to treat or manage chronic prurigo following one, two, three, or more of the treatments described above for the chronic prurigo, wherein the chronic prurigo is resistant to said one, two, three, or more treatments.

In various embodiments, an anti-KIT antibody, antigen binding fragment thereof, or conjugate described herein is administered to a subject to treat or manage chronic prurigo following one or more prior treatments for the chronic prurigo, wherein the chronic prurigo is both refractory and resistant to said one or more prior treatments for the chronic prurigo. In one embodiment, an anti-KIT antibody, antigen binding fragment thereof, or conjugate described herein is administered to a subject to treat or manage chronic prurigo following treatment with an IL-4R inhibitor such as an anti-IL-4R antibody, e.g., dupilumab (Dupixent®), for the chronic prurigo, wherein the chronic prurigo is both refractory and resistant to said treatment with an IL-4R inhibitor. In one embodiment, an anti-KIT antibody, antigen binding fragment thereof, or conjugate described herein is administered to a subject to treat or manage chronic prurigo following treatment with an IL-31 receptor alpha inhibitor such as an anti-IL-31 receptor alpha antibody, e.g., nemolizumab, for the chronic prurigo, wherein the chronic prurigo is both refractory and resistant to said treatment with an IL-31 receptor alpha inhibitor. In one embodiment, an anti-KIT antibody, antigen binding fragment thereof, or conjugate described herein is administered to a subject to treat or manage chronic prurigo following treatment with a Janus kinase 1 inhibitor, e.g., INCB054707 or abrocitinib, for the chronic prurigo, wherein the chronic prurigo is both refractory and resistant to said treatment with a Janus kinase 1 inhibitor. In one embodiment, an anti-KIT antibody, antigen binding fragment thereof, or conjugate described herein is administered to a subject to treat or manage chronic prurigo following treatment with a Neurokinin-1 (NK1) receptor antagonist, e.g., serlopitant, for the chronic prurigo, wherein the chronic prurigo is both refractory and resistant to said treatment with a NK1 receptor antagonist. In one embodiment, an anti-KIT antibody, antigen binding fragment thereof, or conjugate described herein is administered to a subject to treat or manage chronic prurigo following treatment with a PDE4 and/or TNF-α inhibitor, e.g., apremilast, for the chronic prurigo, wherein the chronic prurigo is both refractory and resistant to said treatment with a PDE4 and/or TNF-α inhibitor. In one embodiment, an anti-KIT antibody, antigen binding fragment thereof, or conjugate described herein is administered to a subject to treat or manage chronic prurigo following treatment with an OSMRβ inhibitor such as anti-OSMRβ antibody, e.g., vixarelimab, for the chronic prurigo, wherein the chronic prurigo is both refractory and resistant to said treatment with an OSMRβ inhibitor. In one embodiment, an anti-KIT antibody, antigen binding fragment thereof, or conjugate described herein is administered to a subject to treat or manage chronic prurigo following one, two, three, or more of the treatments described above for the chronic prurigo, wherein the chronic prurigo is both refractory and resistant to said one, two, three, or more treatments.

In various embodiments, an anti-KIT antibody, antigen binding fragment thereof, or conjugate described herein is administered to a subject to treat or manage chronic prurigo following one or more prior treatments for the chronic prurigo, wherein the chronic prurigo has relapsed after said one or more prior treatments for the chronic prurigo. In one embodiment, an anti-KIT antibody, antigen binding fragment thereof, or conjugate described herein is administered to a subject to treat or manage chronic prurigo following treatment with an IL-4R inhibitor such as an anti-IL-4R antibody, e.g., dupilumab (Dupixent®), for the chronic prurigo, wherein the chronic prurigo has relapsed after said treatment with an IL-4R inhibitor. In one embodiment, an anti-KIT antibody, antigen binding fragment thereof, or conjugate described herein is administered to a subject to treat or manage chronic prurigo following treatment with an IL-31 receptor alpha inhibitor such as an anti-IL-31 receptor alpha antibody, e.g., nemolizumab, for the chronic prurigo, wherein the chronic prurigo has relapsed after said treatment with an IL-31 receptor alpha inhibitor. In one embodiment, an anti-KIT antibody, antigen binding fragment thereof, or conjugate described herein is administered to a subject to treat or manage chronic prurigo following treatment with a Janus kinase 1 inhibitor, e.g., INCB054707 or abrocitinib, for the chronic prurigo, wherein the chronic prurigo has relapsed after said treatment with a Janus kinase 1 inhibitor. In one embodiment, an anti-KIT antibody, antigen binding fragment thereof, or conjugate described herein is administered to a subject to treat or manage chronic prurigo following treatment with a Neurokinin-1 (NK1) receptor antagonist, e.g., serlopitant, for the chronic prurigo, wherein the chronic prurigo has relapsed after said treatment with a NK1 receptor antagonist. In one embodiment, an anti-KIT antibody, antigen binding fragment thereof, or conjugate described herein is administered to a subject to treat or manage chronic prurigo following treatment with a PDE4 and/or TNF-α inhibitor, e.g., apremilast, for the chronic prurigo, wherein the chronic prurigo has relapsed after said treatment with a PDE4 and/or TNF-α inhibitor. In one embodiment, an anti-KIT antibody, antigen binding fragment thereof, or conjugate described herein is administered to a subject to treat or manage chronic prurigo following treatment with an OSMRβ inhibitor such as anti-OSMRβ antibody, e.g., vixarelimab, for the chronic prurigo, wherein the chronic prurigo has relapsed after said treatment with an OSMRβ inhibitor. In one embodiment, an anti-KIT antibody, antigen binding fragment thereof, or conjugate described herein is administered to a subject to treat or manage chronic prurigo following one, two, three, or more of the treatments described above for the chronic prurigo, wherein the chronic prurigo has relapsed after said one, two, three, or more treatments.

In various embodiments, an anti-KIT antibody, antigen binding fragment thereof, or conjugate described herein is administered to a subject to treat or manage chronic prurigo following one or more prior treatments for the chronic prurigo, wherein the subject has discontinued said one or more prior treatments for the chronic prurigo due to intolerance of the treatment(s). In one embodiment, an anti-KIT antibody, antigen binding fragment thereof, or conjugate described herein is administered to a subject to treat or manage chronic prurigo following treatment with an IL-4R inhibitor such as an anti-IL-4R antibody, e.g., dupilumab (Dupixent®), for the chronic prurigo, wherein the subject has discontinued said treatment with an IL-4R inhibitor due to intolerance of the treatment. In one embodiment, an anti-KIT antibody, antigen binding fragment thereof, or conjugate described herein is administered to a subject to treat or manage chronic prurigo following treatment with an IL-31 receptor alpha inhibitor such as an anti-IL-31 receptor alpha antibody, e.g., nemolizumab, for the chronic prurigo, wherein the subject has discontinued said treatment with an IL-31 receptor alpha inhibitor due to intolerance of the treatment. In one embodiment, an anti-KIT antibody, antigen binding fragment thereof, or conjugate described herein is administered to a subject to treat or manage chronic prurigo following treatment with a Janus kinase 1 inhibitor, e.g., INCB054707 or abrocitinib, for the chronic prurigo, wherein the subject has discontinued said treatment with a Janus kinase 1 inhibitor due to intolerance of the treatment. In one embodiment, an anti-KIT antibody, antigen binding fragment thereof, or conjugate described herein is administered to a subject to treat or manage chronic prurigo following treatment with a Neurokinin-1 (NK1) receptor antagonist, e.g., serlopitant, for the chronic prurigo, wherein the subject has discontinued said treatment with a NK1 receptor antagonist due to intolerance of the treatment. In one embodiment, an anti-KIT antibody, antigen binding fragment thereof, or conjugate described herein is administered to a subject to treat or manage chronic prurigo following treatment with a PDE4 and/or TNF-α inhibitor, e.g., apremilast, for the chronic prurigo, wherein the subject has discontinued said treatment with a PDE4 and/or TNF-α inhibitor due to intolerance of the treatment. In one embodiment, an anti-KIT antibody, antigen binding fragment thereof, or conjugate described herein is administered to a subject to treat or manage chronic prurigo following treatment with an OSMRβ inhibitor such as anti-OSMRβ antibody, e.g., vixarelimab, for the chronic prurigo, wherein the subject has discontinued said treatment with an OSMRβ inhibitor due to intolerance of the treatment. In one embodiment, an anti-KIT antibody, antigen binding fragment thereof, or conjugate described herein is administered to a subject to treat or manage chronic prurigo following one, two, three, or more of the treatments described above for the chronic prurigo, wherein the subject has discontinued said one, two, three, or more treatments due to intolerance of the treatment(s).

In various embodiments, an anti-KIT antibody, antigen binding fragment thereof, or conjugate described herein is administered to a subject to treat or manage both chronic prurigo and urticaria (e.g., chronic inducible urticaria) following treatment with one or more prior treatments for the urticaria, which can be one or more treatments for urticaria that are described above, wherein the subject has failed said one or more prior treatments for the urticaria.

In various embodiments, an anti-KIT antibody, antigen binding fragment thereof, or conjugate described herein is administered to a subject to treat or manage both chronic prurigo and urticaria (e.g., chronic inducible urticaria) following treatment with one or more prior treatments for the chronic prurigo, which can be one or more treatments for chronic prurigo that are described above, wherein the subject has failed said one or more prior treatments for the chronic prurigo, and following treatment with one or more prior treatments for the urticaria, which can be one or more treatments for urticaria that are described above, wherein the subject has failed said one or more prior treatments for the urticaria.

Also, presented herein are combination therapies for the treatment of chronic prurigo which involve the administration of an anti-KIT antibody described herein (e.g., a humanized anti-KIT antibody), or an antigen-binding fragment thereof (e.g., KIT-binding fragment thereof), or an antibody conjugate thereof in combination with one or more additional therapies (e.g., a second therapeutic agent) to a subject in need thereof. In various embodiments, the combination therapies are administered to the subject at about the same time, the same day, the same week, or the same treatment cycle, or on similar or overlapping dosing schedules. In some embodiments, the combination therapies are administered to the subject simultaneously, concurrently or concomitantly. In other embodiments, the combination therapies are administered to the subject sequentially. In one embodiment, the anti-KIT antibody, antigen binding fragment thereof, or conjugate described herein is administered to the subject prior to the other therapy(ies) in the combination. In another embodiment, the anti-KIT antibody, antigen binding fragment thereof, or conjugate described herein is administered to the subject subsequent to the other therapy(ies) in the combination. The use of the term “in combination” does not restrict the order in which one or more anti-KIT antibodies and one or more additional therapies are administered to a subject. For subjects having both chronic prurigo and urticaria (e.g., chronic inducible urticaria), also presented herein are combination therapies for the treatment of urticaria (e.g., chronic inducible urticaria), as well as both combination therapies for the treatment of chronic prurigo and combination therapies for the treatment of urticaria (e.g., chronic inducible urticaria).

In various embodiments, an anti-KIT antibody, antigen binding fragment thereof, or conjugate described herein is administered to a subject having chronic prurigo in combination with one or more treatments for the chronic prurigo. In one embodiment, an anti-KIT antibody, antigen binding fragment thereof, or conjugate described herein is administered to a subject having chronic prurigo in combination with a treatment with an IL-4R inhibitor such as an anti-IL-4R antibody, e.g., dupilumab (Dupixent®), for the chronic prurigo. In one embodiment, an anti-KIT antibody, antigen binding fragment thereof, or conjugate described herein is administered to a subject having chronic prurigo in combination with a treatment with an IL-31 receptor alpha inhibitor such as an anti-IL-31 receptor alpha antibody, e.g., nemolizumab, for the chronic prurigo. In one embodiment, an anti-KIT antibody, antigen binding fragment thereof, or conjugate described herein is administered to a subject having chronic prurigo in combination with a treatment with a Janus kinase 1 inhibitor, e.g., INCB054707 or abrocitinib, for the chronic prurigo. In one embodiment, an anti-KIT antibody, antigen binding fragment thereof, or conjugate described herein is administered to a subject having chronic prurigo in combination with a treatment with Neurokinin-1 (NK1) receptor antagonist, e.g., serlopitant, for the chronic prurigo. In one embodiment, an anti-KIT antibody, antigen binding fragment thereof, or conjugate described herein is administered to a subject having chronic prurigo in combination with a treatment with PDE4 and/or TNF-α inhibitor, e.g., apremilast, for the chronic prurigo. In one embodiment, an anti-KIT antibody, antigen binding fragment thereof, or conjugate described herein is administered to a subject having chronic prurigo in combination with a treatment with an OSMRβ inhibitor such as anti-OSMRβ antibody, e.g., vixarelimab, for the chronic prurigo. In one embodiment, an anti-KIT antibody, antigen binding fragment thereof, or conjugate described herein is administered to a subject having chronic prurigo in combination with one, two, three, or more of the treatments described above for the chronic prurigo.

In various embodiments, an anti-KIT antibody, antigen binding fragment thereof, or conjugate described herein is administered to a subject having both chronic prurigo and urticaria (e.g., chronic inducible urticaria) in combination with one or more treatments for the urticaria, which can be one or more treatments for urticaria that are described above.

In various embodiments, an anti-KIT antibody, antigen binding fragment thereof, or conjugate described herein is administered to a subject having both chronic prurigo and urticaria (e.g., chronic inducible urticaria) in combination with one or more treatments for the chronic prurigo, which can be one or more treatments for chronic prurigo that are described above, and in combination with one or more treatments for the urticaria, which can be one or more treatments for urticaria that are described above.

In various embodiments, an anti-KIT antibody, antigen binding fragment thereof, or conjugate described herein is administered to a subject having chronic prurigo simultaneously, concurrently or concomitantly with one or more treatments for the chronic prurigo. In one embodiment, an anti-KIT antibody, antigen binding fragment thereof, or conjugate described herein is administered to a subject having chronic prurigo simultaneously, concurrently or concomitantly with a treatment with an IL-4R inhibitor such as an anti-IL-4R antibody, e.g., dupilumab (Dupixent®), for the chronic prurigo. In one embodiment, an anti-KIT antibody, antigen binding fragment thereof, or conjugate described herein is administered to a subject having chronic prurigo simultaneously, concurrently or concomitantly with a treatment with an IL-31 receptor alpha inhibitor such as an anti-IL-31 receptor alpha antibody, e.g., nemolizumab, for the chronic prurigo. In one embodiment, an anti-KIT antibody, antigen binding fragment thereof, or conjugate described herein is administered to a subject having chronic prurigo simultaneously, concurrently or concomitantly with a treatment with a Janus kinase 1 inhibitor, e.g., INCB054707 or abrocitinib, for the chronic prurigo. In one embodiment, an anti-KIT antibody, antigen binding fragment thereof, or conjugate described herein is administered to a subject having chronic prurigo simultaneously, concurrently or concomitantly with a treatment with Neurokinin-1 (NK1) receptor antagonist, e.g., serlopitant, for the chronic prurigo. In one embodiment, an anti-KIT antibody, antigen binding fragment thereof, or conjugate described herein is administered to a subject having chronic prurigo simultaneously, concurrently or concomitantly with a treatment with PDE4 and/or TNF-α inhibitor, e.g., apremilast, for the chronic prurigo. In one embodiment, an anti-KIT antibody, antigen binding fragment thereof, or conjugate described herein is administered to a subject having chronic prurigo simultaneously, concurrently or concomitantly with a treatment with an OSMRβ inhibitor such as anti-OSMRβ antibody, e.g., vixarelimab, for the chronic prurigo. In one embodiment, an anti-KIT antibody, antigen binding fragment thereof, or conjugate described herein is administered to a subject having chronic prurigo simultaneously, concurrently or concomitantly with one, two, three, or more of the treatments described above for the chronic prurigo.

In various embodiments, an anti-KIT antibody, antigen binding fragment thereof, or conjugate described herein is administered to a subject having both chronic prurigo and urticaria (e.g., chronic inducible urticaria) simultaneously, concurrently or concomitantly with one or more treatments for the urticaria, which can be one or more treatments for urticaria that are described above.

In various embodiments, an anti-KIT antibody, antigen binding fragment thereof, or conjugate described herein is administered to a subject having both chronic prurigo and urticaria (e.g., chronic inducible urticaria) simultaneously, concurrently or concomitantly with one or more treatments for the chronic prurigo, which can be one or more treatments for chronic prurigo that are described above, and simultaneously, concurrently or concomitantly with one or more treatments for the urticaria, which can be one or more treatments for urticaria that are described above.

In various embodiments, an anti-KIT antibody, antigen binding fragment thereof, or conjugate described herein is administered to a subject having chronic prurigo sequentially with one or more treatments for the chronic prurigo. In one embodiment, an anti-KIT antibody, antigen binding fragment thereof, or conjugate described herein is administered to a subject having chronic prurigo sequentially with a treatment with an IL-4R inhibitor such as an anti-IL-4R antibody, e.g., dupilumab (Dupixent®), for the chronic prurigo. In one embodiment, an anti-KIT antibody, antigen binding fragment thereof, or conjugate described herein is administered to a subject having chronic prurigo sequentially with a treatment with an IL-31 receptor alpha inhibitor such as an anti-IL-31 receptor alpha antibody, e.g., nemolizumab, for the chronic prurigo. In one embodiment, an anti-KIT antibody, antigen binding fragment thereof, or conjugate described herein is administered to a subject having chronic prurigo sequentially with a treatment with a Janus kinase 1 inhibitor, e.g., INCB054707 or abrocitinib, for the chronic prurigo. In one embodiment, an anti-KIT antibody, antigen binding fragment thereof, or conjugate described herein is administered to a subject having chronic prurigo sequentially with a treatment with Neurokinin-1 (NK1) receptor antagonist, e.g., serlopitant, for the chronic prurigo. In one embodiment, an anti-KIT antibody, antigen binding fragment thereof, or conjugate described herein is administered to a subject having chronic prurigo sequentially with a treatment with PDE4 and/or TNF-α inhibitor, e.g., apremilast, for the chronic prurigo. In one embodiment, an anti-KIT antibody, antigen binding fragment thereof, or conjugate described herein is administered to a subject having chronic prurigo sequentially with a treatment with an OSMR 3 inhibitor such as anti-OSMRJ antibody, e.g., vixarelimab, for the chronic prurigo. In one embodiment, an anti-KIT antibody, antigen binding fragment thereof, or conjugate described herein is administered to a subject having chronic prurigo sequentially with one, two, three, or more of the treatments described above for the chronic prurigo.

In various embodiments, an anti-KIT antibody, antigen binding fragment thereof, or conjugate described herein is administered to a subject having both chronic prurigo and urticaria (e.g., chronic inducible urticaria) sequentially with one or more treatments for the urticaria, which can be one or more treatments for urticaria that are described above.

In various embodiments, an anti-KIT antibody, antigen binding fragment thereof, or conjugate described herein is administered to a subject having both chronic prurigo and urticaria (e.g., chronic inducible urticaria) sequentially with one or more treatments for the chronic prurigo, which can be one or more treatments for chronic prurigo that are described above, and sequentially with one or more treatments for the urticaria, which can be one or more treatments for urticaria that are described above.

Further provided herein are antibodies, and antigen-binding fragments thereof, that immunospecifically bind to a human KIT (e.g., a KIT polypeptide containing a human KIT D4 or D5 domain, such as a human KIT protein comprising the amino acid sequence of SEQ ID NO: 1), which can be used in the above methods.

As used herein, “administer” or “administration” refers to the act of injecting or otherwise physically delivering a substance (e.g., a humanized anti-KIT antibody provided herein or an antigen-binding fragment thereof) to a subject or a patient (e.g., human), such as by mucosal, topical, intradermal, parenteral, intravenous, intramuscular delivery and/or any other method of physical delivery described herein or known in the art.

As used herein, the terms “effective amount” or “therapeutically effective amount” refer to an amount of a therapy (e.g., an antibody or pharmaceutical composition provided herein) which is sufficient to reduce and/or ameliorate the severity and/or duration of a given disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy (e.g., a therapy other than an anti-KIT inhibitor (e.g., an anti-KIT antibody) provided herein). In some embodiments, “effective amount” as used herein also refers to the amount of an inhibitor (e.g., an antibody) described herein to achieve a specified result, for example, reduction in the number and/or activity of mast cells, reduction in the number and/or activity of eosinophils, inhibition (e.g., partial inhibition) of a KIT biological activity of a cell, such as inhibition of cell proliferation or cell survival, or enhancement or induction of apoptosis or cell differentiation, and the like.

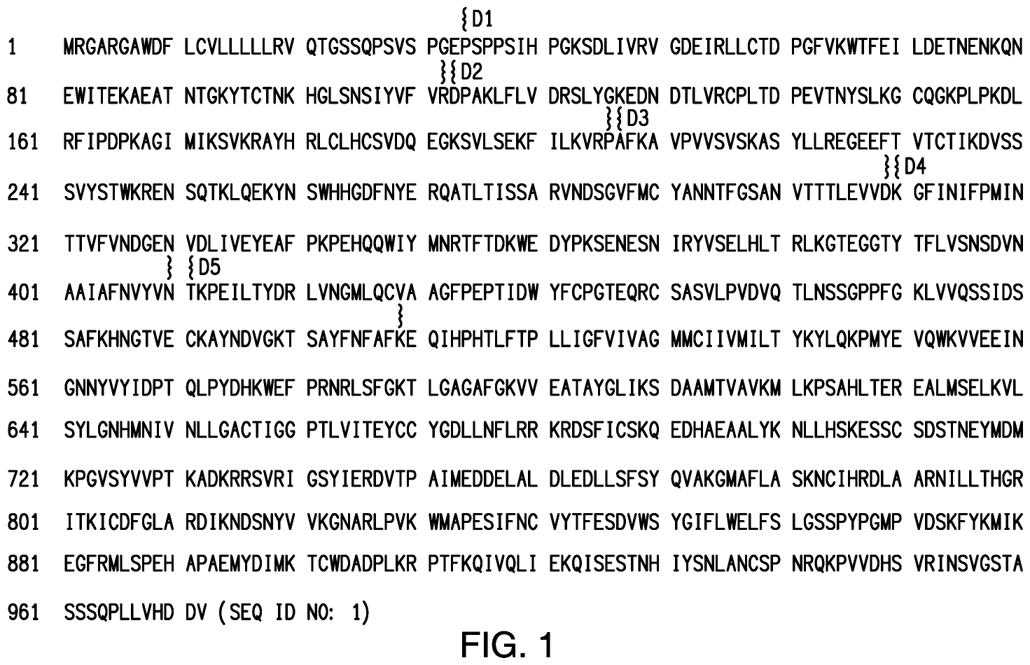

As used herein, the terms “D4 or D5 region” or “D4/D5 domain” refer to a region within a KIT polypeptide spanning the fourth Ig-like extracellular (“134”) domain, the fifth Ig-like extracellular (“D5”) domain, and the hinge region in between the D4 and D5 domains (“D4-D5 hinge region”), of KIT, in the following order from the amino terminus to the carboxyl terminus: D4, D4-D5 hinge region, and D5. As used herein, amino acids V308 to H515 of FIG. 1 are considered an example of a D4/D5 region or domain.

As used herein, the terms “KIT” or “KIT receptor” or “KIT polypeptide” refer to any form of full-length KIT including, but not limited to, native KIT, an isoform of KIT, an interspecies KIT homolog, or a KIT variant, e.g., naturally occurring (for example, allelic or splice variant, or mutant, e.g., somatic mutant) or artificially constructed variant (for example, a recombinant or chemically modified variant). KIT is a type III receptor tyrosine kinase encoded by the c-kit gene (see, e.g., Yarden et ah, Nature, 1986, 323:226-232; Ullrich and Schlessinger, Cell, 1990, 61:203-212; Clifford et al., J. Biol. Chem., 2003, 278:31461-31464; Yarden et al., EMBO J., 1987, 6:3341-3351; Mol et al., J. Biol. Chem., 2003, 278:31461-31464). GenBank™ accession number NM 000222 provides an exemplary human KIT nucleic acid sequence. GenBank™ accession numbers NP 001087241, PI 0721, and AAC50969 provide exemplary human KIT amino acid sequences. GenBank™ accession number AAH75716 provides an exemplary murine KIT amino acid sequence. Native KIT comprises five extracellular immunoglobulin (Ig)-like domains (D1, D2, D3, D4, D5), a single transmembrane region, an inhibitory cytoplasmic juxtamembrane domain, and a split cytoplasmic kinase domain separated by a kinase insert segment (see, e.g., Yarden et al., Nature, 1986, 323:226-232; Ullrich and Schlessinger, Cell, 1990, 61:203-212; Clifford et al., J. Biol. Chem., 2003, 278:31461-31464). An exemplary amino acid sequence of the D4/D5 region of human KIT is provided in FIG. 1, at amino acid residues V308 to H515. In a specific embodiment, KIT is human KIT. In a particular embodiment, KIT can exist as a monomer, dimer, multimer, native form, or denatured form.

As used herein, the term “in combination” in the context of the administration of other therapies refers to the use of more than one therapy. The use of the term “in combination” does not restrict the order in which therapies are administered. The therapies may be administered, e.g., serially, sequentially, concurrently, or concomitantly.

As used herein, the term “chronic prurigo” means a disease characterised by the presence of both chronic pruritus (itching) and multiple localized or generalized pruriginous lesions.

As used herein, the term “prurigo nodularis” means a disease characterised by the presence of both chronic pruritus and multiple localized or generalized, elevated, firm, and nodular lesions.

As used herein, the terms “treat,” “treatment” and “treating” refer to the reduction or amelioration of the progression, severity, and/or duration of chronic prurigo resulting from the administration of one or more therapies (including, but not limited to, the administration of one or more prophylactic or therapeutic agents, such as an antibody provided herein).

As used herein, the terms “manage”, “managing,” and “management” refer to the beneficial effects that a subject derives from a therapy (e.g., a prophylactic or therapeutic agent), which does not result in a cure of chronic prurigo. In certain embodiments, a subject is administered one or more therapies (e.g., prophylactic or therapeutic agents, such as an antibody described herein) to “manage” chronic prurigo, one or more symptoms thereof, so as to prevent the progression or worsening of the disorder.

As used herein, the terms “protect against,” “impede,” or “impeding” in the context of chronic prurigo, refer to the total or partial inhibition (e.g., less than 100%, 95%, 90%, 80%, 70%, 60%, 50%, 40%, 30%, 20%, 10%, or 5%) or blockage of the development, recurrence, onset or spread of chronic prurigo, and/or symptom related thereto, resulting from the administration of a therapy or combination of therapies provided herein (e.g., a combination of prophylactic or therapeutic agents, such as an antibody described herein).

As used herein, the term “prophylactic agent” refers to any agent that can totally or partially inhibit the development, recurrence, onset or spread of chronic prurigo, and/or symptom related thereto in a subject. In certain embodiments, the term “prophylactic agent” refers to an antibody described herein. In certain other embodiments, the term “prophylactic agent” refers to an agent other than an antibody described herein. Generally, a prophylactic agent is an agent which is known to be useful to or has been or is currently being used to prevent chronic prurigo, and/or a symptom related thereto or impede the onset, development, progression and/or severity of chronic prurigo, and/or a symptom related thereto. In specific embodiments, the prophylactic agent is a human anti-KIT antibody, such as a humanized or a fully human anti-KIT monoclonal antibody.

As used herein, the term “side effects” or “adverse effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) can be harmful or uncomfortable or risky. Examples of side effects include, diarrhea, cough, gastroenteritis, wheezing, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (71g ed., 2017).

As used herein, the terms “subject” and “patient” are used interchangeably. As used herein, a subject is a mammal such as a non-primate (e.g., cows, pigs, horses, cats, dogs, goats, rabbits, rats, mice, etc.) or a primate (e.g., monkey and human), for example a human. In one embodiment, the subject is a mammal, e.g., a human, diagnosed with chronic prurigo. In another embodiment, the subject is a mammal, e.g., a human, at risk of developing chronic prurigo. In another embodiment, the subject is a non-human primate. In a specific embodiment, the subject is a human adult. In a specific embodiment, the subject is an adult human subject at least 18 years old. In a specific embodiment, the subject is a human child. In a specific embodiment, the subject is a human child between 1 year old to 18 years old. In a specific embodiment, the subject is a human between 1 year to 3 years old. In a specific embodiment, the subject is a human between 3 years to 12 years old or between 12 years to 18 years old.

As used herein, the terms “therapies” and “therapy” can refer to any protocol(s), method(s), compositions, formulations, and/or agent(s) that can be used in the prevention, protection against, treatment, management, or amelioration of a condition or disorder or symptom thereof or one or more symptoms or condition associated therewith. In certain embodiments, the terms “therapies” and “therapy” refer to drug therapy, adjuvant therapy, radiation, surgery, biological therapy, supportive therapy, and/or other therapies useful in protection against, treatment, management, prevention, or amelioration of a condition or disorder or one or more symptoms thereof or one or more symptoms or condition associated therewith. In certain embodiments, the term “therapy” refers to a therapy other than an anti-KIT antibody described herein or pharmaceutical composition thereof. In specific embodiments, an “additional therapy” and “additional therapies” refer to a therapy other than a treatment using an anti-KIT antibody described herein or pharmaceutical composition thereof. In a specific embodiment, a therapy includes the use of an anti-KIT antibody described herein as an adjuvant therapy. For example, using an anti-KIT antibody described herein in conjunction with a drug therapy, biological therapy, surgery, and/or supportive therapy.

As used herein, the term “therapeutic agent” refers to any agent that can be used in the protection against, treatment, management or amelioration of chronic prurigo and/or a symptom related thereto. In certain embodiments, the term “therapeutic agent” refers to an anti-KIT antibody described herein or an antigen-binding fragment thereof. In certain other embodiments, the term “therapeutic agent” refers to an agent other than an antibody described herein. In specific embodiments, a therapeutic agent is an agent which is known to be useful for, or has been or is currently being used for the protection against, treatment, management or amelioration of chronic prurigo or one or more symptoms related thereto.

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

It is understood that wherever aspects are described herein with the language “comprising,” otherwise analogous aspects described in terms of “consisting of” and/or “consisting essentially of” are also provided.

As used herein and unless otherwise specified, the terms “about” and “approximately” shall be construed so as to allow normal variation as judged by a person of skill in the art, such as, for example, a variation within 20% or 10% or 5%. In specific embodiments, the terms “about” and “approximately” encompass the exact value recited.

### 5.1 Antibodies

Provided herein are antibodies (e.g., anti-KIT antibodies) that specifically bind to a KIT receptor (e.g., extracellular domain of a human KIT receptor for example as set forth in SEQ ID NO: 1 or FIG. 1), or an antigen binding fragment thereof, for use in methods for preventing, treating or managing chronic prurigo.

As used herein, the terms “antibody” and “immunoglobulin” and “Ig” are terms of art and can be used interchangeably herein and refer to a molecule with an antigen binding site that immunospecifically binds an antigen.

Antibodies include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecule, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′) fragments, disulfide-linked Fvs (sdFv), anti-idiotypic (anti-Id) antibodies (including, e.g., anti-anti-Id antibodies), and epitope-binding fragments of any of the above. In certain embodiments, antibodies described herein refer to polyclonal antibody populations. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof.

As used herein, an “antigen” is a moiety or molecule that contains an epitope, and, as such, also is specifically bound by an antibody. In a specific embodiment, the antigen, to which an antibody described herein binds, is KIT (e.g., human KIT), or a fragment thereof, for example, an extracellular domain of KIT (e.g., human KIT) or a D4 region of KIT (e.g., human KIT).

As used herein, the terms “antigen binding domain,” “antigen binding region,” “antigen binding fragment,” and similar terms refer to a portion of an antibody molecule which comprises the amino acid residues that interact with an antigen and confer on the antibody molecule its specificity for the antigen (e.g., the complementarity determining regions (CDR)). The antigen binding region can be derived from any animal species, such as rodents (e.g., mouse, rat or hamster) and humans. The CDRs of an antibody molecule can be determined by any method well known to one of skill in the art. In particular, the CDRs can be determined according to the Kabat numbering system (see Kabat et al. (1991) Sequences of Proteins of Immunological Interest. (U.S. Department of Health and Human Services, Washington, D.C.) 5^{th} ed.). In certain aspects, the CDRs of an antibody can be determined according to (i) the Chothia numbering scheme, which will be referred to herein as the “Chothia CDRs” (see, e.g., Chothia and Lesk, 1987, J. Mol. Biol, 196:901-917; Al-Lazikani et al., 1997, J. Mol. Biol, 273:927-948; and U.S. Pat. No. 7,709,226); (ii) the IMGT numbering system, for example, as described in Lefranc, M.-P., 1999, The Immunologist, 7: 132-136 and Lefranc, M.-P. et al., 1999, Nucleic Acids Res., 27:209-212; (iii) the AbM numbering system, for example, as described in MacCallum et al., 1996, J. Mol. Biol., 262:732-745 and Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001); or (iv) the Contact numbering system, which is based on analysis of the available complex crystal structures (bioinf.org.uk/abs) (see, e.g., MacCallum et al., (1996) J Mol Biol 5:732-745).

As used herein, the term “constant region” or “constant domain” refers to an antibody portion, e.g., a carboxyl terminal portion of a light and/or heavy chain which is not directly involved in binding of an antibody to antigen but which exhibits or contributes to various effector functions, such as interaction with the Fc receptor. The terms refer to a portion of an immunoglobulin molecule having a generally more conserved amino acid sequence relative to an immunoglobulin variable domain.

As used herein, an “epitope” is a term in the art and refers to a localized region of an antigen to which an antibody can specifically bind. A region or a polypeptide contributing to an epitope can be contiguous amino acids of the polypeptide or an epitope can come together from two or more non-contiguous regions of the polypeptide.

As used herein, the term “heavy chain” when used in reference to an antibody refers to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄. In a specific embodiment, the heavy chain is a human heavy chain.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In a specific embodiment, molecules that immunospecifically bind to an antigen bind to the antigen with a Kₐ that is at least 2 logs, 2.5 logs, 3 logs, 4 logs or greater than the Kₐ when the molecules bind to another antigen. In another specific embodiment, molecules that immunospecifically bind to an antigen do not cross react with other proteins. In another specific embodiment, molecules that immunospecifically bind to an antigen do not cross react with other non-KIT proteins.

As used herein, an “isolated” or “purified” antibody is substantially free of cellular material or other contaminating proteins from the cell or tissue source from which the antibody is derived, or substantially free of chemical precursors or other chemicals when chemically synthesized. In a specific embodiment, the antibody or antigen binding fragment described herein is isolated.

The terms “Kabat numbering,” and like terms are recognized in the art and refer to a system of numbering amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35 (“CDR1”), amino acid positions 50 to 65 (“CDR2”), and amino acid positions 95 to 102 (“CDR3”). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the term “light chain” when used in reference to an antibody refers to any distinct types, e.g., kappa (κ) of lambda (λ) based on the amino acid sequence of the constant domains. Light chain amino acid sequences are well known in the art. In specific embodiments, the light chain is a human light chain.

As used herein, the term “monoclonal antibody” refers to an antibody obtained from a population of homogenous or substantially homogeneous antibodies, and each monoclonal antibody will typically recognize a single epitope on the antigen. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single hybridoma or other cell (e.g., host cell producing a recombinant antibody), wherein the antibody immunospecifically binds to a KIT epitope (e.g., an epitope of a D4 of human KIT) as determined, e.g., by ELISA or other antigen-binding or competitive binding assay known in the art or in the Examples provided herein. Monoclonal antibodies described herein can, for example, be made by the hybridoma method as described in Kohler et ah; Nature, 256:495 (1975) or can be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et ah, eds., John Wiley and Sons, New York). In specific embodiments, a monoclonal antibody is a monospecific antibody in that its antigen binding regions are specific for the same epitope. In further specific embodiments, a monoclonal monospecific antibody can be monovalent (having one antigen binding region) or multivalent (having more than one antigen binding regions), for example, bivalent (having two antigen binding regions).

As used herein, the term “naked antibody” refers to an antibody which is not linked, fused or conjugated to another agent or molecule (e.g., label or drug), peptide or polypeptide. In specific embodiments, a naked antibody expressed by a mammalian host cell can be glycosylated by the host cell's glycosylation machinery, for example glycosylation enzymes. In certain embodiment, a naked antibody is not glycosylated when it is expressed by a host cell which does not have its own glycosylation machinery, for example glycosylation enzymes. In certain embodiments, a naked antibody is a whole antibody, and in other embodiments, a naked antibody is an antigen binding fragment of a whole antibody, such as a Fab antibody.

As used herein, the term “polyclonal antibodies” refers to an antibody population that includes a variety of different antibodies directed to the same and to different epitopes within an antigen or antigens. Methods for producing polyclonal antibodies are known in the art (See, e.g., see, for example, Chapter 11 in: Short Protocols in Molecular Biology, (2002) 5th Ed., Ausubel et ah, eds., John Wiley and Sons, New York).

As used herein, the term “recombinant human antibody” includes human antibodies that are isolated, prepared, expressed, or created by recombinant means, such as antibodies expressed using a recombinant expression vector transfected into a host cell, antibodies isolated from a recombinant, combinatorial human antibody library, antibodies isolated from an animal (e.g., a mouse, rabbit, goat, or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves creation, e.g., via synthesis, genetic engineering of DNA sequences that encode human immunoglobulin sequences, or splicing of sequences that encode human immunoglobulins, e.g., human immunoglobulin gene sequences, to other such sequences. Such recombinant human antibodies can have variable and constant regions derived from human germline immunoglobulin sequences. In certain embodiments, the amino acid sequences of such recombinant human antibodies have been modified such thus the amino acid sequences of the VH and/or VL regions of the recombinant antibodies are sequences that, while derived from and related to human germline VH and VL sequences, do not naturally exist within the human antibody germline repertoire in vivo. As a non-limiting example, a recombinant human antibody can be obtained by assembling several human sequence fragments into a composite human sequence of a recombinant human antibody.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR).

Without wishing to be bound by any particular mechanism or theory, it is believed that the CDRs of the light and heavy chains are primarily responsible for the interaction of the antibody with antigen. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242 (“Kabat et al.”). In certain aspects, the CDRs of an antibody can be determined according to (i) the Chothia numbering scheme, which will be referred to herein as the “Chothia CDRs” (see, e.g., Chothia and Lesk, 1987, J. Mol. Biol, 196:901-917; Al-Lazikani et al., 1997, J. Mol. Biol, 273:927-948; and U.S. Pat. No. 7,709,226); (ii) the IMGT numbering system, for example, as described in Lefranc, M.-P., 1999, The Immunologist, 7:132-136 and Lefranc, M.-P. et al., 1999, Nucleic Acids Res., 27:209-212; (iii) the AbM numbering system, for example, as described in MacCallum et al., 1996, J. Mol. Biol., 262:732-745 and Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001); or (iv) the Contact numbering system, which is based on analysis of the available complex crystal structures (bioinf.org.uk/abs) (see, e.g., MacCallum et al., (1996) J Mol Biol 5:732-745). In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs). As a non-limiting example, a variable region described herein is obtained from assembling two or more fragments of human sequences into a composite human sequence.

Suitable anti-KIT antibodies for use in the methods provided herein can be selected as described herein.

In a specific aspect, anti-KIT antibodies (e.g., humanized antibodies) or antigen binding fragments thereof for use in methods for preventing, treating or managing chronic prurigo comprise a light chain variable region (“VL”) comprising VL CDRs 1-3 and a heavy chain variable region (“VH”) comprising VH CDRs 1-3 as set forth in Table 1. In a specific aspect, anti-KIT antibodies (e.g., humanized antibodies) or antigen binding fragments thereof for use in methods for preventing, treating or managing chronic prurigo comprise a light chain variable region (“VL”) comprising VL CDRs 1-3 and a heavy chain variable region (“VH”) comprising VH CDRs 1-3 as set forth in Table 2 (set 1 or set 2). In a specific aspect, anti-KIT antibodies (e.g., humanized antibodies) or antigen binding fragments thereof for use in methods for preventing, treating or managing chronic prurigo comprise a light chain variable region (“VL”) comprising VL CDRs 1-3 and a heavy chain variable region (“VH”) comprising VH CDRs 1-3 as set forth in Table 3 (AbM CDRs or Contact CDRs).

In a specific aspect, anti-KIT antibodies (e.g., humanized antibodies) or antigen binding fragments thereof for use in methods for preventing, treating or managing chronic prurigo comprise a VL comprising VL CDRs 1-3 as set forth in Table 1 (SEQ ID NOs: 2-4) and a VH comprising VH CDRs 1-3 as set forth in Table 1 (SEQ ID NOs: 5-7). In a particular embodiment, such anti-KIT antibody is a naked antibody. In a specific embodiment, such anti-KIT antibody is a bivalent monospecific antibody. In a specific embodiment, such anti-KIT antibody is a bispecific antibody. In a certain embodiment, such anti-KIT antibody is not a bispecific antibody.

TABLE 1
CDR Amino Acid Sequences

| | amino acid sequence | SEQ ID NO: |
|---|---|---|
| VL CDR1 | KASQNVRTNVA | 2 |
| VL CDR2 | SASYRYS | 3 |
| VL CDR3 | QQYNSYPRT | 4 |
| VH CDR1 | DYYIN | 5 |
| VH CDR2 | RIYPGSGNTYYNEKFKG | 6 |
| VH CDR3 | GVYYFDY | 7 |

TABLE 2
CDR Amino Acid Sequences

| | Set 1 | | Set 2 | |
|---|---|---|---|---|
| | amino acid | SEQ ID | amino acid | SEQ ID |
| | sequence | NO: | sequence | NO: |
| VL CDR1 | KASQNVRTNVA | 2 | SQNVRTN | 28 |
| VL CDR2 | SASYRYS | 3 | SAS | 29 |
| VL CDR3 | QQYNSYPRT | 4 | YNSYPR | 30 |
| VH CDR1 | GYTFTDY | 25 | GYTFTDY | 25 |
| VH CDR2 | YPGSGN | 26 | PGSG | 31 |
| VH CDR3 | GVYYFDYW | 27 | VYYFDY | 32 |

TABLE 3
CDR Amino Acid Sequences

| | AbM | | Contact | |
|---|---|---|---|---|
| | amino acid | SEQ ID | amino acid | SEQ ID |
| | sequence | NO: | sequence | NO: |
| VL CDR1 | KASQNVRTNVA | 2 | RTNVAWY | 35 |
| VL CDR2 | SASYRYS | 3 | ALIYSASYRY | 36 |
| VL CDR3 | QQYNSYPRT | 4 | QQYNSYPR | 37 |
| VH CDR1 | GYTFTDYYIN | 33 | TDYYIN | 38 |
| VH CDR2 | RIYPGSGNTY | 34 | WIARIYPGSGNTY | 39 |
| VH CDR3 | GVYYFDYW | 27 | ARGVYYFDY | 40 |

In a particular aspect, an anti-KIT antibody (e.g., humanized antibody) or antigen binding fragment thereof for use in methods for preventing, treating or managing chronic prurigo comprises:
(i) a VL comprising the amino acid sequence:
   DIVMTQSPSX_{K1}LSASVGDRVTITCKASQNVRTNVAWYQQKPGKAPKX_{K2} LIYSASYRYSGVPDRFX_{K3}GSGSGTDFTLTISSLQX_{K4}EDFAX_{K5}YX_{K6}CQQYN SYPRTFGGGTKVEIK (SEQ ID NO: 17), wherein X_{K1} to X_{K6} is any amino acid; and
(ii) a VH comprising the amino acid sequence:
   QVQLVQSGAEX_{H1}KKPGASVKX_{H2}SCKASGYTFTDYYINWVX_{H3}QAPGKG LEWIARIYPGSGNTYYNEKFKGRX_{H4}TX_{H5}TAX_{H6}KSTSTAYMX_{H7}LSSLRSE DX_{H8}AVYFCARGVYYFDYWGQGTTVTVSS (SEQ ID NO: 18), wherein X_{H1} to X_{H8} is any amino acid.

In a particular embodiment, X_{κ1} is an amino acid with an aromatic or aliphatic hydroxyl side chain, X_{κ2} is an amino acid with an aliphatic or aliphatic hydroxyl side chain X_{κ3} is an amino acid with an aliphatic hydroxyl side chain X_{K4} is an amino acid with an aliphatic hydroxyl side chain or is P, X_{K4} is an amino acid with a charged or acidic side chain, X_{K6} is an amino acid with an aromatic side chain, X_{H1} is an amino acid with an aliphatic side chain, X_{H2} is an amino acid with an aliphatic side chain X_{H3} is an amino acid with a polar or basic side chain X_{H4} is an amino acid with an aliphatic side chain X_{H5} is an amino acid with an aliphatic side chain X_{H6} is an amino acid with an acidic side chain, X_{H7} is an amino acid with an acidic or amide derivative side chain, and X_{H8} is an amino acid with an aliphatic hydroxyl side chain.

In a specific embodiment, X_{K1} is the amino acid F or S, X_{κ2} is the amino acid A or S, X_{K3} is the amino acid T or S, X_{K4} is the amino acid S or P, X_{K5} is the amino acid D or T X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

In a particular aspect, an anti-KIT antibody (e.g., humanized antibody) or antigen binding fragment thereof for use in methods for preventing, treating or managing chronic prurigo comprises:
(i) a VL comprising the amino acid sequence:
DIVMTQSPSX_{K1}LSASVGDRVTITCKASQNVRTNVAWYQQKPGKAPKX_{K2} LIYSASYRYSGVPDRFX_{K3}GSGSGTDFTLTISSLQX_{K4}EDFAX_{K5}YX_{K6}CQQYN SYPRTFGGGTKVEIK (SEQ ID NO: 17), wherein X_{K1} to X_{K6} is any amino acid; and
(ii) a VH comprising a VH CDR1, VH CDR2, and VH CDR3 comprising the amino acid sequences of SEQ ID NO: 5, SEQ ID NO: 6, and SEQ ID NO: 7, respectively.

In a particular embodiment, X_{κ1} is an amino acid with an aromatic or aliphatic hydroxyl side chain, X_{κ2} is an amino acid with an aliphatic or aliphatic hydroxyl side chain X_{κ3} is an amino acid with an aliphatic hydroxyl side chain X_{K4} is an amino acid with an aliphatic hydroxyl side chain or is P, X_{K5} is an amino acid with a charged or acidic side chain, and X_{K6} is an amino acid with an aromatic side chain.

In a specific embodiment, X_{K1} is the amino acid F or S, X_{κ2} is the amino acid A or S, X_{K3} is the amino acid T or S, X_{K4} is the amino acid S or P, X_{K5} is the amino acid D or T, and X_{K6} is the amino acid F or Y.

In a particular aspect, an anti-KIT antibody (e.g., humanized antibody) or antigen binding fragment thereof for use in methods for preventing, treating or managing chronic prurigo comprises:
(i) a VL comprising a VL CDR1, VL CDR2, and VL CDR3 having the amino acid sequences of SEQ ID NO: 2, SEQ ID NO: 3, and SEQ ID NO: 4, respectively; and
(ii) a VH comprising the amino acid sequence:
QVQLVQSGAEX_{H1}KKPGASVKX₂SCKASGYTFTDYYINWVX_{H3}QAPGKG LEWIARIYPGSGNTYYNEKFKGRX_{H4}TX_{H5}TAX_{H6}KSTSTAYMX_{H7}LSSLRSE DX_{H8}AVYFCARGVYYFDYWGQGTTVTVSS (SEQ ID NO: 18), wherein X_{H1} to X_{H8} is any amino acid.

In a particular embodiment, X_{H1} is an amino acid with an aliphatic side chain, X_{H2} is an amino acid with an aliphatic side chain X_{H3} is an amino acid with a polar or basic side chain X_{H4} is an amino acid with an aliphatic side chain X_{H5} is an amino acid with an aliphatic side chain X_{H6} is an amino acid with an acidic side chain, X_{H7} is an amino acid with an acidic or amide derivative side chain, and X_{H8} is an amino acid with an aliphatic hydroxyl side chain.

In a specific embodiment, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

In a specific aspect, anti-KIT antibodies (e.g., humanized antibodies) or antigen binding fragments thereof for use in methods for preventing, treating or managing chronic prurigo, comprise a heavy chain variable region (“VH”) comprising an amino acid sequence selected from Table 4 (SEQ ID NOs: 8-12) and/or a light chain variable region (“VL”) comprising an amino acid sequence selected from Table 5 (SEQ ID NOs: 13-16). In a particular embodiment, such anti-KIT antibody is a naked antibody. In a specific embodiment, such anti-KIT antibody is a bivalent monospecific antibody. In a specific embodiment, such anti-KIT antibody is a bispecific antibody. In a certain embodiment, such anti-KIT antibody is not a bispecific antibody.

TABLE 4
VH amino acid sequence

| | Amino Acid Sequence | SEQ ID NO: |
|---|---|---|
| VH1 | QVQLVQSGAELKKPGASVKLSCKASGYTF | 8 |
| | TDYYINWVKQAPGKGLEWIARIYPGSGNTY | |
| | YNEKFKGRATLTAEKSTSTAYMQLSSLRSE | |
| | DSAVYFCARGVYYFDYWGQGTTVTVSS | |
| VH2 | QVQLVQSGAEVKKPGASVKLSCKASGYTF | 9 |
| | TDYYINWVKQAPGKGLEWIARIYPGSGNTY | |
| | YNEKFKGRATLTAEKSTSTAYMQLSSLRSE | |
| | DTAVYFCARGVYYFDYWGQGTTVTVSS | |
| VH3 | QVQLVQSGAEVKKPGASVKLSCKASGYTF | 10 |
| | TDYYINWVRQAPGKGLEWIARIYPGSGNTY | |
| | YNEKFKGRATLTADKSTSTAYMQLSSLRSE | |
| | DTAVYFCARGVYYFDYWGQGTTVTVSS | |
| VH4 | QVQLVQSGAEVKKPGASVKVSCKASGYTF | 11 |
| | TDYYINWVRQAPGKGLEWIARIYPGSGNTY | |
| | YNEKFKGRATITADKSTSTAYMELSSLRSE | |
| | DTAVYFCARGVYYFDYWGQGTTVTVSS | |
| VH5 | QVQLVQSGAEVKKPGASVKVSCKASGYTF | 12 |
| | TDYYINWVRQAPGKGLEWIARIYPGSGNTY | |
| | YNEKFKGRVTITADKSTSTAYMELSSLRSE | |
| | DTAVYFCARGVYYFDYWGQGTTVTVSS | |

TABLE 5
VL Amino Acid Sequence

| | Amino Acid Sequence | SEQ ID NO: |
|---|---|---|
| VL1 | DIVMTQSPSFLSASVGDRVTITCKASQNVR | 13 |
| | TNVAWYQQKPGKAPKALIYSASYRYSGVP | |
| | DRFTGSGSGTDFTLTISSLQSEDFADYFCQQ | |
| | YNSYPRTFGGGTKVEIK | |
| VL2 | DIVMTQSPSSLSASVGDRVTITCKASQNVR | 14 |
| | TNVAWYQQKPGKAPKALIYSASYRYSGVP | |
| | DRFTGSGSGTDFTLTISSLQPEDFADYFCQQ | |
| | YNSYPRTFGGGTKVEIK | |
| VL3 | DIVMTQSPSSLSASVGDRVTITCKASQNVR | 15 |
| | TNVAWYQQKPGKAPKALIYSASYRYSGVP | |
| | DRFSGSGSGTDFTLTISSLQPEDFADYFCQQ | |
| | YNSYPRTFGGGTKVEIK | |
| VL4 | DIVMTQSPSSLSASVGDRVTITCKASQNVR | 16 |
| | TNVAWYQQKPGKAPKSLIYSASYRYSGVP | |
| | DRFSGSGSGTDFTLTISSLQPEDFATYYCQQ | |
| | YNSYPRTFGGGTKVEIK | |

In a specific aspect, anti-KIT antibodies (e.g., humanized antibodies) or antigen binding fragments thereof for use in methods for preventing, treating or managing chronic prurigo comprise a VH comprising the amino acid sequence of SEQ ID NO: 8, and/or a VL comprising the amino acid sequence of SEQ ID NO: 13. In one embodiment, the anti-KIT antibody or antigen binding fragment thereof comprises a VH comprising the amino acid sequence of SEQ ID NO: 8, and/or a VL comprising the amino acid sequence of SEQ ID NO: 14. In one embodiment, the anti-KIT antibody or antigen binding fragment thereof comprises a VH comprising the amino acid sequence of SEQ ID NO: 8, and/or a VL comprising the amino acid sequence of SEQ ID NO: 15. In one embodiment, the anti-KIT antibody or antigen binding fragment thereof comprises a VH comprising the amino acid sequence of SEQ ID NO: 8, and/or a VL comprising the amino acid sequence of SEQ ID NO: 16.

In one embodiment, the anti-KIT antibody or antigen binding fragment thereof comprises a VH comprising the amino acid sequence of SEQ ID NO: 9, and/or a VL comprising the amino acid sequence of SEQ ID NO: 13. In one embodiment, the anti-KIT antibody or antigen binding fragment thereof comprises a VH comprising the amino acid sequence of SEQ ID NO: 9, and/or a VL comprising the amino acid sequence of SEQ ID NO: 14. In one embodiment, the anti-KIT antibody or antigen binding fragment thereof comprises a VH comprising the amino acid sequence of SEQ ID NO: 9, and/or a VL comprising the amino acid sequence of SEQ ID NO: 15. In one embodiment, the anti-KIT antibody or antigen binding fragment thereof comprises a VH comprising the amino acid sequence of SEQ ID NO: 9, and/or a VL comprising the amino acid sequence of SEQ ID NO: 16.

In one embodiment, the anti-KIT antibody or antigen binding fragment thereof comprises a VH comprising the amino acid sequence of SEQ ID NO: 10, and/or a VL comprising the amino acid sequence of SEQ ID NO: 13. In one embodiment, the anti-KIT antibody or antigen binding fragment thereof comprises a VH comprising the amino acid sequence of SEQ ID NO: 10, and/or a VL comprising the amino acid sequence of SEQ ID NO: 14. In one embodiment, the anti-KIT antibody or antigen binding fragment thereof comprises a VH comprising the amino acid sequence of SEQ ID NO: 10, and a VL comprising the amino acid sequence of SEQ ID NO: 14. In one embodiment, the anti-KIT antibody or antigen binding fragment thereof comprises a VH comprising the amino acid sequence of SEQ ID NO: 10, and/or a VL comprising the amino acid sequence of SEQ ID NO: 15. In one embodiment, the anti-KIT antibody or antigen binding fragment thereof comprises a VH comprising the amino acid sequence of SEQ ID NO: 10, and/or a VL comprising the amino acid sequence of SEQ ID NO: 16.

In one embodiment, the anti-KIT antibody or antigen binding fragment thereof comprises a VH comprising the amino acid sequence of SEQ ID NO: 11, and/or a VL comprising the amino acid sequence of SEQ ID NO: 13. In one embodiment, the anti-KIT antibody or antigen binding fragment thereof comprises a VH comprising the amino acid sequence of SEQ ID NO: 11, and/or a VL comprising the amino acid sequence of SEQ ID NO: 14. In one embodiment, the anti-KIT antibody or antigen binding fragment thereof comprises a VH comprising the amino acid sequence of SEQ ID NO: 11, and/or a VL comprising the amino acid sequence of SEQ ID NO: 15. In one embodiment, the anti-KIT antibody or antigen binding fragment thereof comprises a VH comprising the amino acid sequence of SEQ ID NO: 11, and/or a VL comprising the amino acid sequence of SEQ ID NO: 16.

In one embodiment, the anti-KIT antibody or antigen binding fragment thereof comprises a VH comprising the amino acid sequence of SEQ ID NO: 12, and/or a VL comprising the amino acid sequence of SEQ ID NO: 13. In one embodiment, the anti-KIT antibody or antigen binding fragment thereof comprises a VH comprising the amino acid sequence of SEQ ID NO: 12, and/or a VL comprising the amino acid sequence of SEQ ID NO: 14. In one embodiment, the anti-KIT antibody or antigen binding fragment thereof comprises a VH comprising the amino acid sequence of SEQ ID NO: 12, and/or a VL comprising the amino acid sequence of SEQ ID NO: 15. In one embodiment, the anti-KIT antibody or antigen binding fragment thereof comprises a VH comprising the amino acid sequence of SEQ ID NO: 12, and/or a VL comprising the amino acid sequence of SEQ ID NO: 16.

In a specific aspect, anti-KIT antibodies (e.g., humanized antibodies) or antigen binding fragments thereof for use in methods for preventing, treating or managing chronic prurigo comprise:
(i) a VL comprising an amino acid sequence that is: at least 90% identical to SEQ ID NO: 13, at least 88% identical to SEQ ID NO: 14, at least 87% identical to SEQ ID NO: 15, or at least 84% identical to SEQ ID NO: 16; and
(ii) a VH comprising an amino acid sequence that is: at least 93% identical to SEQ ID NO: 8, at least 92% identical to SEQ ID NO: 9, at least 90% identical to SEQ ID NO: 10, at least 87% identical to SEQ ID NO: 11, or at least 86% identical to SEQ ID NO: 12.

Prior anti-KIT antibodies have been found to induce degranulation of FcgRI-expressing human mast cells and/or to show Fc receptor-dependent KIT agonist activity, which may give rise to undesirable infusion-related reactions (IRRs) among other adverse effects.

In various embodiments, an anti-KIT antibody or antigen binding fragment used in the methods described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain). Preferably, an anti-KIT antibody or antigen binding fragment used in the methods described herein has reduced Fc receptor binding activity (particularly reduced FcγR binding activity), does not induce degranulation of FcgRI-expressing human mast cells, and/or show Fc receptor-dependent KIT agonist activity. In certain embodiments, one or more of these properties of the anti-KIT antibody or antigen binding fragment result from the modified (e.g., mutated) Fc region or domain.

In specific embodiments, an anti-KIT antibody or antigen binding fragment used in the methods described herein has reduced Fc receptor binding activity (particularly reduced FcγR binding activity). In specific embodiments, an anti-KIT antibody or antigen binding fragment used in the methods described herein does not have significant Fc receptor (particularly FcγR) binding activity. In specific embodiments, an anti-KIT antibody or antigen binding fragment used in the methods described herein has no detectable Fc receptor (particularly FcγR) binding activity. In particular embodiments, an anti-KIT antibody or antigen binding fragment used in the methods described herein has at least 10% less, at least 20% less, at least 30% less, at least 40% less, at least 50% less, at least 60% less, at least 70% less, at least 80% less, at least 90% less, at least 95% less, or at least 99% less Fc receptor (particularly FcγR) binding activity compared to an appropriate control antibody or antigen binding fragment. When an anti-KIT antibody or antigen binding fragment used in the methods described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain), in preferred embodiments the appropriate control antibody or antigen binding fragment is an antibody or antigen binding fragment having the same VH and VL but with a wild-type (unmodified) Fc region or domain of the same isotype. In particular embodiments, an anti-KIT antibody or antigen binding fragment used in the methods described herein comprises a modified (e.g., mutated) human IgG1 Fc region or domain and has at least 10% less, at least 20% less, at least 30% less, at least 40% less, at least 50% less, at least 60% less, at least 70% less, at least 80% less, at least 90% less, at least 95% less, or at least 99% less Fc receptor (particularly FcγR) binding activity compared to a corresponding antibody or antigen binding fragment having the same VH and VL but with a wild-type (unmodified) human IgG1 Fc region or domain.

In specific embodiments, an anti-KIT antibody or antigen binding fragment used in the methods described herein does not induce significant degranulation of FcgRI-expressing human mast cells (e.g., as determined, for example, by % release of beta-hexosaminidase from human mast cells in culture (e.g., in presence of IFN gamma)). In specific embodiments, an anti-KIT antibody or antigen binding fragment used in the methods described herein does not induce detectable degranulation of FcgRI-expressing human mast cells (e.g., as determined, for example, by % release of beta-hexosaminidase from human mast cells in culture (e.g., in presence of IFN gamma)). In particular embodiments, an anti-KIT antibody or antigen binding fragment used in the methods described herein induces at least 10% less, at least 20% less, at least 30% less, at least 40% less, at least 50% less, at least 60% less, at least 70% less, at least 80% less, at least 90% less, at least 95% less, or at least 99% less degranulation of FcgRI-expressing human mast cells (e.g., as determined, for example, by % release of beta-hexosaminidase from human mast cells in culture (e.g., in presence of IFN gamma)) compared to an appropriate control antibody or antigen binding fragment. In particular embodiments, release of beta-hexosaminidase from human mast cells in culture in presence of IFN gamma is reduced by more than 50% with an anti-KIT antibody or antigen binding fragment used in the methods described herein compared to an appropriate control antibody or antigen-binding fragment. In particular embodiments, release of beta-hexosaminidase from human mast cells in culture in presence of IFN gamma is reduced by more than 60%, more than 70%, or more than 80% with an anti-KIT antibody or antigen binding fragment used in the methods described herein compared to an appropriate control antibody or antigen-binding fragment. When an anti-KIT antibody or antigen binding fragment used in the methods described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain), in preferred embodiments the appropriate control antibody or antigen binding fragment is an antibody or antigen binding fragment having the same VH and VL but with a wild-type (unmodified) Fc region or domain of the same isotype. In particular embodiments, an anti-KIT antibody or antigen binding fragment used in the methods described herein comprises a modified (e.g., mutated) human IgG1 Fc region or domain and induces at least 10% less, at least 20% less, at least 30% less, at least 40% less, at least 50% less, at least 60% less, at least 70% less, at least 80% less, at least 90% less, at least 95% less, or at least 99% less degranulation of FcgRI-expressing human mast cells (e.g., as determined, for example, by % release of beta-hexosaminidase from human mast cells in culture (e.g., in presence of IFN gamma)) compared to a corresponding antibody or antigen binding fragment having the same VH and VL but with a wild-type (unmodified) human IgG1 Fc region or domain. In particular embodiments, release of beta-hexosaminidase from human mast cells in culture in presence of IFN gamma is reduced by more than 50% with an anti-KIT antibody or antigen binding fragment used in the methods described herein that comprises a modified (e.g., mutated) human IgG1 Fc region or domain, compared to a corresponding antibody or antigen binding fragment having the same VH and VL but with a wild-type (unmodified) human IgG1 Fc region or domain. In particular embodiments, release of beta-hexosaminidase from human mast cells in culture in presence of IFN gamma is reduced by more than 60%, more than 70%, or more than 80% with an anti-KIT antibody or antigen binding fragment used in the methods described herein that comprises a modified (e.g., mutated) human IgG1 Fc region or domain, compared to a corresponding antibody or antigen binding fragment having the same VH and VL but with a wild-type (unmodified) human IgG1 Fc region or domain.

In specific embodiments, an anti-KIT antibody or antigen binding fragment used in the methods described herein does not show significant Fc receptor-dependent KIT agonistic activity (e.g., as determined, for example, by KIT phosphorylation). In specific embodiments, an anti-KIT antibody or antigen binding fragment used in the methods described herein does not show detectable Fc receptor-dependent KIT agonistic activity (e.g., as determined, for example, by KIT phosphorylation). In particular embodiments, an anti-KIT antibody or antigen binding fragment used in the methods described herein induces at least 10% less, at least 20% less, at least 30% less, at least 40% less, at least 50% less, at least 60% less, at least 70% less, at least 80% less, at least 90% less, at least 95% less, or at least 99% less Fc receptor-dependent KIT activity (e.g., as determined, for example, by KIT phosphorylation) compared to an appropriate control antibody or antigen binding fragment. In particular embodiments, Fc receptor-dependent KIT agonist activity (as determined by KIT phosphorylation with Fc receptors crosslinked) is reduced by more than 50% with an anti-KIT antibody or antigen binding fragment used in the methods described herein compared to an appropriate control antibody or antigen binding fragment. In particular embodiments, Fc receptor-dependent KIT agonist activity (as determined by KIT phosphorylation with Fc receptors crosslinked) is reduced by more than 60%, more than 70%, or more than 80% with an anti-KIT antibody or antigen binding fragment used in the methods described herein compared to an appropriate control antibody or antigen binding fragment. When an anti-KIT antibody or antigen binding fragment used in the methods described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain), in preferred embodiments the appropriate control antibody or antigen binding fragment is an antibody or antigen binding fragment having the same VH and VL but with a wild-type (unmodified) Fc region or domain of the same isotype. In particular embodiments, an anti-KIT antibody or antigen binding fragment used in the methods described herein comprises a modified (e.g., mutated) human IgG1 Fc region or domain and induces at least 10% less, at least 20% less, at least 30% less, at least 40% less, at least 50% less, at least 60% less, at least 70% less, at least 80% less, at least 90% less, at least 95% less, or at least 99% less Fc receptor-dependent KIT activity (e.g., as determined, for example, by KIT phosphorylation) compared to a corresponding antibody or antigen binding fragment having the same VH and VL but with a wild-type (unmodified) human IgG1 Fc region or domain. In specific embodiments, an anti-KIT antibody or antigen binding fragment used in the methods described herein does not show significant or detectable Fc receptor-dependent KIT agonistic activity as described herein even when cross-linked on THP-1 cells. In particular embodiments, Fc receptor-dependent KIT agonist activity (as determined by KIT phosphorylation with Fc receptors crosslinked) is reduced by more than 50% with an anti-KIT antibody or antigen binding fragment used in the methods described herein that comprises a modified (e.g., mutated) human IgG1 Fc region or domain, compared to a corresponding antibody or antigen binding fragment having the same VH and VL but with a wild-type (unmodified) human IgG1 Fc region or domain. In particular embodiments, Fc receptor-dependent KIT agonist activity (as determined by KIT phosphorylation with Fc receptors crosslinked) is reduced by more than 60%, more than 70%, or more than 80% with an anti-KIT antibody or antigen binding fragment used in the methods described herein that comprises a modified (e.g., mutated) human IgG1 Fc region or domain, compared to a corresponding antibody or antigen binding fragment having the same VH and VL but with a wild-type (unmodified) human IgG1 Fc region or domain.

In various embodiments, an anti-KIT antibody or antigen binding fragment used in the methods described herein (1) reduces disease activity in a chronic prurigo patient, (2) reduces skin mast cell number in a chronic prurigo patient, (3) reduces tryptase level in a chronic prurigo patient, and/or (4) maintains hematology parameters (such as hemoglobin (HgB) level, white blood cell (WBC) count, platelet count, and/or absolute neutrophil count (ANC)) in a patient such as a chronic prurigo patient within the normal ranges.

In certain embodiments, an anti-KIT antibody or antigen binding fragment used in the methods described herein can significantly reduce skin mast cell number in a chronic prurigo patient, relative to the number before treatment. In specific embodiments, an anti-KIT antibody or antigen binding fragment used in the methods described herein can reduce skin mast cell number in a chronic prurigo patient by at least 20%, at least 40%, at least 60%, or at least 80% (e.g., in a week, in 2 weeks, in 4 weeks, in 6 weeks, in 8 weeks, in 10 weeks, or in 12 weeks after treatment with the anti-KIT antibody or antigen binding fragment), relative to the number before treatment. In specific embodiments, the effect of the anti-KIT antibody or antigen binding fragment used in the methods described herein is sustained for at least 2 weeks, at least 4 weeks, at least 6 weeks, at least 8 weeks, at least 10 weeks, or at least 12 weeks.

In certain embodiments, an anti-KIT antibody or antigen binding fragment used in the methods described herein can significantly reduce serum tryptase in a chronic prurigo patient, relative to the level before treatment. In specific embodiments, an anti-KIT antibody or antigen binding fragment used in the methods described herein can reduce serum tryptase in a chronic prurigo patient by at least 50%, at least 70%, or at least 90% (e.g., in a week, in 2 weeks, in 4 weeks, in 6 weeks, in 8 weeks, in 10 weeks, or in 12 weeks after treatment with the anti-KIT antibody or antigen binding fragment), relative to the level before treatment. In specific embodiments, the effect of the anti-KIT antibody or antigen binding fragment used in the methods described herein is sustained for at least 2 weeks, at least 4 weeks, at least 6 weeks, at least 8 weeks, at least 10 weeks, or at least 12 weeks.

In certain embodiments, an anti-KIT antibody or antigen binding fragment used in the methods described herein maintains hematology parameters (such as hemoglobin (HgB) level, white blood cell (WBC) count, platelet count, and/or absolute neutrophil count (ANC)) in a patient within the normal ranges. In certain embodiments, an anti-KIT antibody or antigen binding fragment used in the methods described herein maintains hematology parameters (such as hemoglobin (HgB) level, white blood cell (WBC) count, platelet count, and/or absolute neutrophil count (ANC)) in a chronic prurigo patient within the normal ranges. In specific embodiments, the hematology parameters are maintained for at least 2 weeks, at least 4 weeks, at least 6 weeks, at least 8 weeks, at least 10 weeks, or at least 12 weeks.

In various embodiments, an anti-KIT antibody or antigen binding fragment used in the methods described herein has one or more of the properties described herein.

In specific embodiments, an antibody described herein comprises a modified (e.g., mutated) Fc region or domain, wherein the Fc region or domain comprises at least one (e.g., one, two, three, four, five or six) amino acid modifications (e.g. substitution, deletion or addition) or at least one (e.g., one, two, three, four, five or six) non-naturally occurring amino acid residues.

In a specific embodiment, an antibody described herein comprises a modified (e.g., mutated) Fc region or domain, wherein the Fc region or domain is an Fc region or domain of human IgG1 and comprises at least one (e.g., one, two, three, four, five or six) amino acid modifications (e.g. substitution, deletion or addition) or at least one (e.g., one, two, three, four, five or six) non-naturally occurring amino acid residues selected from the group consisting of 234A, 234D, 234E, 234N, 234Q, 234T, 23411, 234Y, 2341, 234V, 234F, 235A, 235D, 235R, 235W, 235P, 235S, 235N, 235Q, 235T, 23511, 235Y, 2351, 235V, 235F, 236E, 239D, 239E, 239N, 239Q, 239F, 239T, 23911, 239Y, 2401, 240A, 240T, 240M, 241W, 241L, 241Y, 241E, 241R. 243W, 243L 243Y, 243R, 243Q, 244H, 245A, 247V, 247G, 252Y, 254T, 256E, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 26911, 269Y, 269F, 269R, 296E, 296Q, 296D, 296N, 296S, 296T, 296L, 2961, 29611, 269G, 297S, 297D, 297E, 29811, 2981, 298T, 298F, 2991, 299L, 299A, 299S, 299V, 29911, 299F, 299E, 313F, 322Q, 325Q, 325L, 325I, 325D, 325E, 325A, 325T, 325V, 32511, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 332D, 332S, 332W, 332F, 332E, 332N, 332Q, 332T, 33211, 332Y, and 332A as numbered by the EU index as set forth in Kabat. Optionally, the Fc region or domain may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 04/016750; WO 04/029207; WO 04/035752 and WO 05/040217). In a specific embodiment, an antibody described herein comprises a modified (e.g., mutated) Fc region or domain, wherein the Fc region or domain is an Fc region or domain of human IgG2 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified (e.g., mutated) Fc region or domain, wherein the Fc region or domain is an Fc region or domain of human IgG3 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified (e.g., mutated) Fc region or domain, wherein the Fc region or domain is an Fc region or domain of human IgG4 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art.

In a specific embodiment, an antibody described herein comprises a modified (e.g., mutated) Fc region or domain, wherein the Fc region or domain is an Fc region or domain of human IgG1 and comprises at least one (e.g., one, two, three, four, five or six) amino acid modifications (e.g. substitution, deletion or addition) or at least one non-naturally occurring amino acid residue (e.g., one, two, three, four, five or six) selected from the group consisting of 234A, 234D, 234E, 234N, 234Q, 234T, 23411, 234Y, 2341, 234V, 234F, 235A, 235D, 235R, 235W, 235P, 235S, 235N, 235Q, 235T, 23511, 235Y, 2351, 235V, 235F, 236E, 239D, 239E, 239N, 239Q, 239F, 239T, 23911, 239Y, 2401, 240A, 240T, 240M, 241W, 241L, 241Y, 241E, 241R. 243W, 243L 243Y, 243R, 243Q, 244H, 245A, 247V, 247G, 252Y, 254T, 256E, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 26511, 265T, 2661, 266A, 266T, 266M, 267Q, 267L, 26911, 269Y, 269F, 269R, 296E, 296Q, 296D, 296N, 296S, 296T, 296L, 2961, 29611, 269G, 297S, 297D, 297E, 29811, 2981, 298T, 298F, 2991, 299L, 299A, 299S, 299V, 29911, 299F, 299E, 313F, 322Q, 325Q, 325L, 325I, 325D, 325E, 325A, 325T, 325V, 32511, 327G, 327W, 327N, 327L, 328S, 328M, 328D, 328E, 328N, 328Q, 328F, 328I, 328V, 328T, 32811, 328A, 329F, 32911, 329Q, 330K, 330G, 330T, 330C, 330L, 330Y, 330V, 3301, 330F, 330R, 330H, 332D, 332S, 332W, 332F, 332E, 332N, 332Q, 332T, 33211, 332Y, and 332A as numbered by the EU index as set forth in Kabat. Optionally, the Fc region or domain may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 04/016750; WO 04/029207; WO 04/035752 and WO 05/040217). In a specific embodiment, an antibody described herein comprises a modified (e.g., mutated) Fc region or domain, wherein the Fc region or domain is an Fc region or domain of human IgG2 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified (e.g., mutated) Fc region or domain, wherein the Fc region or domain is an Fc region or domain of human IgG3 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified (e.g., mutated) Fc region or domain, wherein the Fc region or domain is an Fc region or domain of human IgG4 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art.

In a certain aspect, provided herein is an antibody comprising an Fc region or domain, wherein the Fc region or domain is an Fc region or domain of human IgG1 and comprises at least a non-naturally occurring amino acid at one or more positions selected from the group consisting of 239, 330 and 332, as numbered by the EU index as set forth in Kabat. In a specific embodiment, provided herein is an antibody comprising an Fc region or domain, wherein the Fc region or domain is an Fc region or domain of human IgG1 and comprises at least one non-naturally occurring amino acid selected from the group consisting of 239D, 330L and 332E, as numbered by the EU index as set forth in Kabat. Optionally, the Fc region or domain may further comprise additional non-naturally occurring amino acid at one or more positions selected from the group consisting of 252, 254, and 256, as numbered by the EU index as set forth in Kabat. In a specific embodiment, provided herein is an antibody comprising an Fc region or domain, wherein the Fc region or domain is an Fc region or domain of human IgG1 and comprises at least one non-naturally occurring amino acid selected from the group consisting of 239D, 330L and 332E, as numbered by the EU index as set forth in Kabat and at least one non-naturally occurring amino acid at one or more positions are selected from the group consisting of 252Y, 254T and 256E, as numbered by the EU index as set forth in Kabat. In a specific embodiment, provided herein is an antibody comprising an Fc region or domain, wherein the Fc region or domain is an Fc region or domain of human IgG2, IgG3, or IgG4, and comprises at least one non-naturally occurring amino acid residue that is an equivalent(s) to the amino acid residue(s) described herein for a human IgG1 Fc region or domain, as can be determined by one of skill in the art. In a specific embodiment, provided herein is an antibody comprising an Fc region or domain, wherein the Fc region or domain is an Fc region or domain of human IgG2, IgG3, or IgG4, and comprises at least one non-naturally occurring amino acid residue at one or more positions that are equivalent(s) to the positions described herein for a human IgG1 Fc region or domain, as can be determined by one of skill in the art. In one embodiment, an Fc region or domain comprising such sequence exhibits one or more Fc activity, for example, binding affinity to an Fc receptor or effector function, such as ADCC or CDC. In a specific embodiment, an Fc region or domain comprising such sequence exhibits reduced Fc activity, for example, reduced binding affinity to an Fc receptor or reduced effector function, such as ADCC or CDC. In a particular embodiment, an Fc region or domain comprising such sequence exhibits enhanced FcRn activity, for example, enhanced half-life.

Additional non-limiting examples of Fc region or domain modifications are provided in Ghetie et al., 1997, Nat Biotech. 15:637-40; Duncan et al., 1988, Nature 332:563-564; Lund et at, 1991, J. Immunol 147:2657-2662; Lund et al., 1992, Mol Immunol 29:53-59; Alegre et al., 1994, Transplantation 57: 1537-1543; Hutchins et al., 1995, Proc Natl. Acad Sci USA 92: 11980-11984; Jefferis et al., 1995, Immunol Lett. 44: 111-117; Lund et al., 1995, Faseb J 9: 115-119; Jefferis et al., 1996, Immunol Lett 54: 101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164: 1925-1933; Xu et al., 2000, Cell Immunol 200: 16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; 8,163,882; 7,355,008; 7,960,512; 8,039,592; 8,039,359; 8,101,720; 7,214,775; 7,682,610; 7,741,442; U.S. Patent Publication Nos. 2004/0002587 and PCT Publications WO 94/29351; WO 99/58572; WO 00/42072; WO 04/029207; WO 04/099249; WO 04/063351.

In specific embodiments, the antibody described herein comprises a modified (e.g., mutated) human IgG1 Fc region or domain, which comprises non-naturally occurring amino acids 234A, 235Q and 322Q as numbered by the EU index as set forth in Kabat. In a particular embodiment, the modified (e.g., mutated) human IgG1 Fc region or domain further comprises non-naturally occurring amino acids 252Y, 254T and 256E as numbered by the EU index as set forth in Kabat.

In certain embodiments, the antibody described herein comprises a modified (e.g., mutated) human IgG2 Fc region or domain, which comprises non-naturally occurring amino acids that are equivalents to 234A, 235Q and 322Q as numbered by the EU index as set forth in Kabat for human IgG1 Fc region or domain, as can be determined by one of skill in the art. In certain embodiments, the antibody described herein comprises a modified (e.g., mutated) human IgG2 Fc region or domain, which comprises non-naturally occurring amino acids that are equivalents to 234A, 235Q, 322Q, 252Y, 254T and 256E as numbered by the EU index as set forth in Kabat for human IgG1 Fc region or domain, as can be determined by one of skill in the art.

In certain embodiments, the antibody described herein comprises a modified (e.g., mutated) human IgG3 Fc region or domain, which comprises non-naturally occurring amino acids that are equivalents to 234A, 235Q and 322Q as numbered by the EU index as set forth in Kabat for human IgG1 Fc region or domain, as can be determined by one of skill in the art. In certain embodiments, the antibody described herein comprises a modified (e.g., mutated) human IgG3 Fc region or domain, which comprises non-naturally occurring amino acids that are equivalents to 234A, 235Q, 322Q, 252Y, 254T and 256E as numbered by the EU index as set forth in Kabat for human IgG1 Fc region or domain, as can be determined by one of skill in the art.

In certain embodiments, the antibody described herein comprises a modified (e.g., mutated) human IgG4 Fc region or domain, which comprises non-naturally occurring amino acids that are equivalents to 234A, 235Q and 322Q as numbered by the EU index as set forth in Kabat for human IgG1 Fc region or domain, as can be determined by one of skill in the art. In certain embodiments, the antibody described herein comprises a modified (e.g., mutated) human IgG4 Fc region or domain, which comprises non-naturally occurring amino acids that are equivalents to 234A, 235Q, 322Q, 252Y, 254T and 256E as numbered by the EU index as set forth in Kabat for human IgG1 Fc region or domain, as can be determined by one of skill in the art.

In a specific embodiment, the antibody described herein comprises the VL and VH CDR sequences set forth in Table 1 and a modified (e.g., mutated) human IgG1 Fc region or domain, wherein the modified (e.g., mutated) human IgG1 Fc region or domain comprises non-naturally occurring amino acids 234A, 235Q, and 322Q as numbered by the EU index as set forth in Kabat.

In a preferred embodiment, the antibody described herein comprises the VL and VH CDR sequences set forth in Table 1 and a modified (e.g., mutated) human IgG1 Fc region or domain, wherein the modified (e.g., mutated) human IgG1 Fc region or domain comprises non-naturally occurring amino acids 234A, 235Q, 322Q, 252Y, 254T and 256E as numbered by the EU index as set forth in Kabat.

Thus, in one aspect, provided herein is an antibody, or an antigen-binding fragment thereof, which immunospecifically binds to human KIT, comprising:
(i) a VL comprising a VL CDR1, VL CDR2, and VL CDR3 having the amino acid sequences of SEQ ID NO: 2, SEQ ID NO: 3, and SEQ ID NO: 4, respectively; (ii) a VH comprising VH CDR1, VH CDR2, and VH CDR3 having the amino acid sequences of SEQ ID NO: 5, SEQ ID NO: 6, and SEQ ID NO: 7, respectively; and (iii) a modified (e.g., mutated) human IgG1 Fc region or domain comprising non-naturally occurring amino acids 234A, 235Q, and 322Q as numbered by the EU index as set forth in Kabat. In a specific embodiment, the antibody or antigen-binding fragment thereof provided herein is an antibody.

Thus, in a further aspect, provided herein is an antibody, or an antigen-binding fragment thereof, which immunospecifically binds to human KIT, comprising:
(i) a VL comprising a VL CDR1, VL CDR2, and VL CDR3 having the amino acid sequences of SEQ ID NO: 2, SEQ ID NO: 3, and SEQ ID NO: 4, respectively; (ii) a VH comprising VH CDR1, VH CDR2, and VH CDR3 having the amino acid sequences of SEQ ID NO: 5, SEQ ID NO: 6, and SEQ ID NO: 7, respectively; and (iii) a modified (e.g., mutated) human IgG1 Fc region or domain comprising non-naturally occurring amino acids 234A, 235Q, 322Q, 252Y, 254T and 256E as numbered by the EU index as set forth in Kabat. In a specific embodiment, the antibody or antigen-binding fragment thereof provided herein is an antibody.

In a further aspect, provided herein is an antibody, or an antigen-binding fragment thereof, which immunospecifically binds to human KIT, comprising: (i) a VL comprising the amino acid sequence: DIVMTQSPSX_{K1}LSASVGDRVTITCKASQNVRTNVAWYQQKPGKAPKX_{K2}LIYS ASYRYSGVPDRFX_{K3}GSGSGTDFTLTISSLQX_{K4}EDFAX_{K5}YX_{K6}CQQYNSYPRTFGGGTKV EIK (SEQ ID NO: 17), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and (ii) a VH comprising the amino acid sequence: QVQLVQSGAEX_{H1}KKPGASVKX_{H2}SCKASGYTFTDYYINWVX_{H3}QAPGKGLEWIARIYPG SGNTYYNEKFKGRX_{H4}TX_{H5}TAX_{H6}KSTSTAYMX_{H7}LSSLRSEDX_{H8}AVYFCARGVYYFDY WGQGTTVTVSS (SEQ ID NO: 18) wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain; and (iii) a modified (e.g., mutated) human IgG1 Fc region or domain comprising non-naturally occurring amino acids 234A, 235Q, 322Q and preferably also 252Y, 254T and 256E as numbered by the EU index as set forth in Kabat. In a specific embodiment, the antibody or antigen-binding fragment thereof provided herein is an antibody.

In a further aspect, provided herein is an antibody, or an antigen-binding fragment thereof, which immunospecifically binds to human KIT, comprising: i) a VL which comprises the amino acid sequence of SEQ ID NO: 13, 14, 15, or 16, and ii) a VH comprising the amino acid sequence of SEQ ID NO: 8, 9, 10, 11, or 12; and (iii) a modified (e.g., mutated) human IgG1 Fc region or domain comprising non-naturally occurring amino acids 234A, 235Q, 322Q and preferably also 252Y, 254T and 256E as numbered by the EU index as set forth in Kabat. In a specific embodiment, the antibody or antigen-binding fragment thereof provided herein is an antibody.

In a further aspect, provided herein is an antibody, or an antigen-binding fragment thereof, which immunospecifically binds to human KIT, comprising: i) a VL which comprises the amino acid sequence of SEQ ID NO: 14 and ii) a VH which comprises the amino acid sequence of SEQ ID NO: 10; and (iii) a modified (e.g., mutated) human IgG1 Fc region or domain comprising non-naturally occurring amino acids 234A, 235Q, 322Q and preferably also 252Y, 254T and 256E as numbered by the EU index as set forth in Kabat. In a specific embodiment, the antibody or antigen-binding fragment thereof provided herein is an antibody.

In specific embodiments, the antibody provided herein comprises a heavy chain comprising the following amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG.

In specific embodiments, the antibody provided herein comprises a light chain comprising the following amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In specific embodiments, the antibody provided herein comprises a heavy chain comprising the following amino acid sequence: QVQLVQSGAEVKKPGASVKLSCKASGYTFTDYYINWVRQAPGKGLEWIARIYPGSGNT YYNEKFKGRATLTADKSTSTAYMQLSSLRSEDTAVYFCARGVYYFDYWGQGTTVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAQG GPSVFLFPPKPKDTLYITREPEVTCVVVDVSHEDPEVKFNWYVDGVEVHNAKTKPREEQ YNSTYRVVSVLTVLHQDWLNGKEYKCQVSNKALPAPIEKTISKAKGQPREPQVYTLPPS RDELTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 21); and a light chain comprising the following amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In a specific embodiment, provided herein is an antibody comprising: (i) a heavy chain comprising the amino acid sequence:

(SEQ ID NO: 19)
MEWSWVFLFFLSVTTGVHSQVQLVQSGAEVKKPGASVKLSCKASGYTFTDYYINWVRQAP
GKGLEWIARIYPGSGNTYYNEKFKGRATLTADKSTSTAYMQLSSLRSEDTAVYFCARGVYYFDY
WGQGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTC
PPCPAPE [symbol] GGPSVFLFPPKPKDTL [symbol] I [symbol] R [symbol] PEVTCVVVDVSHEDPEVKFNWYVDGVEVH
NAKTKPREEQYNSTYRVVSVLTVLHQDWLNGKEYKC [symbol] VSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG,

wherein the leader sequence is shown in bold italic type, the variable region (VH) is shown in italic type and the constant region is shown underlined. In addition the mutations in the constant region (compared to wild type human IgG1) are shown double underlined; and (ii) a light chain comprising the amino acid sequence:

(SEQ ID NO: 20)
MSVPTQVLGLLLLWLTDARCDIVMTQSPSSLSASVGDRVTITCKASQNV
RTNVAWYQQKPGKAPKALIYSASYRYSGVPDRFTGSGSGTDFTLTISSL
QPEDFADYFCQQYNSYPRTFGGGTKVEIKRTVAAPSVFIFPPSDEQLKS
GTASVVCLLNNFYPREAKVQWKVDNALQSGNSQESVTEQDSKDSTYSLS
STLTLSKADYEKHKVYACEVTHQGLSSPVTKSFNRGEC,

wherein the leader sequence is shown in bold italic type, the variable region (VL) is shown in italic type and the constant region is shown underlined.

In specific embodiments, an anti-KIT antibody described herein does not bind to (e.g., has no detectable binding to) any human Fc-gamma receptor (FcγR receptor). In a specific embodiment, an anti-KIT antibody described herein does not bind to (e.g., has no detectable binding to) human FcγRI. In a specific embodiment, an anti-KIT antibody described herein does not bind to (e.g., has no detectable binding to) human FcγRIIa. In a specific embodiment, an anti-KIT antibody described herein does not bind to (e.g., has no detectable binding to) human FcγRIIb. In a specific embodiment, an anti-KIT antibody described herein does not bind to (e.g., has no detectable binding to) human FcγRIIIa. In a specific embodiment, an anti-KIT antibody described herein does not bind to (e.g., has no detectable binding to) human FcγRIIIb.

In specific embodiments, an anti-KIT antibody described herein comprises a modified (e.g., mutated) human IgG constant region (e.g., a modified (e.g., mutated) human IgG1, IgG2, IgG3, or IgG4 constant region) and has an enhanced binding (e.g., an at least 2-fold, 5-fold, 10-fold, 50-fold, 100-fold, 500-fold, 1000-fold, 5000-fold, or 10000-fold higher binding affinity) to human neonatal Fc Receptor (FcRn) relative to a corresponding antibody with the same variable region sequences but an unmodified (wild type) human IgG constant region. In a specific embodiment, an anti-KIT antibody described herein binds to FcRn at pH 6.0 with a KD of less than 20 nM. In a specific embodiment, an anti-KIT antibody described herein binds to FcRn at pH 6.0 with a KD of less than 2 nM. In a specific embodiment, an anti-KIT antibody described herein binds to FcRn at pH 6.0 with a KD of less than 1 nM. In a specific embodiment, an anti-KIT antibody described herein binds to FcRn at pH 6.0 with a KD of less than 500 nM. In a specific embodiment, an anti-KIT antibody described herein binds to FcRn at pH 6.0 with a KD of less than 400 pM. In a specific embodiment, an anti-KIT antibody described herein binds to FcRn at pH 7.2 with a KD of less than 200 nM. In a specific embodiment, an anti-KIT antibody described herein binds to FcRn at pH 7.2 with a KD of less than 150 nM. In a specific embodiment, an anti-KIT antibody described herein binds to FcRn at pH 7.2 with a KD of less than 100 nM. In a specific embodiment, an anti-KIT antibody described herein binds to FcRn at pH 7.2 with a KD of less than 80 nM.

In specific embodiments, an anti-KIT antibody described herein comprises a modified (e.g., mutated) human IgG constant region (e.g., a modified (e.g., mutated) human IgG1, IgG2, IgG3, or IgG4 constant region) and exhibits no antibody-dependent cellular cytotoxicity (ADCC). In specific embodiments, an anti-KIT antibody described herein comprises a modified (e.g., mutated) human IgG constant region (e.g., a modified (e.g., mutated) human IgG1, IgG2, IgG3, or IgG4 constant region) and exhibits reduced (e.g., at least 10% less, at least 20% less, at least 30% less, at least 40% less, at least 50% less, at least 60% less, at least 70% less, at least 80% less, at least 90% less, at least 95% less, or at least 99% less) ADCC, relative to a corresponding antibody with the same variable region sequences but an unmodified (wild type) human IgG constant region.

In specific embodiments, an anti-KIT antibody described herein comprises a modified (e.g., mutated) human IgG constant region (e.g., a modified (e.g., mutated) human IgG1, IgG2, IgG3, or IgG4 constant region) and exhibits reduced (e.g., at least 10% less, at least 20% less, at least 30% less, at least 40% less, at least 50% less, at least 60% less, at least 70% less, at least 80% less, at least 90% less, at least 95% less, or at least 99% less) production of cytokines (e.g., IFN-γ, IL-1β, IL-2, IL-6, 1L-8, 1L-10, and/or TNF-α), relative to a corresponding antibody with the same variable region sequences but an unmodified (wild type) human IgG constant region.

In certain aspects, anti-KIT antibodies or antigen binding fragments thereof for use in methods for preventing, treating or managing chronic prurigo, have been described or can be readily obtained using methods known in the art, for example, see Section 5.2 below.

In a particular aspect, an anti-KIT antibody or an antigen binding fragment thereof for use in methods provided herein specifically binds to a D4 domain of human KIT and a D5 region of KIT, e.g., human KIT. In another specific embodiment, an anti-KIT antibody or an antigen binding fragment thereof for use in methods provided herein specifically binds to a D5 domain of KIT, e.g., human KIT, with lower affinity than to a D4 domain of KIT, e.g., human KIT. In a particular embodiment, an anti-KIT antibody or an antigen binding fragment thereof for use in methods provided herein specifically binds to a D4 domain of KIT, e.g., human KIT, with higher affinity than to a D5 domain of KIT, e.g., human KIT; for example, the higher affinity is at least 1 fold, 2 fold, 3 fold, 4 fold, 5 fold, 10 fold, 20 fold, 50 fold, 100 fold, 500 fold, or 1000 fold as determined by methods known in the art, e.g., ELISA or Biacore assays.

In a particular embodiment, an anti-KIT antibody or an antigen binding fragment thereof for use in methods provided herein specifically binds to a D4 or D4/D5 region of KIT, e.g., human KIT, and has at least 1 fold, 2 fold, 3 fold, 4 fold, 5 fold, or 10 fold higher affinity for a KIT antigen consisting essentially of a D4 domain only than a KIT antigen consisting essentially of a D5 domain only.

In a particular embodiment, an anti-KIT antibody or an antigen binding fragment thereof for use in methods provided herein specifically binds to a KIT polypeptide (e.g., the D4 region of human KIT) with an EC₅₀ (half maximal effective concentration) value of about 50 nM, 10 nM, 500 pM, 300 pM, 200 pM, 100 pM or 50 pM or less as determined by an assay described in the art, such as ELISA.

In a particular embodiment, an anti-KIT antibody or an antigen binding fragment thereof for use in methods provided herein specifically binds to a KIT polypeptide (e.g., the D4 region of human KIT) with an EC₅₀ value of about 200 pM or 150 pM or less as determined by an assay described in the art, such as ELISA or FACs with CHO-WT-KIT cells (CHO cells engineered to recombinantly express wild-type human KIT).

In a particular embodiment, an anti-KIT antibody or an antigen binding fragment thereof for use in methods provided herein is capable of blocking KIT phosphorylation with IC₅₀ (50% inhibition concentration) value of about 600 pM or less.

In a particular embodiment, an anti-KIT antibody or an antigen binding fragment thereof for use in methods provided herein is capable of inducing or enhancing KIT receptor internalization, e.g., by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, relative to internalization in the presence of an unrelated antibody (e.g., an antibody that does not immunospecifically bind to KIT). In a particular embodiment, an anti-KIT antibody or an antigen binding fragment thereof for use in methods provided herein is capable of inducing or enhancing KIT receptor internalization, e.g., by at least about 25% or 35%, optionally to about 75%, as assessed by methods described herein or known to one of skill in the art, relative to internalization in the presence of an unrelated antibody (e.g., an antibody that does not immunospecifically bind to KIT). In a particular embodiment, an anti-KIT antibody or an antigen binding fragment thereof for use in methods provided herein is capable of inducing or enhancing KIT receptor internalization, e.g., by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, relative to internalization in the presence of an unrelated antibody (e.g., an antibody that does not immunospecifically bind to KIT). Techniques for the quantitation or visualization of cell surface receptors are well known in the art and include a variety of fluorescent and radioactive techniques. For example, one method involves incubating the cells with a radiolabeled anti-receptor antibody. Alternatively, the natural ligand of the receptor can be conjugated to a fluorescent molecule or radioactive-label and incubated with the cells. Additional receptor internalization assays are well known in the art and are described in, for example, Jimenez et al., Biochemical Pharmacology, 1999, 57:1125-1131; Bernhagen et al., Nature Medicine, 2007, 13:587-596; and Conway et al., J. Cell Physiol., 2001, 189:341-55.

In a particular embodiment, an anti-KIT antibody or an antigen binding fragment thereof for use in methods provided herein is capable of inducing or enhancing KIT receptor turnover, e.g., by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., pulse-chase assay), relative to turnover in the presence of an unrelated antibody (e.g., an antibody that does not immunospecifically bind to KIT). In a particular embodiment, an anti-KIT antibody or an antigen binding fragment thereof for use in methods provided herein is capable of inducing or enhancing KIT receptor turnover, by at least about 25% or 35%, optionally to about 75%, as assessed by methods described herein or known to one of skill in the art (e.g., pulse-chase assay), relative to turnover in the presence of an unrelated antibody (e.g., an antibody that does not immunospecifically bind to KIT). In a particular embodiment, an anti-KIT antibody or an antigen binding fragment thereof for use in methods provided herein is capable of inducing or enhancing KIT receptor turnover, by at least about 1 fold, 1.2 fold, 1.3 fold, 1.4 fold, 1.5 fold, 2 fold, 2.5 fold, 3 fold, 3.5 fold, 4 fold, 4.5 fold, fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., pulse-chase assay), relative to turnover in the presence of an unrelated antibody (e.g., an antibody that does not immunospecifically bind to KIT). Methods for the determining receptor turnover are well known in the art. For example, cells expressing KIT can be pulse-labeled using ³⁵S-EXPRESS Protein Labeling mix (NEG772, NEN Life Science Products), washed and chased with unlabeled medium for a period of time before protein lysates from the labeled cells are immunoprecipitated using an anti-KIT antibody and resolved by SDS-PAGE and visualized (e.g., exposed to a Phospholmager screen (Molecular Dynamics), scanned using the Typhoon8600 scanner (Amersham), and analyzed using ImageQuant software (Molecular Dynamics)) (see, e.g., Chan et al., Development, 2004, 131:5551-5560).

In a particular embodiment, an anti-KIT antibody or an antigen binding fragment thereof for use in methods provided herein is capable of inducing or enhancing KIT receptor degradation, by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., pulse-chase assays), relative to degradation in the presence of an unrelated antibody (e.g., an antibody that does not immunospecifically bind to KIT). In a particular embodiment, an anti-KIT antibody or an antigen binding fragment thereof for use in methods provided herein is capable of inducing or enhancing KIT receptor degradation, by at least about 25% or 35%, optionally to about 75%, as assessed by methods described herein or known to one of skill in the art (e.g., pulse-chase assays), relative to degradation in the presence of an unrelated antibody (e.g., an antibody that does not immunospecifically bind to KIT). In a particular embodiment, an anti-KIT antibody or an antigen binding fragment thereof for use in methods provided herein is capable of inducing or enhancing KIT receptor degradation, by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., pulse-chase assays), relative to degradation in the presence of an unrelated antibody (e.g., an antibody that does not immunospecifically bind to KIT). Techniques for quantitating or monitoring ubiquitination and/or degradation (e.g., kinetics or rate of degradation) of cell surface receptors are well known in the art and involve a variety of fluorescent and radioactive techniques (see, e.g., International Patent Application Publication No. WO 2008/153926 A2). For example, pulse chase experiments or experiments using radiolabeled ligands such as ¹²⁵I-SCF can be carried out to quantitatively measure degradation of KIT.

In particular embodiments, an anti-KIT antibody or an antigen binding fragment thereof for use in methods provided herein does not bind the extracellular ligand binding site of KIT, e.g., the SCF binding site of KIT. In particular embodiments, an anti-KIT antibody or an antigen binding fragment thereof for use in methods provided herein does not inhibit ligand binding to KIT, e.g., does not inhibit KIT ligand (e.g., SCF) binding to KIT, as determined by a method described in the art, for example, ELISA. In certain embodiments, an anti-KIT antibody or an antigen binding fragment thereof for use in methods provided herein does not fully inhibit, or partially inhibits, ligand binding to KIT, e.g., does not fully inhibit, or partially inhibits, KIT ligand (e.g., SCF) binding to KIT, as determined by a method described in the art, for example, ELISA or FACS (fluorescence-activated cell sorting).

In specific aspects, anti-KIT antibodies (e.g., human or humanized antibodies) for use in the methods provided herein are inhibitory antibodies, that is, antibodies that inhibit (e.g., partially inhibit) KIT activity, i.e., one or more KIT activities. In a specific embodiment, partial inhibition of a KIT activity results in, for example, about 25% to about 65% or 75% inhibition. In a specific embodiment, partial inhibition of a KIT activity results in, for example, about 35% to about 85% or 95% inhibition. Non-limiting examples of KIT activities include KIT dimerization, KIT phosphorylation (e.g., tyrosine phosphorylation), signaling downstream of KIT (e.g. Stat, AKT, MAPK, or Ras signaling), induction or enhancement of gene transcription (e.g., c-Myc), induction or enhancement of cell proliferation or cell survival. In a particular embodiment, an antibody described herein inhibits KIT phosphorylation (e.g., ligand-induced phosphorylation).

In a specific embodiment, an anti-KIT antibody or an antigen binding fragment thereof for use in methods provided herein inhibits KIT tyrosine phosphorylation in the KIT cytoplasmic domain.

In another particular embodiment, an anti-KIT antibody or an antigen binding fragment thereof for use in methods provided herein inhibits cell proliferation, for example, mast cell proliferation or eosinophil proliferation. In yet another particular embodiment, an anti-KIT antibody or an antigen binding fragment thereof for use in methods provided herein inhibits cell survival, for example mast cell survival or eosinophil cell survival. In certain aspects, inhibition of cell proliferation, for example, mast cell proliferation or eosinophil proliferation, is at least 10%, 20%, 30%, 40%, 50%, 60%, 70%, 80%, or 90%.

In another particular embodiment, an anti-KIT antibody or an antigen binding fragment thereof for use in methods provided herein inhibits mast cell activation or eosinophil activation. In certain aspects, inhibition of mast cell activation or activity or eosinophil activation or activity, is at least 10%, 20%, 30%, 40%, 50%, 60%, 70%, 80%, or 90%.

In a specific embodiment, an anti-KIT antibody or an antigen binding fragment thereof for use in methods provided herein inhibits eosinophil or mast cell degranulation (see, e.g., Staats et al., 2012, Med. Chem. Commun., 2013, 4:88-94; and Ochkur et al., 2012, J. Immunol. Methods, 384: 10-20). In certain aspects, inhibition of eosinophil or mast cell degranulation is at least 10%, 20%, 30%, 40%, 50%, 60%, 70%, 80%, or 90%.

In another particular embodiment, an anti-KIT antibody or an antigen binding fragment thereof for use in methods provided herein inhibits mast cell mediator release. In certain aspects, mast cell mediator release is at least 10%, 20%, 30%, 40%, 50%, 60%, 70%, 80%, or 90%. Assays to measure mast cell activity, such as release of mediators, from mast cell cultures, such as rodent and human mast cell cultures, have been described (see, e.g., Kuehn et al., “Measuring Mast Cell Mediator Release,” in Current Protocols in Immunology, Unite 7.38.1-7.38.9, November 2010 (John Wiley & Sons, Inc.). In certain aspects, CD34 peripheral blood progenitor cells or a mast cell line, such as HMC-1 or human LAD2 mast cell line can be used in these assays to ascertain the effects of an anti-KIT antibody on mast cells.

In a specific embodiment, an anti-KIT antibody or an antigen binding fragment thereof for use in methods provided herein induces apoptosis, for example mast cell apoptosis or eosinophil apoptosis. In another specific embodiment, an anti-KIT antibody or an antigen binding fragment thereof for use in methods provided herein induces cell differentiation, e.g., mast cell differentiation.

In a particular embodiment, an anti-KIT antibody or an antigen binding fragment thereof for use in methods provided herein can achieve any one of the following: reduction in the number and/or activity of eosinophils, reduction in mast cell proliferation, reduction in mast cell number or amount, inhibition or reduction in mast cell activity, reduction in mast cell induced production or release of inflammatory factors, reduction in release of inflammatory factors, restoration of mast cell homeostasis, reduced mast cell migration, reduced mast cell adhesion, inhibition or reduction in mast cell recruitment of eosinophils, and inhibition or reduction in antigen-mediated degranulation of mast cells.

In a particular embodiment, an anti-KIT antibody or an antigen binding fragment thereof for use in methods provided herein inhibits KIT activity but does not inhibit KIT dimerization. In another particular embodiment, an anti-KIT antibody or an antigen binding fragment thereof for use in methods provided herein inhibits KIT activity and does not inhibit ligand binding to KIT, e.g., does not inhibit KIT ligand (e.g., SCF) binding to KIT, but does inhibit KIT dimerization.

In a particular embodiment, an anti-KIT antibody or an antigen binding fragment thereof for use in methods provided herein inhibits a KIT activity, such as ligand-induced tyrosine phosphorylation of a KIT cytoplasmic domain, by about 25% to about 65% or 75%, as determined by a cell-based phosphorylation assay well known in the art, for example, the cell-based phosphorylation assay described herein. In a certain embodiment, an anti-KIT antibody or an antigen binding fragment thereof for use in methods provided herein inhibits a KIT activity, such as ligand-induced tyrosine phosphorylation of a KIT cytoplasmic domain, by about 35% to about 85% or 95%, as determined by a cell-based phosphorylation assay well known in the art, for example, the cell-based phosphorylation assay described herein.

In a particular embodiment, an anti-KIT antibody or an antigen binding fragment thereof for use in methods provided herein inhibits a KIT activity, such as ligand-induced tyrosine phosphorylation of a KIT cytoplasmic domain, with a 50% inhibition concentration (IC₅₀) of less than about 600 pM, or less than about 500 pM, or less than about 250 pM, as determined by a cell-based phosphorylation assay well known in the art, for example, the cell-based phosphorylation assay described herein. In a specific embodiment, the IC₅₀ is less than about 550 pM or 200 pM. In a specific embodiment, the IC₅₀ is in the range of about 50 pM to about 225 pM, or in the range of 100 pM to about 600 pM. In a specific embodiment, the IC₅₀ is in the range of about 50 pM to about 550 pM, or about 50 pM to about 600 pM, or about 150 pM to about 550 pM.

In a specific embodiment, an anti-KIT antibody or an antigen binding fragment thereof for use in methods provided herein, (i) immunospecifically binds to a KIT polypeptide comprising the D4 and/or D5 region of human KIT, (ii) inhibits KIT phosphorylation (e.g., tyrosine phosphorylation), and (iii) does not fully inhibit, or partially inhibits, KIT ligand (e.g., SCF) binding to KIT. In yet another specific embodiment, such an antibody does not inhibit KIT dimerization. In yet another specific embodiment, such an antibody can be recombinantly expressed by CHO cells at an average titer of at least 0.5 μg/mL, for example at least 1.0 μg/mL. In a further specific embodiment, such an antibody comprises a VH domain and a VL domain that are non-immunogenic, for example, the VH domain and VL domain do not contain T cell epitopes.

In other specific embodiments, an anti-KIT antibody or an antigen binding fragment thereof for use in methods provided herein immunospecifically binds to a monomeric form of KIT (e.g., human KIT). In particular embodiments, an anti-KIT antibody or an antigen binding fragment thereof for use in methods provided herein specifically bind to a monomeric form of KIT (e.g., human KIT). In specific embodiments, an anti-KIT antibody or an antigen binding fragment thereof for use in methods provided herein specifically binds to a dimeric form of KIT (e.g., human KIT).

In specific embodiments, an anti-KIT antibody or an antigen binding fragment thereof for use in methods provided herein does not bind to a monomeric form of KIT and specifically binds to a dimeric form of KIT or multimeric form of KIT. In certain embodiments, an antibody has higher affinity for a KIT monomer than a KIT dimer. In certain embodiments, an antibody has higher affinity for a KIT monomer than a KIT multimer.

In specific embodiments, an anti-KIT antibody or an antigen binding fragment thereof for use in methods provided herein specifically binds to a native isoform or native variant of KIT (that is a naturally occurring isoform or variant of KIT in an animal (e.g., monkey, mouse, goat, donkey, dog, cat, rabbit, pig, rat, human, frog, or bird) that can be isolated from an animal, preferably a human). In particular embodiments, an anti-KIT antibody or an antigen binding fragment thereof for use in methods provided herein specifically binds to human KIT or a fragment thereof. In specific embodiments, an anti-KIT antibody or an antigen binding fragment thereof for use in methods provided herein specifically binds to human KIT or a fragment thereof and does not specifically bind to a non-human KIT (e.g. monkey, mouse, goat, donkey, dog, cat, rabbit, pig, rat, or bird) or a fragment thereof. In specific embodiments, an anti-KIT antibody or an antigen binding fragment thereof for use in methods provided herein specifically binds to human KIT or a fragment thereof and does not specifically bind to murine KIT. In certain embodiments, an anti-KIT antibody or an antigen binding fragment thereof for use in methods provided herein specifically binds to human KIT or a fragment thereof (e.g., a D4 region of human KIT) and to canine (dog) and non-human primate (e.g., monkey) KIT. In certain embodiments, an anti-KIT antibody or an antigen binding fragment thereof for use in methods provided herein specifically binds to human KIT or a fragment thereof (e.g., a D4 region of human KIT) and to canine (dog) and non-human primate (e.g., monkey) KIT, but does not specifically bind to murine or rat KIT or a fragment thereof (e.g., a D4 region of murine KIT).

In certain embodiments, an anti-KIT antibody or an antigen binding fragment thereof for use in methods provided herein specifically binds to human KIT or a fragment thereof (e.g. a D4 region of human KIT) and to canine (dog), feline (cat) and cynomologous KIT, but does not specifically bind to murine or rat KIT or a fragment thereof (e.g., a D4 region of murine KIT).

In specific embodiments, an anti-KIT antibody or an antigen binding fragment thereof for use in methods provided herein specifically binds to human KIT or a fragment thereof (e.g. a D4 region of human KIT), and to canine (dog), feline (cat) and cynomologous KIT, with high affinity (e.g., at least 0.5 fold, 1 fold, 2 fold, 3 fold, 4 fold, 5 fold, or 10 fold) than to murine or rat KIT or a fragment thereof (e.g., a D4 region of murine KIT).

In certain embodiments, an anti-KIT antibody or an antigen binding fragment thereof for use in methods provided herein specifically binds to an extracellular domain of human KIT comprising a mutation, for example a somatic mutation, such as a mutation in exon 9 of human KIT wherein the Ala and Tyr residues at positions 502 and 503 are duplicated (see, e.g., Marcia et al., (2000) Am. J. Pathol. 156(3):791-795; and Debiec-Rychter et al., (2004) European Journal of Cancer. 40:689-695, which are both incorporated herein by reference in their entireties, describing KIT mutations).

In certain embodiments, an anti-KIT antibody or an antigen binding fragment thereof for use in methods provided herein specifically binds to an extracellular domain of human KIT which is glycosylated. In certain embodiments, an antibody described herein or antigen-binding fragment thereof binds to two different glycosylated forms of an extracellular domain of human KIT. For example, two forms of human KIT with different molecular weights, indicating different glycosylation patterns, have been observed by immunoblotting.

In certain embodiments, an antibody described herein may specifically bind to both of these forms of human KIT which have different glycosylation patterns, e.g., one form is more glycosylated than the other. In certain embodiments, an antibody described herein or antigen-binding fragment thereof binds to an extracellular domain of human KIT which is not glycosylated.

In a specific embodiment, an anti-KIT antibody or antigen binding fragment thereof for use in the methods provided herein is a bivalent monospecific antibody, in that it has two antigen binding regions (e.g., two identical antigen binding regions) and both antigen binding regions specifically bind the same antigen, KIT (e.g., human KIT). In certain embodiments, the antigen binding region comprises the VH and VL CDRs as set forth in Table 1. In particular embodiments, the antigen binding region comprises a VH comprising the amino acid sequence of any one of SEQ ID NOs: 8-12, and/or a VL comprising the amino acid sequence of any one of SEQ ID NOs: 13-16. In certain aspects, an anti-KIT antibody or antigen binding fragment thereof for use in the methods provided herein is not a bispecific antibody.

In a particular embodiment, an anti-KIT antibody for use in the methods provided herein is a Fab fragment that immunospecifically binds to a KIT polypeptide, such as the D4 region of KIT. In a specific embodiment, antibodies for use in the methods described herein are monoclonal antibodies or isolated monoclonal antibodies. In another specific embodiment, an antibody for use in the methods described herein is a humanized monoclonal antibody. In a particular embodiment, an antibody for use in the methods described herein is a recombinant antibody, for example, a recombinant human antibody, recombinant humanized antibody or a recombinant monoclonal antibody. In certain embodiments, an antibody for use in the methods described herein contains non-human amino acid sequences, e.g., non-human CDRs or non human (e.g., non-human primate) framework residues.

In particular embodiments provided herein, recombinant antibodies can be isolated, prepared, expressed, or created by recombinant means, such as antibodies expressed using a recombinant expression vector transfected into a host cell, antibodies isolated from a recombinant, combinatorial antibody library, or antibodies prepared, expressed, created or isolated by any other means that involves creation, e.g., via synthesis, genetic engineering of DNA sequences that encode human immunoglobulin sequences, or splicing of sequences that encode human immunoglobulins, e.g., human immunoglobulin gene sequences, to other such sequences. In certain embodiments, the amino acid sequences of such recombinant antibodies have been modified such thus the amino acid sequences of such antibodies, e.g., VH and/or VL regions, are sequences that do not naturally exist within an organism's antibody germline repertoire in vivo, for example a murine or human germline repertoire. In a particular embodiment, a recombinant antibody can be obtained by assembling several sequence fragments that naturally exist in an organism (e.g., primate, such as human) into a composite sequence of a recombinant antibody, wherein the composite sequence does not naturally exist within an organism (e.g., primate such as human).

Antibodies for use in the methods provided herein include immunoglobulin molecules of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody for use in the methods provided herein is an IgG antibody (e.g., human IgG antibody), or a class (e.g., human IgG1 or IgG4) or subclass thereof. In another specific embodiment, an antibody for use in the methods described herein is an IgG1 (e.g., human IgG1 (isotype a, z, or f)) or IgG4 antibody. In certain embodiments, an antibody for use in the methods described herein is a whole or entire antibody, e.g., a whole or entire humanized, human, or composite human antibody.

In specific aspects, the antibody provided herein comprises an antibody light chain and heavy chain, e.g., a separate light chain and heavy chain. With respect to the light chain, in a specific embodiment, the light chain of an antibody described herein is a kappa light chain. In another specific embodiment, the light chain of an antibody described herein is a lambda light chain. In yet another specific embodiment, the light chain of an antibody described herein is a human kappa light chain or a human lambda light chain. In a particular embodiment, an antibody described herein comprises a human light chain constant region. Non-limiting examples of human light chain constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

With respect to the heavy chain, in a specific embodiment, the heavy chain of an antibody described herein can be an alpha (α), delta (δ), epsilon (ε), gamma (γ) or mu (μ) heavy chain. In another specific embodiment, the heavy chain of an antibody described can comprise a human alpha (α), delta (δ), epsilon (ε), gamma (γ) or mu (μ) heavy chain. In a particular embodiment, an antibody described herein comprises a human heavy chain constant region (e.g., a human IgG constant region, for example, a human IgG1, IgG2, IgG3, or IgG4 constant region). Non-limiting examples of human heavy chain constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242. In a specific embodiment, the antibody described herein comprises a modified (e.g., mutated) human Fc region or domain (e.g., a modified (e.g., mutated) human IgG1 Fc region or domain, a modified (e.g., mutated) human IgG2 Fc region or domain, a modified (e.g., mutated) human IgG3 Fc region or domain, or a modified (e.g., mutated) human IgG4 Fc region or domain).

Antibodies provided herein can include antibody fragments that retain the ability to specifically bind to an antigen, e.g., KIT epitope (e.g., a KIT epitope within a KIT polypeptide containing a D4 region of human KIT). In a specific embodiment, fragments include Fab fragments (an antibody fragment that contains the antigen-binding domain and comprises a light chain and part of a heavy chain (i.e., the VH and CHI domains of a heavy chain) bridged by a disulfide bond); Fab′ (an antibody fragment containing a single antigen-binding domain comprising an Fab and an additional portion of the heavy chain through the hinge region); F(ab′)₂ (two Fab′ molecules joined by interchain disulfide bonds in the hinge regions of the heavy chains; the Fab′ molecules can be directed toward the same or different epitopes); a bispecific Fab (a Fab molecule having two antigen binding domains, each of which can be directed to a different epitope); a single chain Fab chain comprising a variable region, also known as a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which can be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody can be directed towards the same or different epitopes); and a triabody (a trimerized sFv, formed in a manner similar to a diabody, but in which three antigen-binding domains are created in a single complex; the three antigen binding domains can be directed towards the same or different epitopes). Antibodies provided herein can also include one or more CDR sequences of an antibody. The CDR sequences can be linked together on a scaffold when two or more CDR sequences are present. In certain embodiments, an antibody comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs, see Pluckthun in The Pharmacology of Monoclonal Antibodies, vol. 113, Rosenburg and Moore eds. Springer-Verlag, New York, pp. 269-315 (1994). Without being bound by any particular theories, Fv molecules can be able to penetrate tissues because of their small size. A whole antibody can be enzymatically cleaved by pepsin to produce a F(ab′)₂ fragment, or can be enzymatically cleaved by papain to produce two Fab fragments. The antibody or antigen-binding fragment thereof may also be linked to a further binding entity, such as an SCF binding sequence or “trap”.

In certain embodiments, anti-KIT antibodies for use in the methods described herein are human, composite human, or humanized monoclonal antibodies. In a particular embodiment, an antibody for use in the methods described herein is an engineered antibody, for example, antibody produced by recombinant methods. In a specific embodiment, an antibody described herein is a humanized antibody comprising one or more non-human (e.g. rodent or murine) CDRs and one or more human framework regions (FR), and optionally human heavy chain constant region and/or light chain constant region. In a specific embodiment, an antibody described herein comprises one or more primate (or non-human primate) framework regions. In a specific embodiment, an antibody described herein does not comprise non-human primate framework regions.

Antibodies for use in the methods provided herein can include antibodies comprising chemical modifications, for example, antibodies which have been chemically modified, e.g., by covalent attachment of any type of molecule to the antibody. For example, but not by way of limitation, an anti-KIT antibody can be glycosylated, acetylated, pegylated, phosphorylated, or amidated, can be derivitized via protective/blocking groups, or can further comprise a cellular ligand and or other protein or peptide (e.g., a heterologous protein or peptide), etc. For example, an antibody provided herein can be chemically modified, e.g., by glycosylation, acetylation, pegylation, phosphorylation, amidation, derivatization by known protecting/blocking groups, proteolytic cleavage, linkage to a cellular ligand or other protein, etc. Further, an anti-KIT antibody described herein can contain one or more non-classical amino acids. In some embodiments, provided herein are conjugates comprising an anti-KIT antibody or antigen binding fragment thereof linked to another agent (e.g., a therapeutic agent). Such antibody conjugates may also be used in a method or use described in this disclosure.

In one embodiment, an anti-KIT antibody for use in the methods provided herein is a naked antibody which is not linked, fused or conjugated (e.g. artificially linked, fused or conjugated) to another molecule, peptide or polypeptide (for example, a heterologous polypeptide). In a particular embodiment, an anti-KIT antibody for use in the methods provided herein is not an antibody-drug conjugate. In a particular embodiment, an anti-KIT antibody for use in the methods provided herein is not a fusion protein. In particular embodiments, an anti-KIT antibody described herein does not comprise any non-classical amino acids.

### 5.2 Antibody Production

Antibodies (e.g., human or humanized antibodies) described herein (or an antigen-binding fragment thereof) that immunospecifically bind to a KIT antigen can be produced by any method known in the art for the synthesis of antibodies, for example, by chemical synthesis or by recombinant expression techniques. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et at, Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

For example, humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169: 1119 25 (2002), Caldas et al., Protein Eng. 13(5):353-60 (2000), Morea et al., Methods 20(3):267 79 (2000), Baca et al., J. Biol. Chem.272(16): 10678-84 (1997), Roguska et al., Protein Eng. 9(10):895 904 (1996), Couto et al., Cancer Res. 55 (23 Supp):5973s-5977s (1995), Couto et al., Cancer Res. 55(8): 1717-22 (1995), Sandhu JS, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated herein by reference.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced by recombinant technology, e.g. recombinant monoclonal antibodies expressed by a host cell, such as a mammalian host cell.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., extracellular domain of human KIT) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). Additionally, a RIMMS (repetitive immunization multiple sites) technique can be used to immunize an animal (Kilptrack et al., 1997 Hybridoma 16:381-9, which is incorporated herein by reference).

Non-limiting examples of myeloma cell lines include murine myeloma lines, such as those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Antibodies described herein include antibody fragments which recognize specific KIT antigens and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CHI domains of the heavy chain. A F(ab′)₂ fragment contains the two antigen-binding arms of an antibody molecule linked by disulfide bonds in the hinge region.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1 (3):253-263; Muylderman, 2001, J. iotechnol. 74(4):277302; U.S. Pat. No. 6,005,079; and International Publication Nos. WO 94/04678, WO 94/25591, and WO 01/44301.

### 5.3 Compositions

Provided herein are compositions, such as pharmaceutical compositions, comprising one or more anti-KIT antibodies (e.g., humanized antibodies) or antigen-binding fragments thereof for use in the methods described herein. In particular aspects, compositions described herein can be for in vitro, in vivo, or ex vivo uses. In specific embodiments, provided herein is a pharmaceutical composition comprising an anti-KIT antibody (e.g., a humanized antibody) or an antigen-binding fragment thereof for use in the methods described herein and a pharmaceutically acceptable carrier or excipient.

As used herein, the term “pharmaceutically acceptable” means being approved by a regulatory agency of the Federal or a state government, or listed in the U.S. Pharmacopeia, European Pharmacopeia or other generally recognized Pharmacopeia for use in animals, and more particularly in humans.

Therapeutic formulations containing one or more antibodies (e.g., humanized antibodies) provided herein can be prepared for storage by mixing the antibody having the desired degree of purity with optional physiologically acceptable carriers, excipients or stabilizers (Remington's Pharmaceutical Sciences (1990) Mack Publishing Co., Easton, PA; Remington: The Science and Practice of Pharmacy, 21st ed. (2006) Lippincott Williams & Wilkins, Baltimore, MD), in the form of lyophilized formulations or aqueous solutions. Acceptable carriers, excipients, or stabilizers are nontoxic to recipients at the dosages and concentrations employed, and include buffers such as phosphate, citrate, and other organic acids; and/or non-ionic surfactants such as TWEEN™, PLURONICS™ or polyethylene glycol (PEG).

Formulations, such as those described herein, can also contain more than one active compounds (for example, molecules, e.g., antibody or antibodies described herein) as necessary for the particular indication being treated. In certain embodiments, formulations comprise an antibody provided herein and one or more active compounds with complementary activities that do not adversely affect each other. Such molecules are suitably present in combination in amounts that are effective for the purpose intended.

The formulations to be used for in vivo administration can be sterile. This is readily accomplished by filtration through, e.g., sterile filtration membranes.

In specific aspects, the pharmaceutical compositions provided herein contain therapeutically effective amounts of one or more of the anti-KIT antibodies (e.g., humanized antibodies) provided herein, and optionally one or more additional prophylactic of therapeutic agents, in a pharmaceutically acceptable carrier. Such pharmaceutical compositions are useful in the prevention, treatment, management or amelioration of chronic prurigo, or one or more of the symptoms thereof.

Pharmaceutical carriers suitable for administration of the antibodies provided herein include any such carriers known to those skilled in the art to be suitable for the particular mode of administration.

In addition, the antibodies described herein can be formulated as the sole pharmaceutically active ingredient in the composition or can be combined with other active ingredients (such as one or more other prophylactic or therapeutic agents).

Compositions can contain one or more anti-KIT antibodies provided herein. In one embodiment, the antibodies are formulated into suitable pharmaceutical preparations, such as solutions, suspensions, powders, or elixirs, in sterile solutions or suspensions for parenteral administration. In one embodiment, the antibodies are formulated into suitable pharmaceutical preparations, such as solutions, suspensions, tablets, dispersible tablets, pills, capsules, powders, sustained release formulations or elixirs, for oral administration, as well as transdermal patch preparation and dry powder inhalers.

In such compositions, one or more antibodies provided herein (or conjugates thereof) is (are) mixed with a suitable pharmaceutical carrier. Concentrations of an antibody or antibodies in the compositions can, for example, be effective for delivery of an amount, upon administration, that treats, prevents, protects against or manages chronic prurigo, or one or more symptoms thereof.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

In certain aspects, an antibody (e.g., a humanized antibody) provided herein (or an antibody-drug conjugate thereof) is included in the pharmaceutically acceptable carrier in an effective amount sufficient to exert a therapeutically useful effect in the absence of, or with minimal or negligible, undesirable side effects on the patient treated. A therapeutically effective concentration can be determined empirically by testing the compounds in in vitro and in vivo systems using routine methods and then extrapolated therefrom for dosages for humans.

The concentration of antibody in the pharmaceutical composition will depend on, e.g., the physicochemical characteristics of the antibody, the dosage schedule, and amount administered as well as other factors known to those of skill in the art. In certain aspects, the concentration of antibody-drug conjugate in the pharmaceutical composition will depend on, e.g., the physicochemical characteristics of the antibody and/or the drug, the dosage schedule, and amount administered as well as other factors known to those of skill in the art.

In one embodiment, a therapeutically effective dosage produces a serum concentration of antibody of from about 0.1 ng/ml to about 50-100 pg/ml. The pharmaceutical compositions, in another embodiment, provide a dosage of from about 0.001 mg to about 2000 mg of antibody per kilogram of body weight for administration over a period of time, e.g., every day, every week, every 2 weeks, or every 3, 4 or 8 weeks. Pharmaceutical dosage unit forms can be prepared to provide from about 0.01 mg to about 2000 mg, and in one embodiment from about 10 mg to about 500 mg of the antibody and/or a combination of other optional essential ingredients per dosage unit form.

In a particular embodiment, an antibody-drug conjugate described herein is administered at an effective dosage of about 1 to 100 mg of antibody-drug conjugate per kilogram of body weight for administration over a period of time, e.g., every day, every week, every 2 weeks, or every 3 weeks.

An anti-KIT antibody described herein can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

Upon mixing or addition of an antibody, the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and can be empirically determined.

Pharmaceutical compositions described herein are provided for administration to humans and animals, such as mammals (e.g., cat or dog), in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals, such as mammals (e.g., cat or dog), in unit dosage form, such as tablets, capsules, pills, powders, granules, and oral solutions or suspensions, and oil-water emulsions containing suitable quantities of the compounds or pharmaceutically acceptable derivatives thereof. The antibody is, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the antibody sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

In certain embodiments, one or more anti-KIT antibodies described herein are in a liquid pharmaceutical formulation. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington's Pharmaceutical Sciences (1990) Mack Publishing Co., Easton, PA; Remington: The Science and Practice of Pharmacy, 21st ed. (2006) Lippincott Williams & Wilkins, Baltimore, MD.

Dosage forms or compositions containing antibody in the range of 0.005% to 100% with the balance made up from non-toxic carrier can be prepared. Methods for preparation of these compositions are known to those skilled in the art.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents. Other routes of administration may include, epidural administration, enteric administration, intracerebral administration, nasal administration, intraarterial administration, intracardiac administration, intraosseous infusion, intrathecal administration, and intraperitoneal administration. In a specific embodiment, an anti-KIT antibody, antigen binding fragment, or pharmaceutical composition described herein is administered intravenously. In a specific embodiment, an anti-KIT antibody, antigen binding fragment, or pharmaceutical composition described herein is administered subcutaneously.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

An anti-KIT antibody described herein can be suspended in micronized or other suitable form. The form of the resulting mixture depends upon a number of factors, including the intended mode of administration and the solubility of the compound in the selected carrier or vehicle. The effective concentration is sufficient for ameliorating the symptoms of the condition and can be empirically determined.

In other embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving an antibody provided herein, in a suitable solvent. In some embodiments, the lyophilized powder is sterile. The solvent can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Antibodies described herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

The antibodies and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, the anti-KIT antibodies described herein are targeted (or otherwise administered) to the bone marrow. In some embodiments, anti-KIT antibodies described herein are targeted (or otherwise administered) to the gastrointestinal tract. In some embodiments, anti-KIT antibodies described herein are targeted (or otherwise administered) to the brain. In specific embodiments, an anti-KIT antibody described herein is capable of crossing the blood-brain barrier.

In specific embodiments, anti-KIT antibodies described herein are targeted (or otherwise administered) to an ocular tissue or organ. In particular aspects, a composition comprising anti-KIT antibodies described herein can be targeted to an ocular tissue or organ as eye drops or gels. In particular aspects, a composition comprising anti-KIT antibodies described herein can be targeted to the ear.

Provided herein is a pharmaceutical pack or kit comprising one or more containers filled with one or more of the ingredients of the pharmaceutical compositions described herein, such as one or more antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

For KIT inhibitors other than antibodies, it is contemplated that they also can be formulated in pharmaceutical formulations with pharmaceutically acceptable carriers suitable for storage, handling, and/or administration.

### 5.4 Dosages and Administration

The dosage and frequency of administration of an anti-KIT antibody described herein or a pharmaceutical composition thereof is administered to a subject in need thereof (e.g., mammal, such as human, dog or cat) in accordance with the methods for treating chronic prurigo provided herein will be efficacious while minimizing side effects. The exact dosage of an anti-KIT antibody described herein to be administered to a particular subject or a pharmaceutical composition thereof can be determined in light of factors related to the subject that requires treatment. Factors which can be taken into account include the severity of the disease state, general health of the subject, age, and weight of the subject, diet, time and frequency of administration, combination(s) with other therapeutic agents or drugs, reaction sensitivities, and tolerance/response to therapy. The dosage and frequency of administration of an anti-KIT antibody described herein or a pharmaceutical composition thereof can be adjusted over time to provide sufficient levels of the anti-KIT antibody or to maintain the desired effect.

The precise dose to be employed in the formulation will also depend on the route of administration, and the seriousness of the disorder or disease and should be decided according to the judgment of the practitioner and each patient's circumstances.

In certain aspects, for the anti-KIT antibodies described herein, the dosage administered to a patient, to prevent, protect against, manage, or treat chronic prurigo is typically 0.1 mg/kg to 100 mg/kg of the patient's body weight. In a specific embodiment, the dosage administered to a patient, to prevent, protect against, manage, or treat chronic prurigo is about 3 mg/kg of the patient's body weight.

Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies described herein can be reduced by enhancing uptake and tissue penetration of the antibodies by modifications such as, for example, lipidation.

In one embodiment, approximately 0.001 mg/kg (mg of antibody per kg weight of a subject) to approximately 500 mg/kg of an anti-KIT antibody described herein is administered to prevent, protect against, manage, or treat chronic prurigo. In another embodiment, approximately 0.3 mg/kg (mg of antibody per kg weight of a subject) of an anti-KIT antibody described herein is administered to prevent, protect against, manage, or treat chronic prurigo. In another embodiment, approximately 1 mg/kg (mg of antibody per kg weight of a subject) of an anti-KIT antibody described herein is administered to prevent, protect against, manage, or treat chronic prurigo. In another embodiment, approximately 3 mg/kg (mg of antibody per kg weight of a subject) of an anti-KIT antibody described herein is administered to prevent, protect against, manage, or treat chronic prurigo. In another embodiment, approximately 9 mg/kg (mg of antibody per kg weight of a subject) of an anti-KIT antibody described herein is administered to prevent, protect against, manage, or treat chronic prurigo.

In some embodiments, an effective amount of an antibody provided herein is from about 0.01 mg to about 1,000 mg. In specific embodiments, an “effective amount” or “therapeutically effective amount” of an anti-KIT antibody described herein refers to an amount of an anti-KIT antibody described herein which is sufficient to achieve at least one, two, three, four or more of the following effects: the reduction or amelioration of the severity of chronic prurigo, and/or one or more symptoms associated therewith; the reduction in the duration of one or more symptoms associated with chronic prurigo; the prevention in the recurrence of one or more symptoms of chronic prurigo, and/or one or more symptoms associated therewith; the reduction in hospitalization of a subject; the reduction in hospitalization length; the inhibition (e.g., partial inhibition) of the progression of chronic prurigo, and/or one or more symptoms associated therewith; the prevention of the development or onset of one or more symptoms associated with chronic prurigo; a decrease in the concentration of one or more inflammatory mediators (e.g., cytokines or interleukins) in biological specimens (e.g., plasma, serum, cerebral spinal fluid, urine, or any other bio fluids) of a subject with chronic prurigo; and improvement in the quality of life as assessed by methods well known in the art, e.g., questionnaires. In some embodiments, “effective amount” as used herein also refers to the amount of an antibody described herein to achieve a specified result (e.g., inhibition of one or more KIT biological activities of a cell, such as inhibition of cell proliferation).

In some embodiments, an anti-KIT antibody described herein is administered as necessary, e.g., weekly, biweekly (i.e., once every two weeks), monthly, bimonthly, trimonthly, etc.

In some embodiments, a single dose of an anti-KIT antibody described herein is administered one or more times to a patient to impede, prevent, manage, treat and/or ameliorate chronic prurigo.

In particular embodiments, an anti-KIT antibody or pharmaceutical composition thereof is administered to a subject in accordance with the methods for treating chronic prurigo, provided herein in cycles, wherein the anti-KIT antibody or pharmaceutical composition is administered for a period of time, followed by a period of rest (i.e., the anti-KIT antibody) or pharmaceutical composition is not administered for a period of time).

The methods provided herein involve administering an anti-KIT antibody by any suitable route. Non-limiting examples of routes of administration include, parenteral administration for example subcutaneous, intramuscular or intravenous administration, epidural administration, enteric administration, intracerebral administration, nasal administration, intraarterial administration, intracardiac administration, intraosseous infusion, intrathecal administration, and intraperitoneal administration. Methods provided herein include routes of administration targeting the brain, an ocular tissue or organ, spinal cord, or ear or auricular tissue. In a particular aspect, methods provided herein include routes of administration targeting the nervous system, e.g., central nervous system.

In specific embodiments, methods provided herein involve administering an anti-KIT antibody via a route suitable for crossing the blood-brain barrier.

For KIT inhibitors other than antibodies, it is contemplated that the above description about dosage and administration also applies to the extent deemed appropriate by the treating physician.

### 6. EXAMPLES

The examples herein are offered by way of illustration, and not by way of limitation.

### 6.1 Example 1

Nucleic acid molecules encoding the Fc mutated H (heavy chain) and L (light chain) amino acid sequences of an anti-KIT antibody (SEQ ID NO: 23 and SEQ ID NO: 24 respectively, see Table 6 below) were cloned directly into an expression vector. Constructs were confirmed by sequencing and the vector was transfected into CHO cells. Stable transfections to establish cell lines expressing the antibody were carried out and were subsequently drug-selected. The best expressing lines were selected based on an above-background IgG titre in the supernatant, and expanded in the presence of drug selection with screening for IgG expression using an Octet® QKe system at every stage.

TABLE 6
DNA Sequences encoding heavy and light chain sequences of an anti-KIT antibody.

| | |
|---|---|
| Heavy | atggagtggtcctgggtgttcctgttctttctgtccgtgaccacaggcgtgcacagccaggtgcagctggtgcagtc |
| Chain | tggagctgaggtgaagaagccaggagcttctgtgaagctgtcctgcaaggccagcggctacaccttcacagacta |
| Sequence | ctatatcaactgggtgagacaggctcctggcaagggcctggagtggatcgctcgcatctatccaggctctggcaa |
| (DNA): | cacctactataatgagaagtttaagggccgggccaccctgacagctgataagagcacctctacagcttacatgcag |
| | ctgtccagcctgagatccgaggacaccgccgtgtacttctgcgctcgcggcgtgtactattttgattattggggcca |
| | gggcaccacagtgaccgtgtcttccgctagcacaaagggcccttccgtgtttccactggctcccagctctaagtcc |
| | accagcggaggaacagccgctctgggctgtctggtgaaggactatttcccagagcccgtgaccgtgagctggaa |
| | ctctggcgccctgaccagcggagtgcatacatttcctgctgtgctgcagtccagcggcctgtactctctgtcttccgt |
| | ggtgaccgtgccaagctcttccctgggcacccagacatatatctgcaacgtgaatcacaagccatccaatacaaag |
| | gtggacaagaaggtggagcccaagagctgtgataagacccatacatgccccccttgtcctgctccagaggctca |
| | gggaggaccatccgtgttcctgtttccacccaagcctaaggacaccctgtacatcacaagggagccagaggtgac |
| | ctgcgtggtggtggacgtgagccacgaggatcccgaggtgaagttcaactggtacgtggatggcgtggaggtgc |
| | ataatgccaagacaaagccaagggaggagcagtacaatagcacctatcgggtggtgtctgtgctgacagtgctgc |
| | accaggactggctgaacggcaaggagtacaagtgccaggtgtctaataaggccctgcccgctcctatcgagaag |
| | accatctccaaggccaagggccagcctagggagccacaggtgtacacactgcctccaagccgggacgagctga |
| | ccaagaaccaggtgtctctgacatgtctggtgaagggcttctatccctctgatatcgctgtggagtgggagtccaat |
| | ggccagcctgagaacaattacaagaccacaccccctgtgctggactccgatggcagcttctttctgtattccaagct |
| | gaccgtggataagagcaggtggcagcagggcaacgtgttttcttgttccgtgatgcatgaggctctgcacaatcatt |
| | acacacagaagagcctgtctctgtcccctggc (SEQ ID NO: 23). |
| Light | atgtccgtgccaacccaggtgctgggactgctgctgctgtggctgaccgacgccaggtgcgatatcgtgatgaca |
| Chain | cagtccccttccagcctgtctgcttccgtgggcgacagagtgaccatcacctgtaaggccagccagaacgtgcgc |
| Sequence | accaatgtggcttggtaccagcagaagccaggcaaggcccccaaggctctgatctatagcgcctcttacaggtat |
| (DNA) | agcggagtgcctgaccggttcaccggatccggaagcggaacagacttcaccctgacaatctcttccctgcagcct |
| | gaggacttcgctgattacttttgccagcagtacaactcttatccaaggaccttcggcggcggcacaaaggtggaga |
| | tcaagcggaccgtggccgctccaagcgtgttcatctttcccccttctgacgagcagctgaagtctggcacagcctc |
| | cgtggtgtgcctgctgaacaacttctaccccagagaggccaaggtgcagtggaaggtggataacgctctgcagtc |
| | tggcaattcccaggagagcgtgaccgagcaggactctaaggattccacatatagcctgagctctaccctgacact |
| | gtctaaggccgattacgagaagcacaaggtgtatgcttgcgaggtgacccatcagggcctgtccagcccagtgac |
| | aaagtccttcaatcgcggcgagtgt (SEQ ID NO: 24). |

The resulting antibody after removal of the leader sequences was designated antibody mAb1. The heavy and light chain amino acid sequences of the antibody (after removal of the leader sequences) are set forth in Table 7 below.

TABLE 7
Full-length heavy and light chain amino acid sequences of mAb1.

| | |
|---|---|
| mAb1 | QVQLVQSGAEVKKPGASVKLSCKASGYTFTDYYINWVRQAPGKGLEWI |
| full-length | ARIYPGSGNTYYNEKFKGRATLTADKSTSTAYMQLSSLRSEDTAVYFCA |
| heavy | RGVYYFDYWGQGTTVTVSSASTKGPSVFPLAPSSKSTSGGTAALGCLV |
| chain | KDYFPEPVTVSWNSGALTSGVHTFPAVLQSSGLYSLSSVVTVPSSSLGTQ |
| amino | TYICNVNHKPSNTKVDKKVEPKSCDKTHTCPPCPAPEAQGGPSVFLFPP |
| acid | KPKDTLYITREPEVTCVVVDVSHEDPEVKFNWYVDGVEVHNAKTKPRE |
| sequence | EQYNSTYRVVSVLTVLHQDWLNGKEYKCQVSNKALPAPIEKTISKAKG |
| (including | QPREPQVYTLPPSRDELTKNQVSLTCLVKGFYPSDIAVEWESNGQPENN |
| Fc): | YKTTPPVLDSDGSFFLYSKLTVDKSRWQQGNVFSCSVMHEALHNHYTQ |
| | KSLSLSPG (SEQ ID NO: 21) |
| mAb1 | DIVMTQSPSSLSASVGDRVTITCKASQNVRTNVAWYQQKPGKAPKALIY |
| full-length | SASYRYSGVPDRFTGSGSGTDFTLTISSLQPEDFADYFCQQYNSYPRTFG |
| light chain | GGTKVEIKRTVAAPSVFIFPPSDEQLKSGTASVVCLLNNFYPREAKVQW |
| amino | KVDNALQSGNSQESVTEQDSKDSTYSLSSTLTLSKADYEKHKVYACEV |
| acid | THQGLSSPVTKSFNRGEC (SEQ ID NO: 22) |
| sequence: | |

The Fc domain of the heavy chain of this antibody comprises non-naturally occurring amino acids 234A, 235Q, 322Q, 252Y, 254T and 256E as numbered by the EU index as set forth in Kabat. These six non-naturally occurring amino acids are shown in bold underlined text in the sequence above.

mAb1 was determined not to induce significant degranulation of FcgRI-expressing human mast cells (as shown by % release of beta-hexosaminidase from human mast cells in culture) compared to a corresponding antibody with a wild-type (unmutated) IgG1 Fc domain (“mAbc”). Release of beta-hexosaminidase from human mast cells in culture (in presence of IFN gamma) was reduced by more than 50% with mAb1 compared to mAbc. Additionally, mAb1 did not show significant Fc receptor-dependent KIT agonist activity (as determined by KIT phosphorylation) compared to mAbc, even when cross-linked on THP-1 cells. Fc receptor-dependent KIT agonist activity (as determined by KIT phosphorylation with Fc receptors crosslinked) was reduced by more than 50% with mAb1 compared to mAbc.

### 6.2 Example 2

Healthy volunteers were given a single infusion of mAb1 at 0.3, 1, 3, or 9 mg/kg or placebo. Total plasma tryptase levels were measured using an Immunocap® assay that detects both the alpha and beta forms of tryptase. For each cohort, tryptase values were normalized to 100% for pre-treatment values and to 0% for the lower limit of quantitation of the assay (1 ng/mL). Mean values and standard error of the mean were plotted by dose.

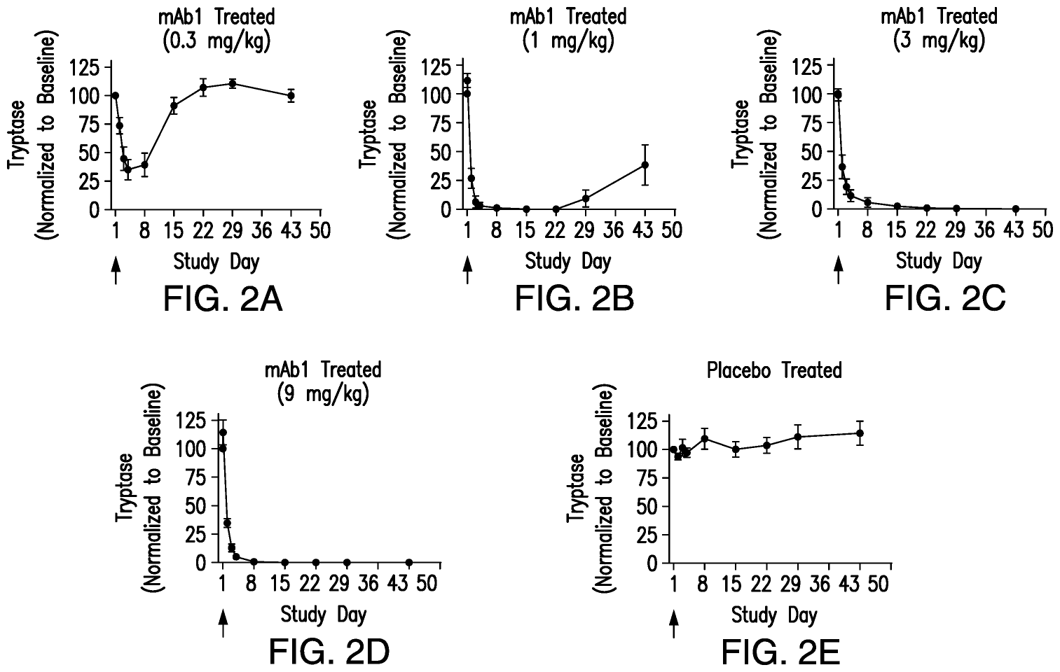

Results are shown in FIGS. 2A-2E, which indicates that mAb1 suppressed plasma tryptase in a dose-dependent manner.

### 6.3 Example 3

A single dose of mAb1 resulted in a prolonged decrease in tryptase values below the level of assay quantitation (1 ng/mL). Values below detection level were plotted arbitrarily as 0.5 ng/mL. Absolute plasma tryptase values were shown.

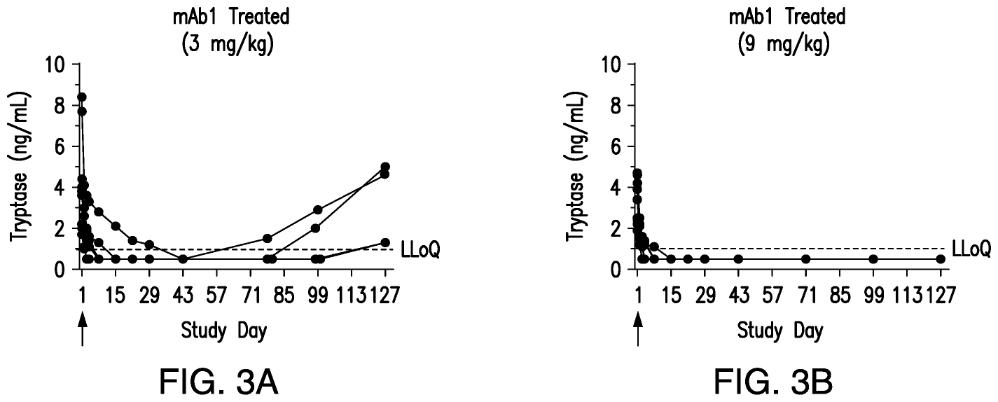

Results are shown in FIGS. 3A and 3B, which indicates that a single dose of mAb1 provided durable tryptase suppression at both 3 mg/kg and 9 mg/kg.

### 6.4 Example 4

Healthy volunteers were given a single infusion of mAb1 at 0.3, 1, 3, or 9 mg/kg or placebo. Plasma levels of stem cell factor (SCF), the only ligand for the c-KIT/CD117 receptor, were measured with an assay developed in house using a Meso Scale Diagnostics (MSD) platform. SCF plasma levels increased in a dose-dependent manner, which was consistent with allosteric blockade of SCF to the KIT receptor. Mean values and standard error of the mean were plotted by dose.

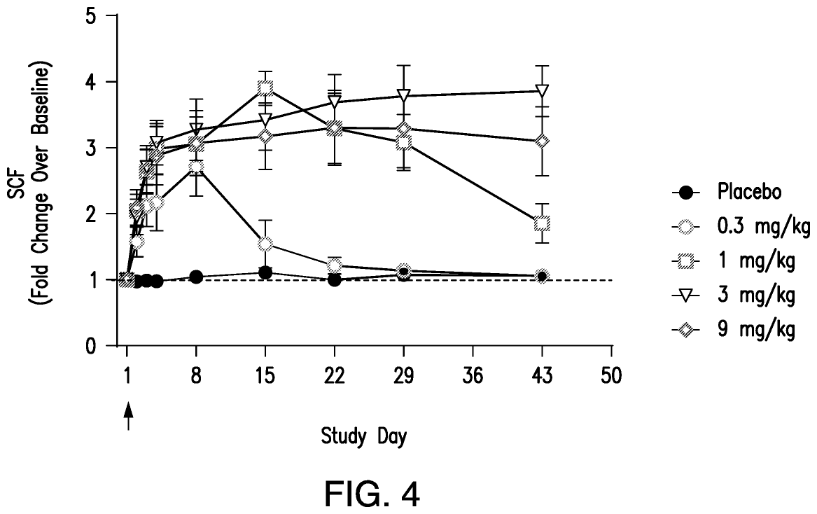

Results are shown in FIG. 4, which indicates that mAb1 induced a dose-dependent increase in plasma SCF levels.

### 6.5 Example 5

M-07e cells were serum-starved, then pre-treated with i) mAb1, ii) a corresponding antibody with the same variable region sequences but an unmutated (wild type) human IgG1 sequence (“mAbc”), iii) an isotype control antibody or iv) a small molecule kinase inhibitor that targets KIT (imatinib), and then stimulated with Stem Cell Factor (SCF). Phosphorylation was assessed by Western blotting.

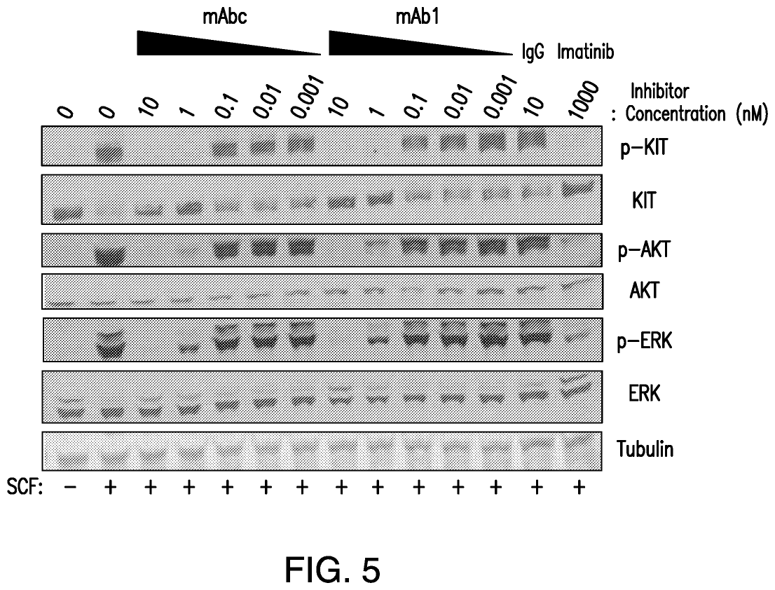

Results from one experiment are shown in FIG. 5. Data shown are representative of 3 independent experiments. IgG=human IgG1 isotype control antibody; p-KIT=tyrosine phosphorylated KIT; p-AKT=phosphorylated AKT; p-ERK=phosphorylated ERK1/2. Collectively, these results indicate that mAb1 was a more potent inhibitor of SCF-induced activation of wild-type KIT and downstream intracellular signaling pathways than the small molecule KIT inhibitor tested.

### 6.6 Example 6

The effects of mAb1 and imatinib on SCF-dependent M-07e cell proliferation were also characterized.

M-07e cells were serum-starved, then pre-treated with i) mAb1, ii) a corresponding antibody with the same variable region sequences but an unmutated (wild type) human IgG1 sequence (“mAbc”) or iii) imatinib, and then stimulated with SCF. Cells were incubated for 6 days at 37° C.

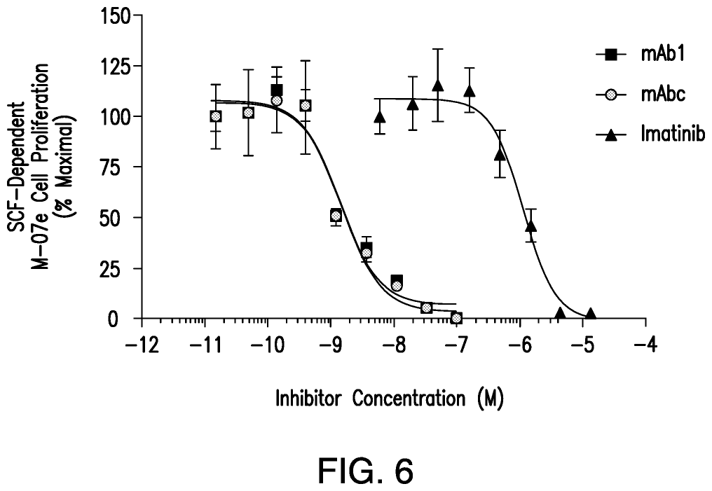

Results are shown in FIG. 6. Data are representative of 3 independent experiments and are presented as means of technical triplicates and standard errors of the means.

Both mAb1 and mAbc showed similar dose-dependent inhibition of M-07e cell proliferation, having mean IC50 values of 1.11±0.15 nM and 1.12±0.22 nM (±SEM; n=5; FIG. 6), respectively. By comparison, imatinib inhibited SCF-dependent M-07e cell proliferation less potently, with a mean IC50 of 228±39 nM (±SEM) from 3 independent experiments. The antagonist activity of mAb1 was comparable to mAbc, indicating that the mutations introduced into the Fc region of mAb1 did not affect its ability to inhibit KIT.

### 6.7 Example 7

The binding affinities of mAb1 for recombinant human Fc-gamma receptors (FcγRs) and human neonatal Fc Receptor (FcRn) were characterized and equilibrium KD values were generated.

Binding to recombinant human Fc receptors was measured by bio-layer interferometry using an Octet® QKe instrument. Histidine-tagged Fc receptors were captured on anti-Penta-His biosensors and then exposed to serial dilutions of mAbc or mAb1 for 2-3 minutes, followed by a dissociation step ranging from 5-10 minutes. Curve fitting was performed using the instrument's analysis software. Binding to FcRn was performed with association and dissociation steps at pH 6.0 as well as pH 7.2.

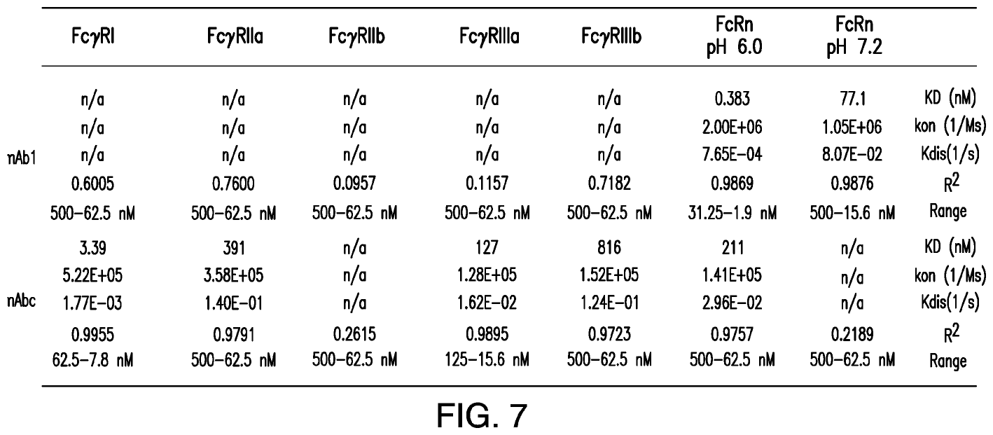

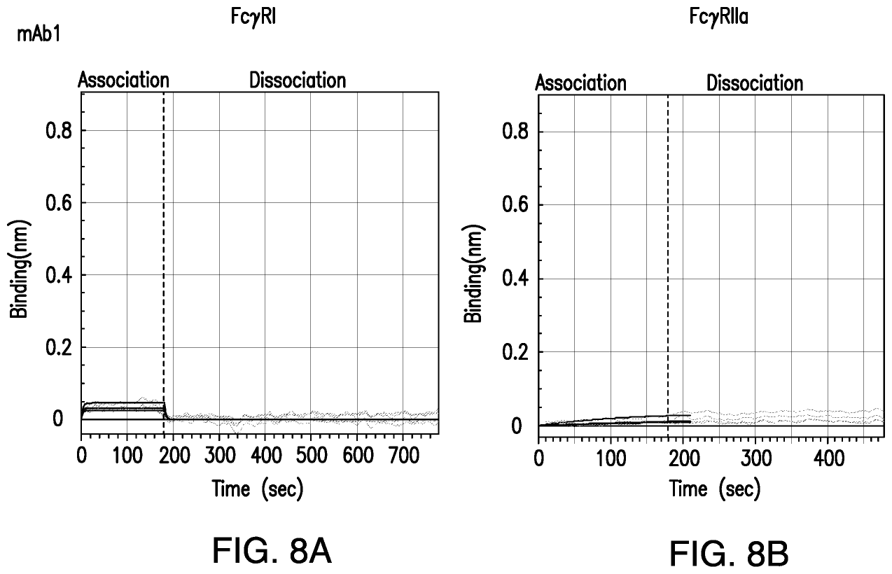

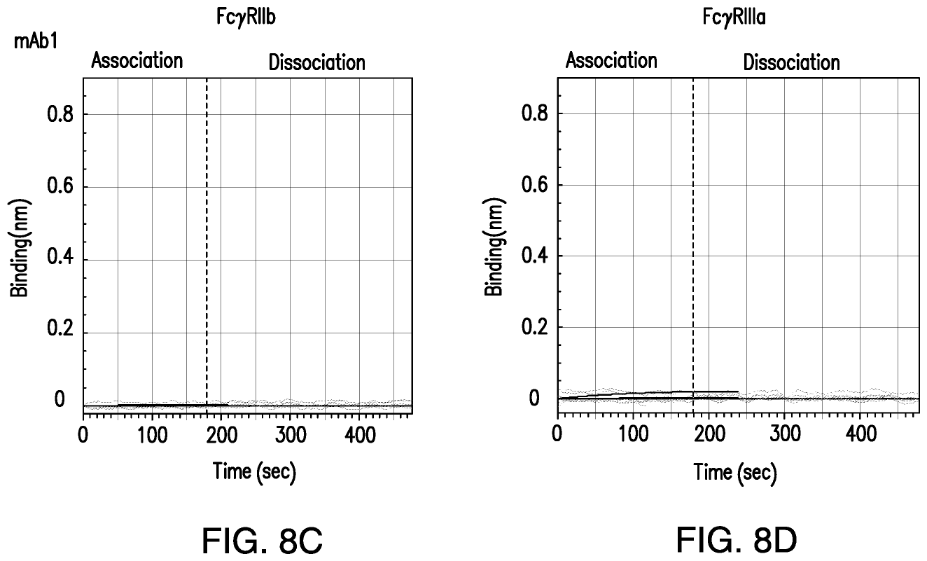

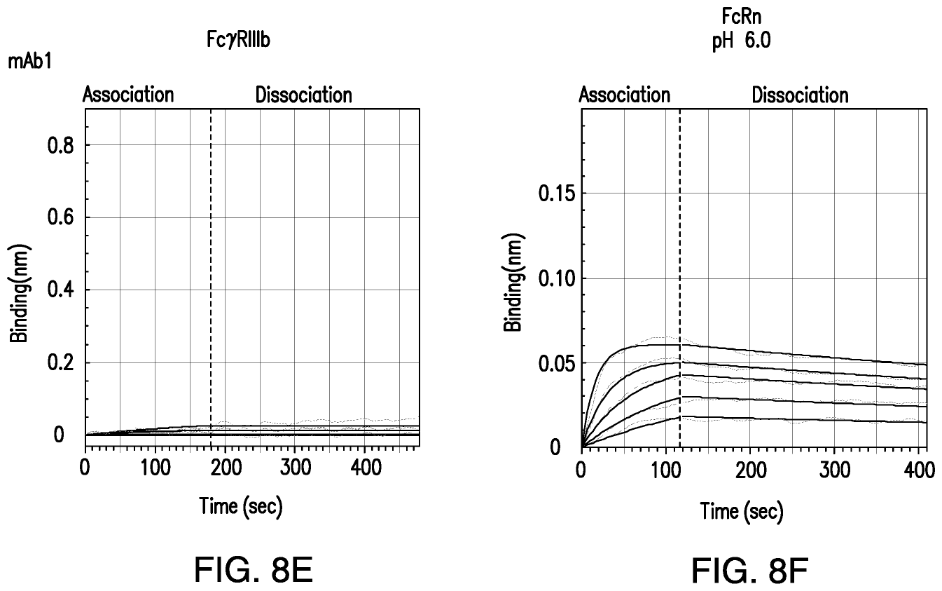

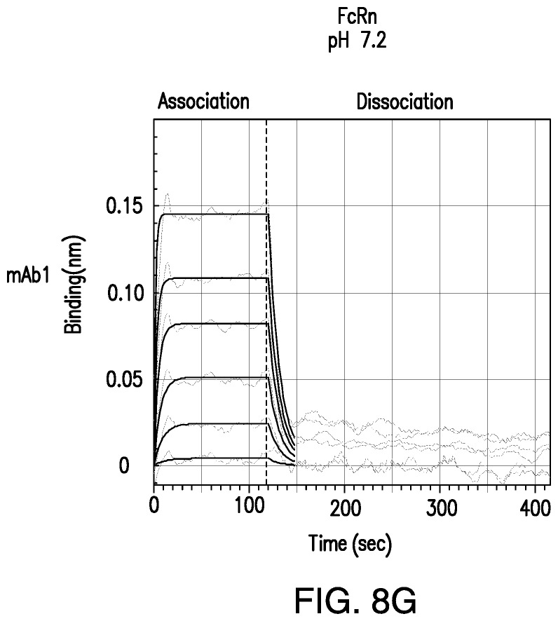

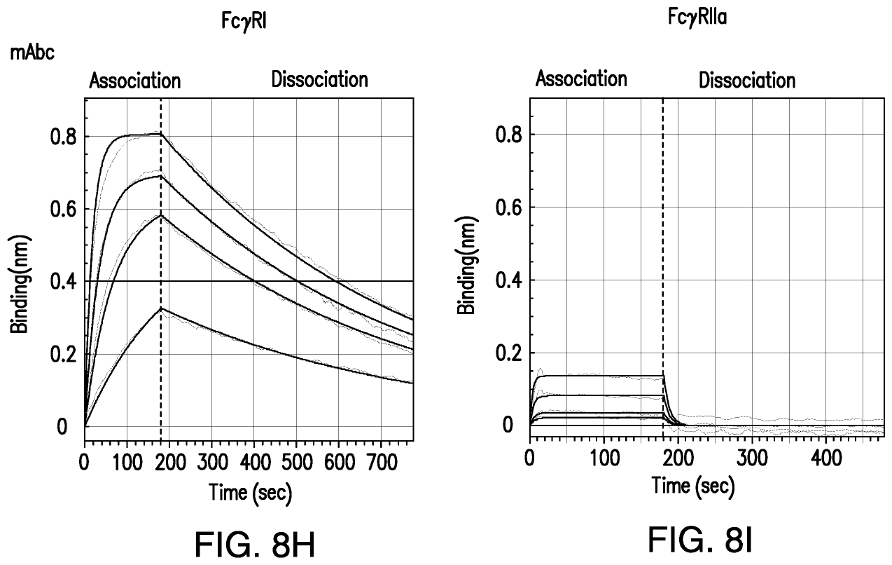

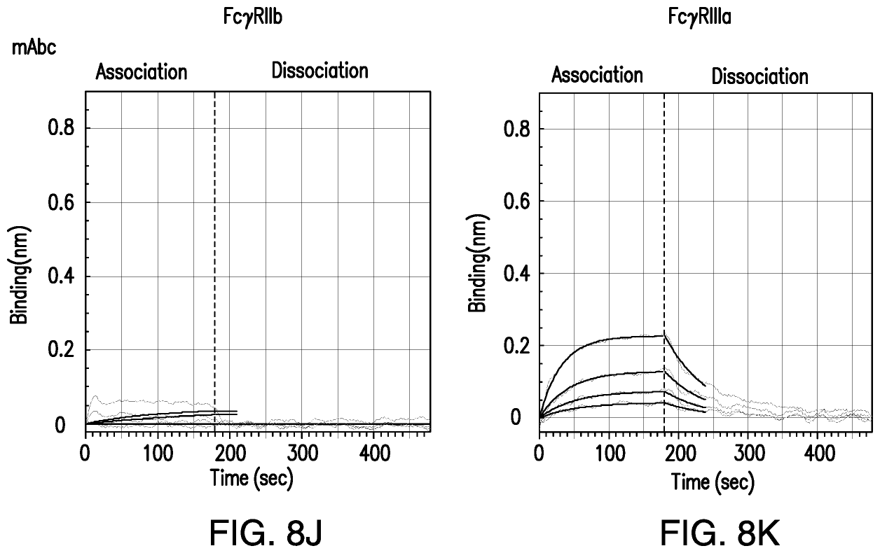

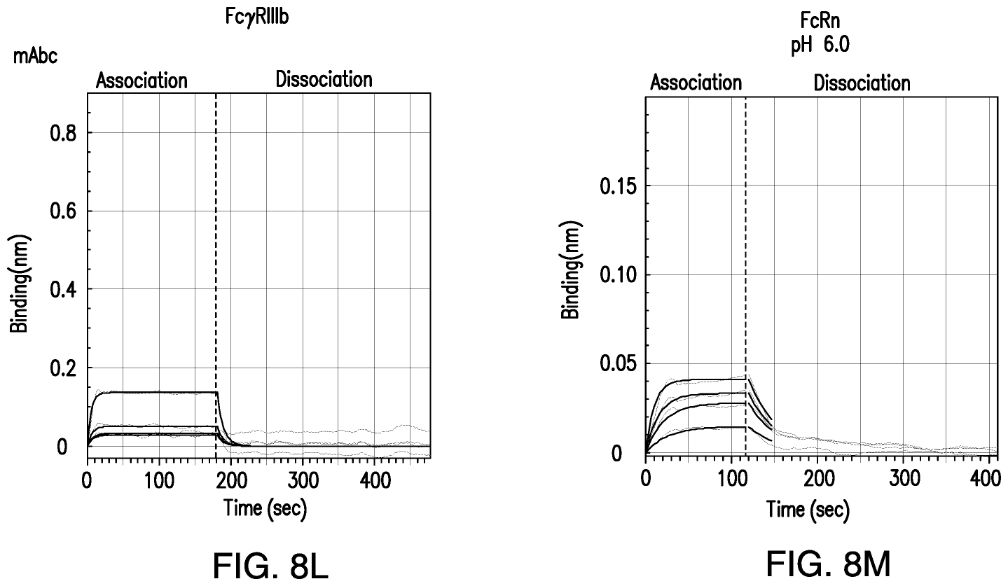

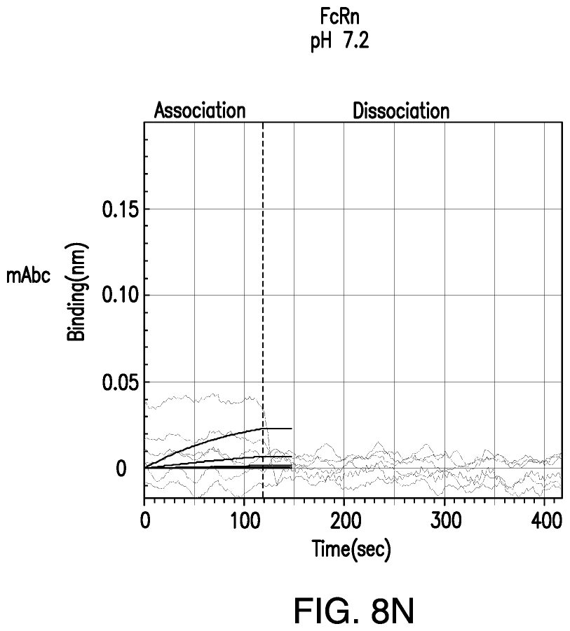

Results are shown in FIGS. 7 and 8A-8N, which show that mAbc bound with high affinity to FcγRI (KD=3.39 nM), with intermediate affinity to FcγRIIIa (KD=127 nM) and weak affinity to FcγRIIa (KD=391 nM) and FcγRIIIb (KD=816 nM). No binding to FcγRIIb was observed. In contrast, no binding of mAb1 (concentration=500 nM) to any recombinant human FcγRs was detected.

Binding to the human neonatal Fc Receptor (FcRn) was tested at physiological pH 7.2 as well as at pH 6.0 to simulate endocytic conditions. At pH 7.2, binding of mAbc to FcRn was not observed whereas mAb1 bound with intermediate affinity (KD=77.1 nM). At pH 6.0, mAbc bound to FcRn with intermediate affinity (KD=211 nM). However, mAb1 bound to FcRn at pH 6.0 with significantly higher affinity and showed very slow dissociation (KD=0.38 nM).

These data indicate that the Fc mutations in mAb1 abolished FcγR interactions while enhancing interactions with FcRn in vitro.

### 6.8 Example 8

Antibody-dependent cellular cytotoxicity (ADCC) activity of mAb1 was evaluated using a commercially available ADCC Reporter Bioassay Kit (Promega Corporation, Madison, WI). The ADCC Reporter Bioassay used engineered Jurkat cells that stably expressed the FcγRIIIa receptor V158 (high affinity) variant, and an nuclear factor of activated T cells (NFAT) response element driving expression of firefly luciferase as effector cells. Binding of FcγRIIIa receptors on the surface of effector cells to the Fc effector portion of antibodies bound to antigen on target cells would result in crosslinking and activation of FcγRIIIa signaling, which would induce expression of the NFAT reporter gene. To assess the ability of mAb1 and mAbc to induce ADCC, KIT-expressing M-07e cells, transfected CHO cells (CHO-WT KIT), and human small cell lung cancer H526 cells were used as target cells. In addition, untransfected CHO cells, which did not express KIT, were used as control target cells. ADCC was considered to have been activated if a dose-dependent increase in the resulting reporter gene signal was observed.

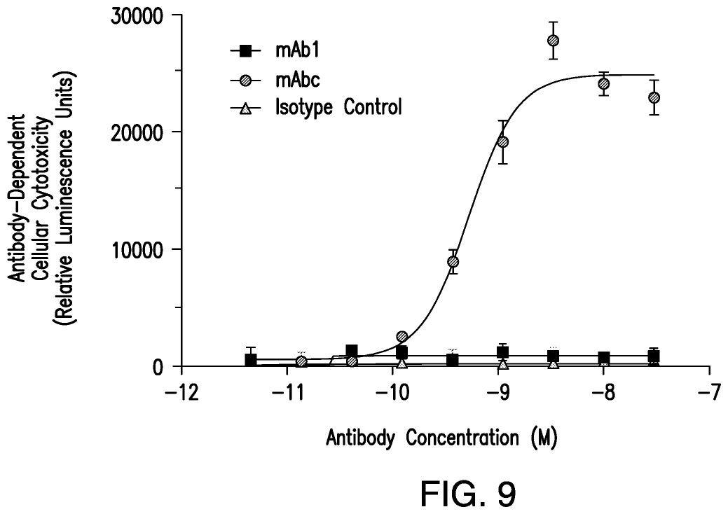

As shown in FIG. 9, mAbc triggered an ADCC response against KIT-expressing M-07e target cells, whereas mAb1 or the isotype control did not. Similar results were observed with CHO-WT KIT and H526 cells. Together, these data demonstrate that, unlike mAbc, mAb1 did not trigger ADCC responses against the KIT-expressing target cells examined.

### 6.9 Example 9

Fresh whole blood was incubated overnight with either 40 nM huIgG1 isotype control or mAb1 at 0.02, 0.2, or 40 nM, either in solution or dry coated as indicated. Phytohemagglutinin (PHA) (10 pg/mL) and lipopolysaccharides (LPS) (10 pg/mL) were used as positive controls. Plasma samples were harvested and stored frozen at −80° C. Cytokine levels were determined by utilizing Multiplexing Laser Bead Technology performed by Eve Technologies (Calgary, Alberta, Canada).

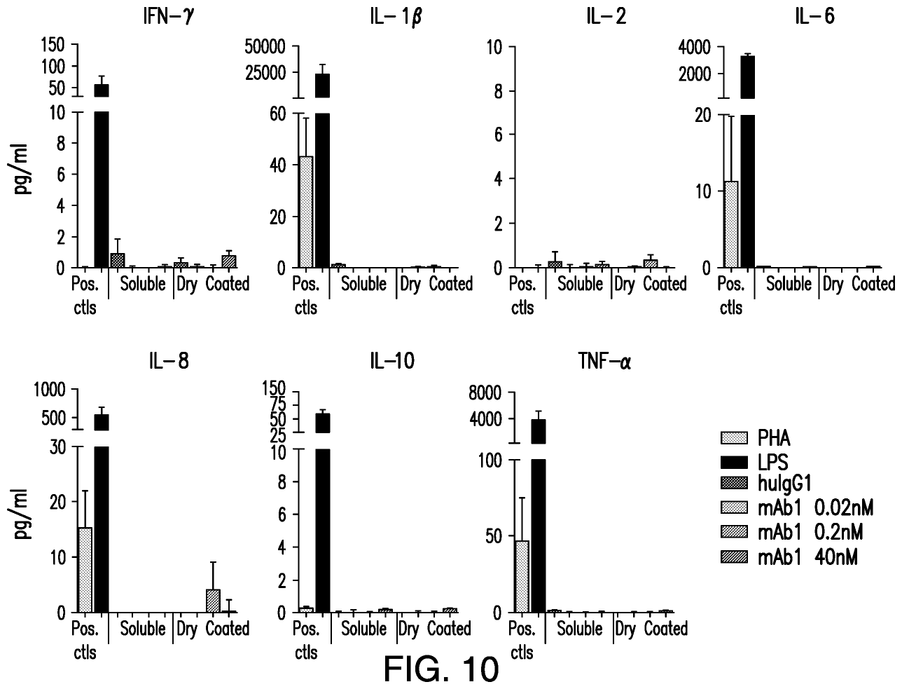

Results are shown in FIG. 10 in which cytokine concentrations for individual donors represent average of duplicate samples. Mean cytokine concentrations for all donors (n=6) were plotted with error bars representing SEM. The results show that overall very little specific cytokine induction was observed with mAb1 relative to the human IgG1 isotype control.

### 6.10 Example 10

A human patient presents with chronic pruritus and multiple pruriginous skin lesions. The patient is diagnosed with chronic prurigo and is administered mAb1 intravenously. The patient is monitored before, during, and after the treatment for response by clinical assessments.

### 6.11 Example 11

Described herein is a protocol for an open label, Phase 1 single dose study to assess the safety, pharmacokinetics, and phannacodynamics of mAb1 as add-on therapy in patients with cold contact urticaria, symptomatic dermographism, or cholinergic urticaria. The study type is interventional (clinical trial). The intervention mode is single group assignment.

This study is an open label Phase 1 study evaluating the safety, phannacokinetics, and pharmacodynamics of a single dose of mAb1 in patients with cold contact urticaria, symptomatic dermographism, or cholinergic urticaria who remain symptomatic despite treatment with antihistamines. It is estimated that ten patients with cold contact urticaria, ten patients with symptomatic dermographism, and ten patients with cholinergic urticaria will be enrolled in three separate cohorts for a total of 30 patients. Prospective patients are screened with tests in clinic as well as daily at home diaries for 2 weeks prior to enrollment. A single dose of mAb1 is administered intravenously on Day 1. Post-treatment, patients are followed for 12 weeks. Adult patients aged 18 years to 75 years of all sexes are eligible for the study. Healthy volunteers are not accepted.

Primary Outcome Measures:

1. Safety as assessed by the incidence and severity of adverse events (time frame: from Day 1 through week 12). Safety of a single dose of mAb1 as determined by adverse events.

Secondary Outcome Measures:

1. For patients with cold contact urticaria, change in critical temperature thresholds (CTT) (time frame: from Day 1 to Day 85). The change from baseline in critical temperature thresholds over time as determined by provocation testing using the TempTest®.

2. For patients with symptomatic dermographism, change in provocation thresholds (time frame: from Day 1 to Day 85). The change from baseline in provocation thresholds over time as determined by provocation testing using the FricTest®.

3. For patients with cholinergic urticaria, changes in baseline Urticaria Activity Score Provocation (UASprovo) (time frame: From Day 1 to Day 85). Changes from baseline and percentage of responders as measured by UASprovo.

4. Changes from baseline in Urticaria Control Test (UCT) (time frame: from Day 1 to Day 85). Changes from baseline and percentage of responders for the UCT and modified UCT.

5. Blood biomarkers (time frame: from Day 1 to Day 85). Pre-treatment and post treatment blood samples are collected and analyzed for changes in Stem Cell Factor.

6. Blood biomarkers (time frame: from Day 1 to Day 85). Pre-treatment and post treatment blood samples are collected and analyzed for changes in tryptase.

7. Pharmacokinetic evaluation (time frame: from Day 1 to Day 85). mAb1 concentrations are measured.

8. Immunogenicity evaluation (time frame: from Day 1 to Day 85). Patients are monitored for the development of anti-drug antibodies.

Key Inclusion Criteria:

1. Diagnosis of cold contact urticaria, symptomatic dermographism, or cholinergic urticaria which does not respond to antihistamines. Diagnosis for ≥3 months; symptoms of both hive (wheal) and itch/burning/painful sensation despite concurrent use of anti-histamines. During screening, in clinic, for cold contact urticaria, patients must have a positive cold stimulation test, for symptomatic dermographism, patients must have a positive FricTest®; and for cholinergic urticaria, patients must have a positive pulse-controlled ergometry (PCE) provocation test. Subject is on stable dose of antihistamines.

2. Other than a diagnosis of cold contact urticaria, symptomatic dermographism, or cholinergic urticaria, no other conditions which would introduce additional risk factors or would interfere with the study procedures, as determined by the investigator, based on a medical evaluation.

3. Female and male patients must use highly effective contraception from the time of the screening visit and for at least 150 days after receipt of study treatment.

4. Willing and able to comply with all study requirements and procedures including completion of a daily medication diary and questionnaires.

Key Exclusion Criteria:

1. A clearly defined diagnosis of hives or angioedema other than chronic urticaria.

2. Receipt of prior biologic therapy (e.g., omalizumab, dupilumab, ligelizumab) within past 3 months.

3. Treatment with immunosuppressives (e.g., systemic corticosteroids, cyclosporine, methotrexate, dapsone, cyclophosphamide, tacrolimus and mycophenolate mofetil, hydroxychloroquine or others) within 4 weeks or 5 half-lives.

4. Active COVID-19 infection.

5. HIV, hepatitis B or hepatitis C infection.

There are additional criteria that the treating doctor reviews with a candidate to confirm eligibility for the study.

### 6.12 Example 12

### 6.12.1 Study Background and Summary

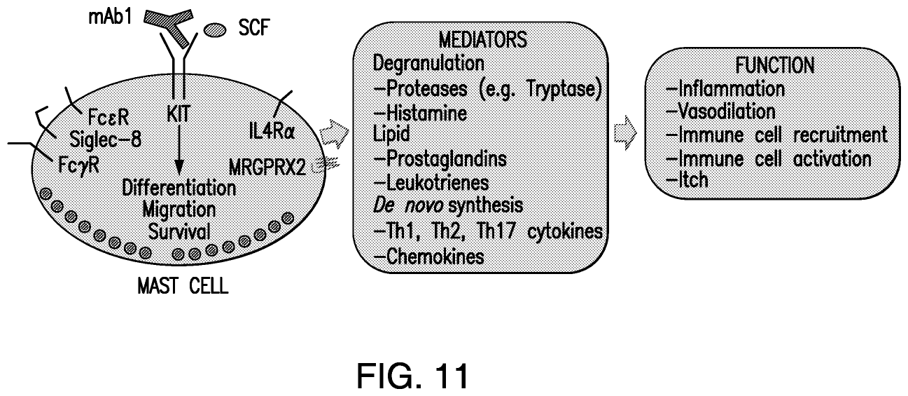

Chronic inducible urticaria (CIndU) is characterized by mast cell (MC)-driven wheals in response to triggers such as cold in cold urticaria (ColdU) or scratching of the skin in symptomatic dermographism (SD). These diseases, which are often severe and debilitating, can significantly impact patients' lives. MCs require activation of their KIT receptors by stem cell factor for survival, proliferation, and differentiation. MC burden is correlated with circulating tryptase, a protease secreted specifically by MCs. mAb1 is a monoclonal anti-KIT antibody that was engineered to selectively inhibit stem cell factor (SCF)-dependent KIT activation (see FIG. 11). mAb1 demonstrated a profound dose related reduction of circulating tryptase and was overall well-tolerated in healthy volunteers. In this study, patients with CIndU benefited from treatment with mAb1. One patient with SD enrolled in this study also had a diagnosis of prurigo nodularis, which improved after treatment with mAb1.

### 6.12.2 Study Design and Methods

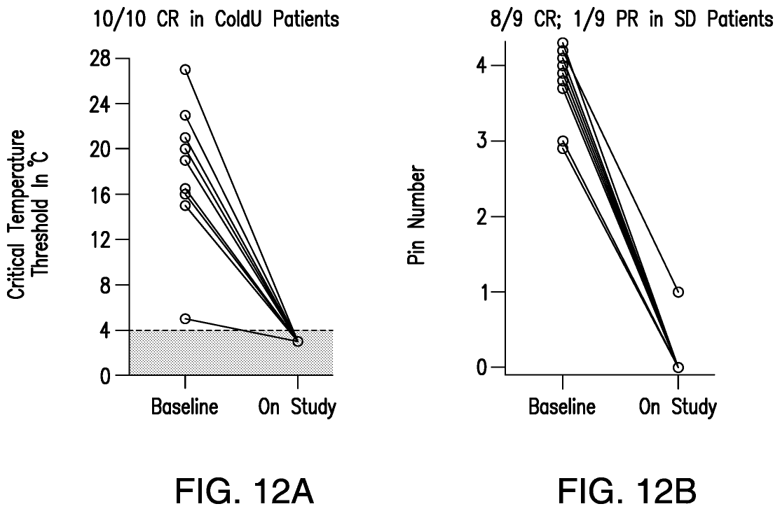

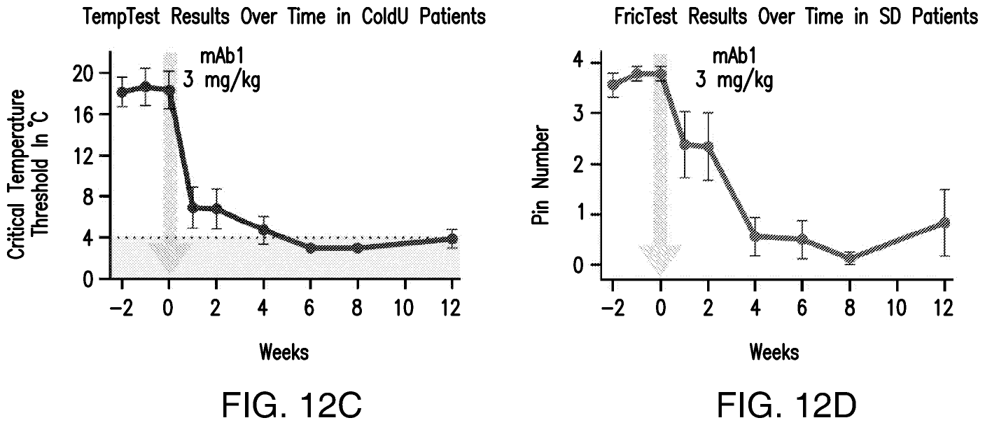

In this ongoing open-label, Phase 1b trial, patients with ColdU and SD refractory to antihistamine treatment received a single IV infusion of mAb1 at 3 mg/kg (as add-on treatment to H1-antihistamines) with a 12-week follow-up. Patients' ColdU and SD symptoms were induced via provocation testing that resembled real life triggering situations. Primary objective was to evaluate safety/tolerability of mAb1 (adverse events and clinical lab tests). Secondary and exploratory objectives included pharmacokinetic and pharmacodynamic assessments, including changes from baseline provocation thresholds, measurement of tryptase and stem cell factor levels, clinical activity outcomes (impact on urticaria symptoms, disease control, clinical response), quality of life assessments and measurement of tissue mast cells through skin biopsies. Secondary objectives included evaluating the effect of mAb1 on clinical activity and serum tryptase. Activity endpoints included provocation test (TempTest®/ColdU; FricTest®/SD), physician's global assessment (Phys-GA), and patient's global assessment (Pat-GA) of disease severity. Mean±standard error (SE) are shown in FIGS. 12C-12D, FIGS. 14A-14C, FIGS. 15A-15D and FIGS. 16A-16D for provocation tests, biomarkers and hematology, respectively. Skin MC numbers assessed using non-lesional skin biopsies were enumerated by tryptase staining.

One patient with SD enrolled in this study also had a diagnosis of prurigo nodularis.

The study was amended to add a cohort of patients with cholinergic urticaria.

The study was performed essentially according to the clinical trial protocol described in Example 11.

### 6.12.3 Study Status

Twenty patients received study drug (i.e., mAb1) by a single intravenous infusion at 3 mg/kg and were included in the safety analysis. Eleven had ColdU and 9 patients had SD. One of the 9 SD patients also had prurigo nodularis. Patients had high disease activity of CIndU as assessed by provocation threshold testing. In patients with ColdU, baseline critical temperature thresholds were 18.9° C./66° F. (range: 5-27° C./41-80.6° F.). In patients with SD, baseline FricTest® thresholds were 3.8 (range: 3-4) of 4 pins.

Nineteen patients received full dose and were included in the activity analysis, including the patient who had both SD and prurigo nodularis. Fourteen of 19 patients completed the 12-week observation period, including the patient who had both SD and prurigo nodularis; 5 were ongoing.

### 6.12.4 Demographics and Baseline Disease Characteristics

See Table 8 below for characterization of the 20 patients. All of the patients had prior antihistamine treatment.

TABLE 8
Demographics and baseline disease characteristics.

| | | ColdU (N = 11) | | SD (N = 9) | | All (N = 20) | |
|---|---|---|---|---|---|---|---|
| Age median (range) years | | 43 | (27-65) | 41 | (27-56) | 42 | (27-65) |
| Gender Female, n (%) | | 6 | (54.5%) | 4 | (44.4%) | 10 | (50%) |
| Race | White, n (%) | 10 | (90.9%) | 9 | (100%) | 19 | (95%) |
| | Asian, n (%) | 1 | (9.1%) | 0 | (0%) | 1 | (5%) |
| Ethnicity Hispanic or Latino | | 1 | (9.1%) | 0 | (0%) | 1 | (5%) |
| Weight median (range) kg | | 77.0 | (61.0-93.0) | 88.2 | (57.0-122.0) | 80.0 | (57.0-122.0) |
| Disease Duration | <5 yr, n (%) | 6 | (54.5%) | 4 | (44.4%) | 10 | |
| | ≥5 yr, n (%) | 5 | (45.5%) | 5 | (55.6%) | 10 | |
| History of Angioedema | | 6 | (54.5%) | 0 | | 6 | (30%) |
| Provocation Threshold Mean (range) | | 18.9 | (5.0-27.0) ° C. | 3.8 | (3-4) pins | | |
| Phys-GA ≥2, n (%) | | 10 | (91%) | 9 | (100%) | 19 | (95%) |
| Pat-GA ≥2, n (%) | | 6 | (55%) | 8 | (89%) | 14 | (70%) |
| Prior Medication | H1 Antihistamine | 11 | (100%) | 9 | (100%) | 20 | (100%) |
| | Biologics (omalizumab) | 1 | (9%) | 2 | (22%) | 3 | (15%) |

| | | | | | | |
|---|---|---|---|---|---|---|
| Tryptase median (range) ng/ml | 3.6 | (2.1-5.5) | 4.7 | (1.6-8.6) | 4.1 | (1.6-8.6) |

### 6.12.5 Study Results

As shown in FIGS. 12A-12D, a single dose of mAb1 (3 mg/kg) resulted in a rapid, profound, and durable response in patients with CIndU refractory to antihistamines. Complete response (CR) was achieved in 95% (18/19) patients (100% (10/10) in ColdU (FIG. 12A) and 89% (8/9) in SD (FIG. 12B) patients). Complete response was observed in all 3 patients (1 ColdU patient and 2 SD patients) with prior Xolair® (omalizumab) experience, including two who were Xolair® refractory. Rapid onset of response after dosing and sustained durability were observed. Most patients with ColdU and SD experienced a complete response by week 1 and by week 4, respectively. The CR was sustained for a median duration of 77 days in ColdU and 57 days in SD patients who completed the 12 week follow-up period (i.e., 8 ColdU patients and 6 SD patients) (FIGS. 12C and 12D). One of the 19 patients (an SD patient) experienced a partial response (PR). CR=negative provocation test at ≤4° C. (for ColdU) or 0 pins (for SD). PR=improvement by 4° C. (for ColdU) or ≥2 pins (for SD).

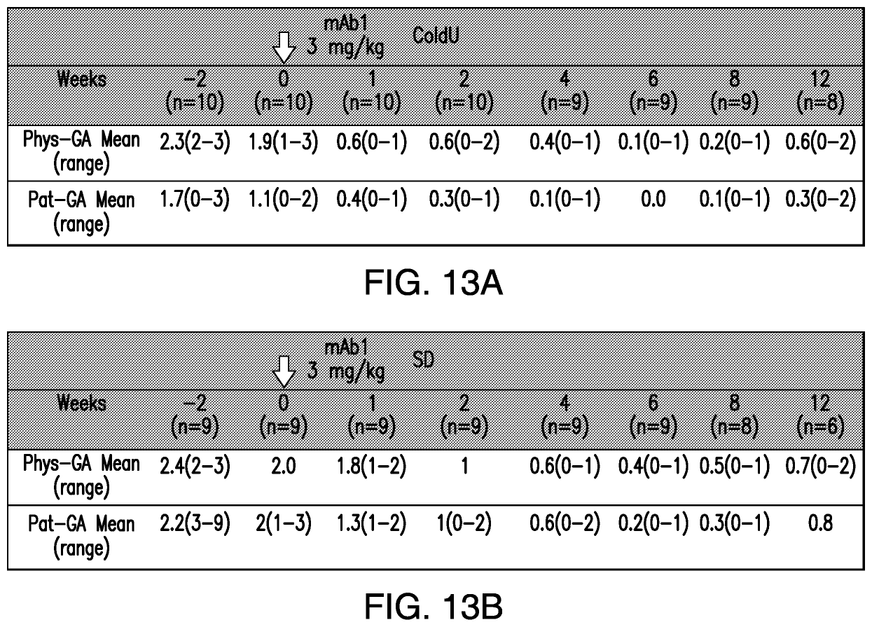

Improved disease activity as assessed by Phys-GA and Pat-GA was consistent with the complete response as measured by the provocation test (FIGS. 13A and 13B, for ColdU and SD patients, respectively).

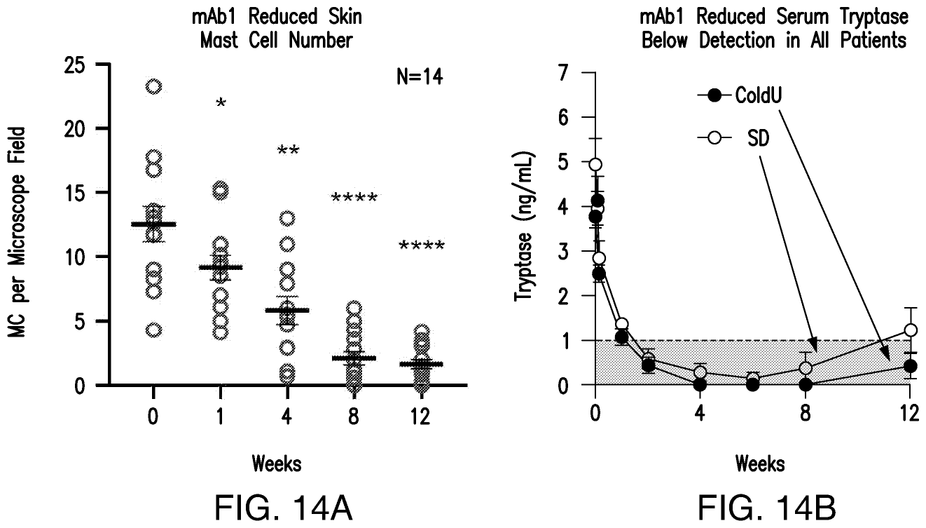

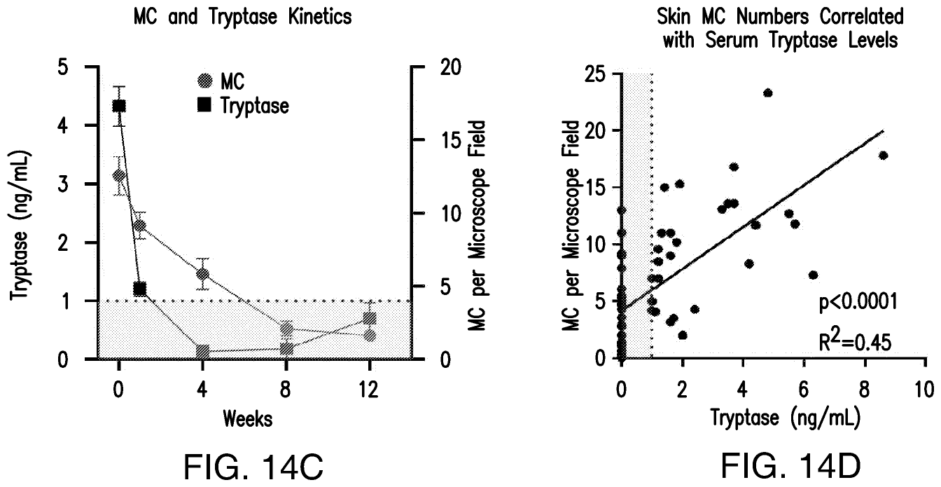

As shown in FIGS. 14A and 14B, a single 3 mg/kg mAb1 dose resulted in a rapid, marked, and durable depletion of skin MCs (87% depletion, see FIG. 14A) and suppression of serum tryptase (FIG. 14B) as measured through biopsy. The MC and tryptase kinetics exhibited similar trends over time (FIG. 14C). In addition, skin MC numbers positively correlated with serum tryptase levels (FIG. 14D).

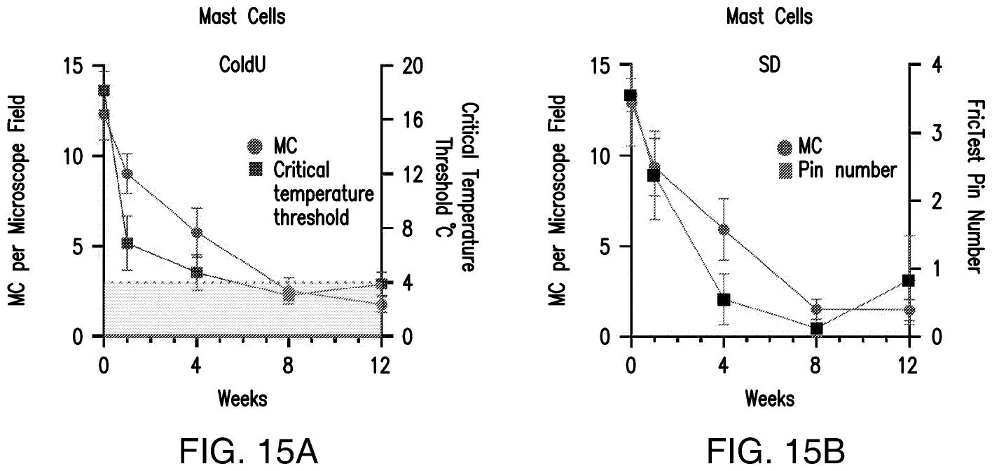

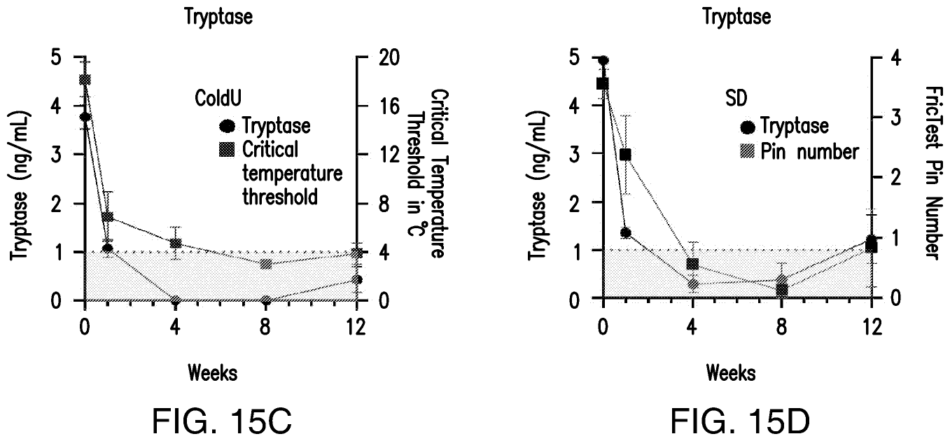

The kinetics of skin MC and serum tryptase depletion mirrored clinical activity of CIndU. In particular, the kinetics of skin MC and serum tryptase depletion mirrored decreases in provocation thresholds (FIGS. 15A-15D). The data confirmed that serum tryptase level is a robust pharmacodynamic biomarker for assessing MC burden and clinical activity in patients with CIndU and potentially in other diseases with mast cell driven involvement.

The patient who had both SD and prurigo nodularis experienced both a complete response of SD and notable improvement of the prurigo nodularis after a single dose of mAb1.

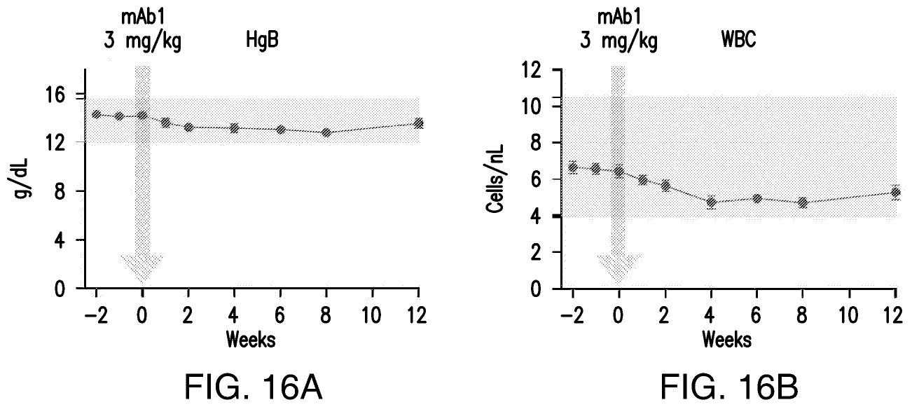

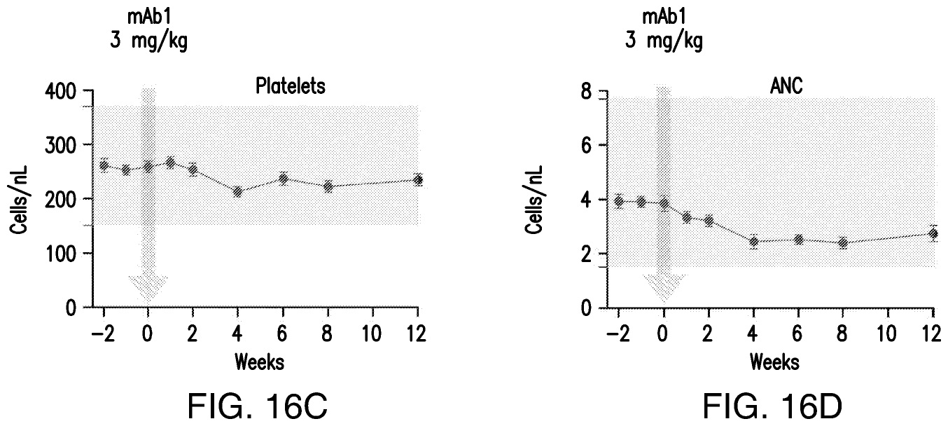

In addition, mAb1 demonstrated favorable safety and tolerability. mAb1 was generally well tolerated in the patients treated. The most common adverse events were hair color changes (14/20 (70%)), infusion reactions (9/20 (45%)), and taste disorders (8/20 (40%)). Hair color changes (generally small areas of hair color lightening) and taste disorders (generally partial changes of ability to taste salt) were consistent with inhibiting KIT signaling in other cell types and were expected to be fully reversible. Most adverse events were mild. Hair color changes improved upon longer observation period. Infusion reactions, generally manifested as hives and itching, resolved spontaneously. A single severe infusion reaction of brief loss of consciousness occurred in a patient with a history of fainting and was not attributed to MC activation as measured by serum tryptase monitoring. The patient rapidly recovered. Taste disorders were selective and transient. Hematology parameters generally remained within the normal ranges (see FIGS. 16A-16D). Mild, transient, and asymptomatic decreases in hemoglobin and white blood cell (WBC) parameters were noted (see FIGS. 16A-16D). However, there was no evidence of clinically significant decreases in hematology parameters. This was an important finding for a KIT inhibitor.

Together, the data show that mAb1 demonstrated unprecedented MC depletion with a favorable safety profile, providing a significant potential as a therapy for CIndU for quick, lasting, and meaningful relief, indicating its potential to impact other diseases with mast cell involvement, and opening opportunities for the evaluation of MC involvement across many diseases.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed:

1. A method of protecting against, treating, or managing chronic prurigo in a subject, comprising administering to a subject in need thereof a therapeutically effective amount of an antibody which immunospecifically binds to human KIT, or an antigen binding fragment thereof.

2. The method of claim 1, wherein the human KIT comprises the amino acid sequence of SEQ ID NO: 1.

3. The method of claim 1 or 2, wherein the chronic prurigo is prurigo nodularis.

4. The method of any one of claims 1 to 3, wherein the antibody is a bivalent monospecific antibody.

5. The method of any one of claims 1 to 3, wherein the antibody is a bispecific antibody.

6. The method of any one of claims 1 to 5, wherein the antibody is a humanized antibody.

7. The method of any one of claims 1 to 6, wherein the antibody comprises a modified Fc region or domain.

8. The method of any one of claims 1 to 7, wherein the antibody has reduced Fc receptor binding activity.

9. The method of claim 8, wherein the antibody has reduced FcγR binding activity.

10. The method of any one of claims 1 to 9, wherein the antibody does not induce significant degranulation of FcgRI-expressing human mast cells.

11. The method of any one of claims 1 to 10, wherein the antibody does not show significant Fc receptor-dependent KIT agonist activity.

12. The method of any one of claims 1 to 11, wherein the antibody specifically binds to a D4 or D5 region of human KIT.

13. The method of any one of claims 1 to 12, wherein the antibody comprises:
(A) (i) a light chain variable region (“VL”) comprising VL CDR1, VL CDR2, and VL CDR3 comprising the amino acid sequences of SEQ ID NO: 2, SEQ ID NO: 3, and SEQ ID NO: 4, respectively; and
(ii) a heavy chain variable region (“VH”) comprising VH CDR1, VH CDR2, and VH CDR3 comprising the amino acid sequences of SEQ ID NO: 5, SEQ ID NO: 6, and SEQ ID NO: 7, respectively;
(B) (i) a VL comprising VL CDR1, VL CDR2, and VL CDR3 comprising the amino acid sequences of SEQ ID NO: 2, SEQ ID NO: 3, and SEQ ID NO: 4, respectively; and
(ii) a VH comprising VH CDR1, VH CDR2, and VH CDR3 comprising the amino acid sequences of SEQ ID NO: 25, SEQ ID NO: 26, and SEQ ID NO: 27, respectively;
(C) (i) a VL comprising VL CDR1, VL CDR2, and VL CDR3 comprising the amino acid sequences of SEQ ID NO: 28, SEQ ID NO: 29, and SEQ ID NO: 30, respectively; and
(ii) a VH comprising VH CDR1, VH CDR2, and VH CDR3 comprising the amino acid sequences of SEQ ID NO: 25, SEQ ID NO: 31, and SEQ ID NO: 32, respectively;
(D) (i) a VL comprising VL CDR1, VL CDR2, and VL CDR3 comprising the amino acid sequences of SEQ ID NO: 2, SEQ ID NO: 3, and SEQ ID NO: 4, respectively; and
(ii) a VH comprising VH CDR1, VH CDR2, and VH CDR3 comprising the amino acid sequences of SEQ ID NO: 33, SEQ ID NO: 34, and SEQ ID NO: 27, respectively; or
(E) (i) a VL comprising VL CDR1, VL CDR2, and VL CDR3 comprising the amino acid sequences of SEQ ID NO: 35, SEQ ID NO: 36, and SEQ ID NO: 37, respectively; and
(ii) a VH comprising VH CDR1, VH CDR2, and VH CDR3 comprising the amino acid sequences of SEQ ID NO: 38, SEQ ID NO: 39, and SEQ ID NO: 40, respectively.

14. The method of claim 13, wherein the antibody comprises a VL comprising VL CDRs 1-3 comprising the amino acid sequences of SEQ ID NOs: 2-4, respectively, and a VH comprising VH CDRs 1-3 comprising the amino acid sequences of SEQ ID NOs: 5-7, respectively.

15. The method of any one of claims 1 to 14, wherein the antibody comprises
(i) a VL comprising the amino acid sequence:
DIVMTQSPSX_{K1}LSASVGDRVTITCKASQNVRTNVAWYQQKPGKAPKX_{K2}LIYSASYRYS GVPDRFX_{K3}GSGSGTDFTLTISSLQX_{K4}EDFAX_{K5}YX_{K6}CQQYNSYPRTFGGGTKVEIK (SEQ ID NO: 17), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and
(ii) a VH comprising the amino acid sequence:
QVQLVQSGAEX_{H1}KKPGASVKX_{H2}SCKASGYTFTDYYINWVX_{H3}QAPGKGLEWIARIYPG SGNTYYNEKFKGRX_{H4}TX_{H5}TAX_{H6}KSTSTAYMX_{H7}LSSLRSEDX_{H8}AVYFCARGVYYFDY WGQGTTVTVSS (SEQ ID NO: 18), wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain.

16. The method of claim 15, wherein X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

17. The method of any one of claims 1 to 16, wherein the antibody comprises a VL comprising an amino acid sequence selected from the group consisting of SEQ ID NOs: 13, 14, 15, and 16; and a VH comprising an amino acid sequence selected from the group consisting of SEQ ID NOs: 8, 9, 10, 11, and 12.

18. The method of any one of claims 1 to 17, wherein the antibody comprises a human light chain constant region.

19. The method of any one of claims 1 to 18, wherein the antibody comprises a human heavy chain constant region.

20. The method of claim 19, wherein the human heavy chain constant region is a human IgG constant region.

21. The method of claim 20, wherein the human heavy chain constant region is a human IgG1 constant region.

22. The method of any one of claims 1 to 21, wherein the antibody comprises a modified human Fc region or domain.

23. The method of any one of claims 1 to 21, wherein the antibody comprises a modified human IgG1 Fc region or domain.

24. The method of claim 23, wherein the modified human IgG1 Fc region or domain comprises non-naturally occurring amino acids 234A, 235Q and 322Q as numbered by the EU index as set forth in Kabat.

25. The method of claim 24, wherein the modified human IgG1 Fc region or domain further comprises non-naturally occurring amino acids 252Y, 254T and 256E as numbered by the EU index as set forth in Kabat.

26. The method of any one of claims 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified human IgG1 Fc region or domain comprising non-naturally occurring amino acids 234A, 235Q and 322Q as numbered by the EU index as set forth in Kabat.

27. The method of any one of claims 1 to 25, wherein the antibody comprises:
(i) a VL comprising an amino acid sequence of SEQ ID NO: 14;
(ii) a VH comprising an amino acid sequence of SEQ ID NO: 10; and
(iii) a modified human IgG1 Fc region or domain comprising non-naturally occurring amino acids 234A, 235Q, 322Q, 252Y, 254T and 256E as numbered by the EU index as set forth in Kabat.

28. The method of any one of claims 1 to 25, wherein the antibody comprises a heavy chain comprising the amino acid sequence:
(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG.

29. The method of any one of claims 1 to 25, wherein the antibody comprises a light chain comprising the amino acid sequence:
(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

30. The method of any one of claims 1 to 25, wherein the antibody comprises a heavy chain comprising the amino acid sequence:
QVQLVQSGAEVKKPGASVKLSCKASGYTFTDYYINWVRQAPGKGLEWIARIYPGSGNT YYNEKFKGRATLTADKSTSTAYMQLSSLRSEDTAVYFCARGVYYFDYWGQGTTVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAQG GPSVFLFPPKPKDTLYITREPEVTCVVVDVSHEDPEVKFNWYVDGVEVHNAKTKPREEQ YNSTYRVVSVLTVLHQDWLNGKEYKCQVSNKALPAPIEKTISKAKGQPREPQVYTLPPS RDELTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 21); and a light chain comprising the amino acid sequence:
(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

31. The method of any one of claims 1 to 30, wherein the subject is a human adult.

32. The method of any one of claims 1 to 30, wherein the subject is a human child.


SEQUENCE LISTING (40 sequences)

SEQ ID NO: 1 - protein, 972 aa, Homo sapiens
  human KIT receptor
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 2 - protein, 11 aa, Artificial Sequence
  light chain CDR1 of anti-KIT antibody (VL-CDR1 - AbM)
KASQNVRTNVA

SEQ ID NO: 3 - protein, 7 aa, Artificial Sequence
  light chain CDR2 of anti-KIT antibody (VL-CDR2 - AbM)
SASYRYS

SEQ ID NO: 4 - protein, 9 aa, Artificial Sequence
  light chain CDR3 of anti-KIT antibody (VL-CDR3 - AbM)
QQYNSYPRT

SEQ ID NO: 5 - protein, 5 aa, Artificial Sequence
  heavy chain CDR1 of anti-KIT antibody (VH-CDR1)
DYYIN

SEQ ID NO: 6 - protein, 17 aa, Artificial Sequence
  heavy chain CDR2 of anti-KIT antibody (VH-CDR2)
RIYPGSGNTYYNEKFKG

SEQ ID NO: 7 - protein, 7 aa, Artificial Sequence
  heavy chain CDR3 of anti-KIT antibody (VH-CDR3)
GVYYFDY

SEQ ID NO: 8 - protein, 116 aa, Artificial Sequence
  heavy chain variable region of anti-KIT antibody (VH1)
QVQLVQSGAELKKPGASVKLSCKASGYTFTDYYINWVKQAPGKGLEWIARIYPGSGNTYYNEKFKGRATLTAEKSTSTAYMQLSSLRSEDSAVYFCARGVYYFDYWGQGTTVTVSS

SEQ ID NO: 9 - protein, 116 aa, Artificial Sequence
  heavy chain variable region of anti-KIT antibody (VH2)
QVQLVQSGAEVKKPGASVKLSCKASGYTFTDYYINWVKQAPGKGLEWIARIYPGSGNTYYNEKFKGRATLTAEKSTSTAYMQLSSLRSEDTAVYFCARGVYYFDYWGQGTTVTVSS

SEQ ID NO: 10 - protein, 116 aa, Artificial Sequence
  heavy chain variable region of anti-KIT antibody (VH3)
QVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSS

SEQ ID NO: 11 - protein, 116 aa, Artificial Sequence
  heavy chain variable region of anti-KIT antibody (VH4)
QVQLVQSGAEVKKPGASVKVSCKASGYTFTDYYINWVRQAPGKGLEWIARIYPGSGNTYYNEKFKGRATITADKSTSTAYMELSSLRSEDTAVYFCARGVYYFDYWGQGTTVTVSS

SEQ ID NO: 12 - protein, 116 aa, Artificial Sequence
  heavy chain variable region of anti-KIT antibody (VH5)
QVQLVQSGAEVKKPGASVKVSCKASGYTFTDYYINWVRQAPGKGLEWIARIYPGSGNTYYNEKFKGRVTITADKSTSTAYMELSSLRSEDTAVYFCARGVYYFDYWGQGTTVTVSS

SEQ ID NO: 13 - protein, 107 aa, Artificial Sequence
  light chain variable region of anti-KIT antibody (VL1)
DIVMTQSPSFLSASVGDRVTITCKASQNVRTNVAWYQQKPGKAPKALIYSASYRYSGVPDRFTGSGSGTDFTLTISSLQSEDFADYFCQQYNSYPRTFGGGTKVEIK

SEQ ID NO: 14 - protein, 107 aa, Artificial Sequence
  light chain variable region of anti-KIT antibody (VL2)
DIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIK

SEQ ID NO: 15 - protein, 107 aa, Artificial Sequence
  light chain variable region of anti-KIT antibody (VL3)
DIVMTQSPSSLSASVGDRVTITCKASQNVRTNVAWYQQKPGKAPKALIYSASYRYSGVPDRFSGSGSGTDFTLTISSLQPEDFADYFCQQYNSYPRTFGGGTKVEIK

SEQ ID NO: 16 - protein, 107 aa, Artificial Sequence
  light chain variable region of anti-KIT antibody (VL4)
DIVMTQSPSSLSASVGDRVTITCKASQNVRTNVAWYQQKPGKAPKSLIYSASYRYSGVPDRFSGSGSGTDFTLTISSLQPEDFATYYCQQYNSYPRTFGGGTKVEIK

SEQ ID NO: 17 - protein, 107 aa, Artificial Sequence
  light chain variable region consensus sequence
  misc_feature (10)..(10): Xaa can be any amino acid; or in specific enbodiment, it can be amino acid with an aromatic or aliphatic hydroxyl side chain; or in another specific embodiment, Xaa is the amino acid Phe or Ser
  misc_feature (46)..(46): Xaa can be any amino acid; or in specific enbodiment, it can be amino acid with an aliphatic or aliphatic hydroxyl side chain; or in another specific embodiment, Xaa is the amino acid Ala or Ser
  misc_feature (63)..(63): Xaa can be any amino acid; or in specific enbodiment, it can be amino acid with an aliphatic or aliphatic hydroxyl side chain; or in another specific embodiment, Xaa is the amino acid Thr or Ser
  misc_feature (80)..(80): Xaa can be any amino acid; or in specific enbodiment, it can be amino acid with an aliphatic or aliphatic hydroxyl side chain; or in another specific embodiment, Xaa is the amino acid Ser or Pro
  misc_feature (85)..(85): Xaa can be any amino acid; or in specific enbodiment, it can be amino acid with a charged or acidic side chain; or in another specific embodiment, Xaa is the amino acid Asp or Thr
  misc_feature (87)..(87): Xaa can be any amino acid; or in specific enbodiment, it can be amino acid with an aromatic side chain; or in another specific embodiment, Xaa is the amino acid Phe or Tyr
DIVMTQSPSXLSASVGDRVTITCKASQNVRTNVAWYQQKPGKAPKXLIYSASYRYSGVPDRFXGSGSGTDFTLTISSLQXEDFAXYXCQQYNSYPRTFGGGTKVEIK

SEQ ID NO: 18 - protein, 116 aa, Artificial Sequence
  heavy chain variable region consensus sequence
  misc_feature (11)..(11): Xaa can be any amino acid; or in specific enbodiment, it can be amino acid with an aliphatic side chain; or in another specific embodiment, Xaa is the amino acid Leu or Val
  misc_feature (20)..(20): Xaa can be any amino acid; or in specific enbodiment, it can be amino acid with an aliphatic side chain; or in another specific embodiment, Xaa is the amino acid Leu or Val
  misc_feature (38)..(38): Xaa can be any amino acid; or in specific enbodiment, it can be amino acid with a polar or basic side chain; or in another specific embodiment, Xaa is the amino acid Lys or Arg
  misc_feature (68)..(68): Xaa can be any amino acid; or in specific enbodiment, it can be amino acid with an aliphatic side chain; or in another specific embodiment, Xaa is the amino acid Val or Ala
  misc_feature (70)..(70): Xaa can be any amino acid; or in specific enbodiment, it can be amino acid with an aliphatic side chain; or in another specific embodiment, Xaa is the amino acid Leu or Ile
  misc_feature (73)..(73): Xaa can be any amino acid; or in specific enbodiment, it can be amino acid with an acidic side chain; or in another specific embodiment, Xaa is the amino acid Glu or Asp
  misc_feature (82)..(82): Xaa can be any amino acid; or in specific enbodiment, it can be amino acid with an acidic or amide derivative side chain; or in another specific embodiment, Xaa is the amino acid Gln or Glu
  misc_feature (91)..(91): Xaa can be any amino acid; or in specific enbodiment, it can be amino acid with an aliphatic hydroxyl side chain; or in another specific embodiment, Xaa is the amino acid Ser or Thr
QVQLVQSGAEXKKPGASVKXSCKASGYTFTDYYINWVXQAPGKGLEWIARIYPGSGNTYYNEKFKGRXTXTAXKSTSTAYMXLSSLRSEDXAVYFCARGVYYFDYWGQGTTVTVSS

SEQ ID NO: 19 - protein, 464 aa, Artificial Sequence
  heavy chain sequence with mutations
MEWSWVFLFFLSVTTGVHSQVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 234 aa, Artificial Sequence
  light chain sequence
MSVPTQVLGLLLLWLTDARCDIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 445 aa, Artificial Sequence
  mAb1 full-length heavy chain sequence (including Fc)
QVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, Artificial Sequence
  mAb1 full-length light chain sequence
DIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1392 bp, Artificial Sequence
  DNA heavy chain sequence of an anti-KIT antibody
atggagtggtcctgggtgttcctgttctttctgtccgtgaccacaggcgtgcacagccaggtgcagctggtgcagtctggagctgaggtgaagaagccaggagcttctgtgaagctgtcctgcaaggccagcggctacaccttcacagactactatatcaactgggtgagacaggctcctggcaagggcctggagtggatcgctcgcatctatccaggctctggcaacacctactataatgagaagtttaagggccgggccaccctgacagctgataagagcacctctacagcttacatgcagctgtccagcctgagatccgaggacaccgccgtgtacttctgcgctcgcggcgtgtactattttgattattggggccagggcaccacagtgaccgtgtcttccgctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgtacatcacaagggagccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggc

SEQ ID NO: 24 - DNA, 702 bp, Artificial Sequence
  DNA light chain sequence of an anti-KIT antibody
atgtccgtgccaacccaggtgctgggactgctgctgctgtggctgaccgacgccaggtgcgatatcgtgatgacacagtccccttccagcctgtctgcttccgtgggcgacagagtgaccatcacctgtaaggccagccagaacgtgcgcaccaatgtggcttggtaccagcagaagccaggcaaggcccccaaggctctgatctatagcgcctcttacaggtatagcggagtgcctgaccggttcaccggatccggaagcggaacagacttcaccctgacaatctcttccctgcagcctgaggacttcgctgattacttttgccagcagtacaactcttatccaaggaccttcggcggcggcacaaaggtggagatcaagcggaccgtggccgctccaagcgtgttcatctttcccccttctgacgagcagctgaagtctggcacagcctccgtggtgtgcctgctgaacaacttctaccccagagaggccaaggtgcagtggaaggtggataacgctctgcagtctggcaattcccaggagagcgtgaccgagcaggactctaaggattccacatatagcctgagctctaccctgacactgtctaaggccgattacgagaagcacaaggtgtatgcttgcgaggtgacccatcagggcctgtccagcccagtgacaaagtccttcaatcgcggcgagtgt

SEQ ID NO: 25 - protein, 7 aa, Artificial Sequence
  heavy chain CDR1 of anti-KIT antibody (VH-CDR1)
GYTFTDY

SEQ ID NO: 26 - protein, 6 aa, Artificial Sequence
  heavy chain CDR2 of anti-KIT antibody (VH-CDR2)
YPGSGN

SEQ ID NO: 27 - protein, 8 aa, Artificial Sequence
  heavy chain CDR3 of anti-KIT antibody (VH-CDR3 - AbM)
GVYYFDYW

SEQ ID NO: 28 - protein, 7 aa, Artificial Sequence
  light chain CDR1 of anti-KIT antibody (VL-CDR1)
SQNVRTN

SEQ ID NO: 29 - protein, 3 aa, Artificial Sequence
  light chain CDR2 of anti-KIT antibody (VL-CDR2)
SAS

SEQ ID NO: 30 - protein, 6 aa, Artificial Sequence
  light chain CDR3 of anti-KIT antibody (VL-CDR3)
YNSYPR

SEQ ID NO: 31 - protein, 4 aa, Artificial Sequence
  heavy chain CDR2 of anti-KIT antibody (VH-CDR2)
PGSG

SEQ ID NO: 32 - protein, 6 aa, Artificial Sequence
  heavy chain CDR3 of anti-KIT antibody (VH-CDR3)
VYYFDY

SEQ ID NO: 33 - protein, 10 aa, Artificial Sequence
  heavy chain CDR1 of anti-KIT antibody (VH-CDR1 - AbM)
GYTFTDYYIN

SEQ ID NO: 34 - protein, 10 aa, Artificial Sequence
  heavy chain CDR2 of anti-KIT antibody (VH-CDR2 - AbM)
RIYPGSGNTY

SEQ ID NO: 35 - protein, 7 aa, Artificial Sequence
  light chain CDR1 of anti-KIT antibody (VL-CDR1 - Contact)
RTNVAWY

SEQ ID NO: 36 - protein, 10 aa, Artificial Sequence
  light chain CDR2 of anti-KIT antibody (VL-CDR2 - Contact)
ALIYSASYRY

SEQ ID NO: 37 - protein, 8 aa, Artificial Sequence
  light chain CDR3 of anti-KIT antibody (VL-CDR3 - Contact)
QQYNSYPR

SEQ ID NO: 38 - protein, 6 aa, Artificial Sequence
  heavy chain CDR1 of anti-KIT antibody (VH-CDR1 - Contact)
TDYYIN

SEQ ID NO: 39 - protein, 13 aa, Artificial Sequence
  heavy chain CDR2 of anti-KIT antibody (VH-CDR2 - Contact)
WIARIYPGSGNTY

SEQ ID NO: 40 - protein, 9 aa, Artificial Sequence
  heavy chain CDR3 of anti-KIT antibody (VH-CDR3 - Contact)
ARGVYYFDY